(19) United States
(12) Patent Application Publication
(10) Pub. No.: US 2025/0263363 A1
(43) Pub. Date: Aug. 21, 2025

(54) OLIGOMERIC BINAPHTYL COMPOUNDS AND THERMOPLASTIC RESINS

(71) Applicant: MITSUBISHI GAS CHEMICAL COMPANY, INC., Tokyo (JP)

(72) Inventors: Noriyuki KATO, Tokyo (JP); Katsushi NISHIMORI, Tokyo (JP); Atsushi MOTEGI, Tokyo (JP); Kentaro ISHIHARA, Tokyo (JP); Takafumi WATANABE, Tokyo (JP); Kazutaka TAKAMATSU, Tokyo (JP); Yutaro HARADA, Tokyo (JP); Karl REUTER, Freiburg (DE); Philipp KOSCHKER, Freiburg (DE); Vasyl ANDRUSHKO, Freiburg (DE); Andreas KYRI, Freiburg (DE); Florian STOLZ, Freiburg (DE); Mark KANTOR, Freiburg (DE)

(73) Assignee: MITSUBISHI GAS CHEMICAL COMPANY, INC., Tokyo (JP)

(21) Appl. No.: 18/858,517

(22) PCT Filed: Apr. 24, 2023
(86) PCT No.: PCT/JP2023/016993

§ 371 Date: Oct. 21, 2024

(30) Foreign Application Priority Data
Apr. 26, 2022 (JP) 2022-072635

(51) Int. Cl. C07C 43/23; C07C 59/64; C08G 65/34

(52) U.S. Cl. CPC C07C 43/23; C07C 59/64; C08G 65/34; C07C 2602/26

(57) ABSTRACT

Oligomeric binaphtyl compounds of formula (I),

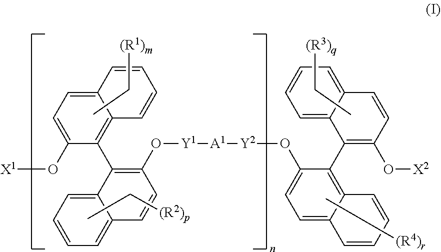

where
X¹-X² are selected from hydrogen, -Alk¹-OH, —CH₂-A²-CH₂—OH, -Alk²-C(O)OR^{x}, —CH₂-A²-C(O)OR^{x} and —C(O)-A²-C(O)OR^{x}, where R^{x} is selected from the group consisting of hydrogen, phenyl, benzyl and C₁-C₄-alkyl;
Y¹-Y² are selected from —CH₂—, —CHAr^{Y}- and —CH(CH₂Ar^{Y})—;
A¹ is a single bond, —CH₂—, —CHAr^{A}—, —CH(CH₂Ar^{A})-, —C(CH₂Ar^{A})₂-, a moiety of the formula(A), mono- or polycyclic arylene having 6-26 carbon atoms as ring members or mono- or polycyclic hetarylene having 5-26 atoms, which are ring members, alternatively, the moiety —Y¹-A¹-Y²— in formula(I) is —CH₂— or —CHAr^{Y}-,
n is 1, 2 or 3; m, p, q and r are 0, 1 or 2;
R¹-R⁴ are independently-selected from the group consisting of halogen, C₂-C₃-alkynyl, CN, R, OR, CHₛR′₃₋ₛ, NR₂, C(O)R and CH═CHR″, R¹, R², R³ or R⁴ is identical or different if more than 1 of R¹, R², R³ or R⁴ is present, where s on each occurrence is 0, 1 or 2.

The present invention relates to oligomeric binaphtyl compounds that are suitable as monomers for preparing thermoplastic resins, such as polycarbonate resins, which have beneficial optical and mechanical properties and can be used for producing optical devices.

### BACKGROUND OF INVENTION

Optical devices, such as optical lenses made of optical resin instead of optical glass are advantageous in that they can be produced in large numbers by injection molding. Nowadays, optical resins, in particular, transparent polycarbonate resins, are frequently used for producing camera lenses. In this regard, resins with a higher refractive index are highly desirable, as they allow for reducing the size and weight of final products. In general, when using an optical material with a higher refractive index, a lens element of the same refractive power can be achieved with a surface having less curvature, so that the amount of aberration generated on this surface can be reduced. As a result, it is possible to reduce the number of lenses, to reduce the eccentric sensitivity of lenses and/or to reduce the lens thickness to thereby achieve weight reduction.

U.S. Pat. No. 9,360,593 describes polycarbonate resins having repeating units derived from binaphthyl monomers of the formula (A):

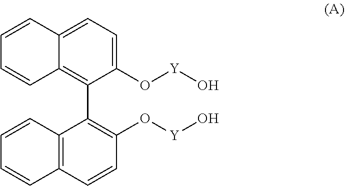

where Y is C₁-C₄-alkandiyl, in particular 1,2-ethandiyl. It is said that the polycarbonate resins have beneficial optical properties in terms of a high refractive index, a low Abbe's number, a high degree of transparency, low birefringence, and a glass transition temperature suitable for injection molding.

Co-Polycarbonates of monomers of the formula (A) with 10,10-bis(4-hydroxyphenyl)anthrone monomers and their use for preparing optical lenses are described in US 2016/0319069.

WO 2019/043060 describes thermoplastic resins for producing optical materials, where the thermoplastic resins comprise a polymerized compound of formula (B)

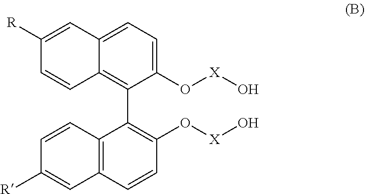

where
X is e.g. C₂-C₄-alkandiyl;
R and R′ are identical or different and selected from optionally substituted mono or polycyclic aryl having from 6 to 36 carbon atoms and optionally substituted mono- or polycyclic hetaryl having a total of 5 to 36 atoms.

However, as observed by the inventors of the present application, binaphtyl derived monomers, such as those of formulae A and B above, despite their multiple advantages, suffer from the disadvantage that they form a significant proportion of undesirable cyclic oligomers when used as monomers in the production of thermoplastic resins such as in the production of polyesters and polycarbonates. These cyclic oligomes may aggravate the molecular weight build-up and/or worsen the product properties of the resin, such as reduced mechanical strength, lower glass transition temperature and/or optical properties. Unfortunately those cyclic components can hardly be removed from the resin in an efficient way. To reduce the formation of such cyclic compounds, it is typically necessary to polymerize the binpathtyl-containing monomers with relatively high amounts of co-monomers.

Without being bound to theory it is assumed that the reason for the increased formation of cyclic compounds when using these monomers is related to their flexible linker units (see moieties —Y—OH and —X—OH in formulae A and B) in combination with the close spatial proximity of the 1- and 1′-positions of the naphthyl residue to which these linkers are connected.

The inventors now found that these problems can be alleviated by the compounds of the formula (I) as described below. The use of the compounds of the formula (I) as monomers in the production of thermoplastic resins, in particular polycarbonates, will yield resins having a reduced content of undesirable cyclic oligomers and/or higher molecular weight and higher refractive index and thus have improved optical properties and/or improved mechanical properties.

Therefore, a first aspect of the present invention relates to the use of the compound of the formula (I) or a mixture thereof

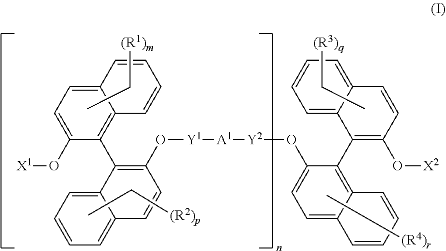

where
X¹ and X² are independently selected from hydrogen, -Alk¹-OH, —CH₂-A²-CH₂—OH, -Alk²-C(O)OR^{x}, —CH₂-A²-C(O)OR^{x} and —C(O)-A²-C(O)OR^{x}, where R^{x} is selected from the group consisting of hydrogen, phenyl, benzyl and C₁-C₄-alkyl;
Y¹ and Y² are independently selected from —CH₂—, —CHAr^{Y}- and —CH(CH₂Ar^{Y})-;
A¹ is selected from the group consisting of a single bond, —CH₂—, —CHAr^{A}—, —CH(CH₂Ar^{A})-, —C(CH₂Ar^{A})₂-, a moiety of the formula (A), mono- or polycyclic arylene having from 6 to 26 carbon atoms as ring members and mono- or polycyclic hetarylene having a total of 5 to 26 atoms, which are ring members, where 1, 2, 3 or 4 of these ring member atoms of hetarylene are selected from nitrogen, sulfur and oxygen, while the remainder of these ring member atoms of hetarylene are carbon atoms, where mono- or polycyclic arylene and mono- or polycyclic hetarylene are unsubstituted or carry 1, 2, 3 or 4 radicals R^{Ar},

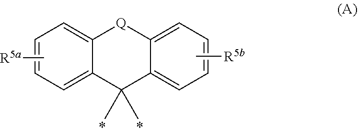

where
Q represents a single bond, O, C═O, CH₂, S or SO₂;
R^{5a}, R^{5b}, independently of each other are selected from the group consisting of hydrogen, fluorine, CN, R, OR, CHₖR′₃₋ₖ, NR₂, C(O)R and C(O)NH₂, where k is 0, 1 or 2; and
* represents the connection point to Y¹ or Y²;
alternatively, the moiety —Y¹-A¹-Y²— in formula (I) may be —CH₂— or —CHAr^{Y}-,
n is 1, 2 or 3;
R¹, R², R³ and R⁴ are independently selected from the group consisting of halogen, C₂-C₃-alkynyl, CN, R, OR, CHₛR′₃₋ₛ, NR₂, C(O)R and CH═CHR″, it being possible that R¹, R², R³ or R⁴ is identical or different if more than 1 of R¹, R², R³ or R⁴ is present, where s on each occurrence is 0, 1 or 2;
m, p, q and r are independently 0, 1 or 2;
A² is selected from the group consisting of phenylene, naphthylene and biphenylylene;
Alk¹ is C₂-C₄-alkandiyl;
Alk² is C₁-C₄-alkandiyl;
Ar^{Y} and Ar^{A} are selected from the group consisting of mono- or polycyclic aryl having from 6 to 26 carbon atoms as ring atoms and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms, where Ar^{Y} and Ar^{A} are unsubstituted or substituted by 1, 2 or 3 radicals R^{Ar};
R^{Ar} is selected from the group consisting of R, OR, CHₜR′₃₋ₜ, NR₂ and CH═CHR″, where R^{Ar} may identical or different if more than one is present on the same (het)aryl or (het)arylene group, where t on each occurrence is 0, 1 or 2;
R is selected from the group consisting of C₁-C₄-alkyl, phenyl, naphthyl, phenanthrenyl and triphenylenyl, where phenyl, naphthyl, phenanthrenyl and triphenylenyl are unsubstituted or substituted by 1, 2, 3 or 4 identical or different radicals R′″;
R′ is selected from the group consisting of phenyl, naphthyl, phenanthrenyl and triphenylenyl, where phenyl, naphthyl, phenanthrenyl and triphenylenyl are unsubstituted or substituted by 1, 2, 3 or 4 identical or different radicals R′″;
R″ is selected from hydrogen, methyl, phenyl and naphthyl, where phenyl and naphthyl are unsubstituted or substituted by 1, 2, 3 or 4 identical or different radicals
R′″ is selected from the group consisting of phenyl, OCH₃, CH₃, N(CH₃)₂ and C(O)CH₃;
as a monomer for producing a thermoplastic resin, in particular for producing polyesters and especially for producing polycarbonates.

The compounds of the formula (I) are novel, except for those compounds of formula (I), where X¹ and X² are both hydrogen or —CH₂CH₂—OH, Y¹ and Y² are both CH₂, and A¹ is a single bond or CH₂; and except for those compounds of formula (I), where n is 1, X¹ and X² are both hydrogen, Y¹ and Y² are both CH₂, m, p, q and r are all 0, and A¹ is 1,2-phenylene, 1,3-phenylene, 1,6-pyrenylene, 4,4′-biphenylylene, 2,6-pyridinylene, 4,4″-meta-terphenylylene, 2,5-[1,3,4]-thiadiazolylene, 2,5-[1,3,4]-oxadiazolylene, 2,5-thienediyl-bis(4,1-phenylenemethylene), 9,9-diethyl-2,7-9H-fluorenylene, 10-methyl-3,7-phenothiazinylene or 10-ethyl-3,7-phenothiazinylene. These compounds are known from A. R. Abreu et al., Terahedron 2010, 66(3), 743-749; S. C. Jha et al., Synthetic Communications 2003, 33(6), 1005-1009; F. De Jong et al., Journal of the Chemical Society, Chemical Communications 1975, 14, 551-553; E. P. Kyba et al., Journal of Organic Chemistry 1977, 42(26), 4173-4184; DE 2414188 A1; A. R. Abreu et al., Journal of Molecular Catalysis A; Chemical 2010, 325(1-2), 91-97; G. Gao et al., European Journal of Organic Chemistry 2011, 2011(26), 5039-5046; DE 2539324 A1; JP 2015129266 A; H. Egami et al., Journal of the American Chemical Society 2018, 140(8), 2785-2788; F. Peixoto et al., Current Organic Synthesis 2014, 11(2), 301-309; A. R. Abreu et al., Chemistry Letters 2013, 42(1), 37-39; G. W. Gokel et al., Journal of the Chemical Society, Chemical Communications 1975, (11), 444-446; K. Takaishi et al., Journal of the American Chemical Society 2020, 142(4), 1774-1779; H. K. Matsui et al., Bulletin of the Chemical Society of Japan 2000, 73(4), 991-997; K. Ogura, Tetrahedron Letters 1999, 40(51), 9065-9068; P. Rajakumar et al., Supramolecular Chemistry 2009, 21(8), 674-680; R. Kanagalatha et al., Asian Journal of Chemistry 2015, 27(12), 4373-4378; R. Sebastian et al., Journal of Heterocyclic Chemistry 2016, 53(3), 993-996; A. Thirunarayanan et al., RSC Advances 2014, 4(45), 23433-23439; and P. Rajakumar et al., Tetrahedron 2001, 57(48), 9749-9754.

Therefore, a second aspect relates to compounds of the formula (I) that are novel. In other words, the second aspect relates to compounds of the formula (I) except for those compounds of formula (I), where the combination of X¹, X², Y¹, Y² and Ar¹ is as follows:
X¹ and X² are both hydrogen or —CH₂CH₂—OH, Y¹ and Y² are both CH₂, and A¹ is a single bond or CH₂;
and also except for those compounds of formula (I), where the combination of n, m, p, q, r, X¹, X², Y¹, Y² and Ar¹ is as follows:
n is 1, X¹ and X² are both hydrogen, Y¹ and Y² are both CH₂, m, p, q and r are all 0, and A¹ is 1,2-phenylene, 1,3-phenylene, 1,6-pyrenylene, 4,4′-biphenylylene, 2,6-pyridinylene, 4,4″-meta-terphenylylene, 2,5-[1,3,4]-thiadiazolylene, 2,5-[1,3,4]-oxadiazolylene, 2,5-thienediyl-bis(4,1-phenylenemethylene), 9,9-diethyl-2,7-9H-fluorenylene, 10-methyl-3,7-phenothiazinylene or 10-ethyl-3,7-phenothiazinylene.

A third aspect relates to a thermoplastic resin comprising a polymerized unit of the compound of formula (I), i.e. a thermoplastic resin comprising a structural unit represented by formula (III) below;

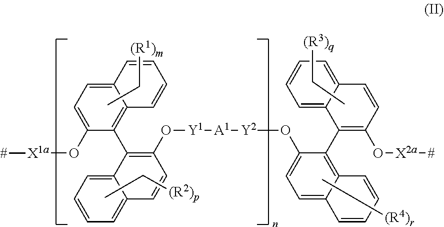

where
#represents a connection point to a neighboring structural unit;
and where X^{1a} and X^{2a}, respectively, is derived from X¹ or X² in formula (I), if X¹ or X² is hydrogen, by replacing hydrogen with a single bond, or, if X¹ or X² is not hydrogen, by replacing the —OH or —OR^{x} group of X¹ or X² with an oxo (—O—) unit, and where X¹, X², Y¹, Y², A¹, R¹, R², R³, R⁴, n, m, p, q and r are as defined herein above.

The invention further relates to an optical device made of a thermoplastic resin as defined above, in particular from a polyester and especially from a polycarbonate.

### DETAILED DESCRIPTION OF INVENTION

The compounds of formula (I) may have axial chirality due to the limited rotation along the bonds between the naphthalene units and therefore compounds of the formula (I) may exist in the form of one of the at least 2⁽ⁿ⁺¹⁾ stereoisomers or in the form of any mixture of these stereoisomers, where n is the variable given in formula (I). Specific examples of these mixtures are those of the respective two stereoisomers belonging to one of the at least 2ⁿ pairs of enantiomers. The present invention relates to the pure stereoisomers of the compounds of formula (I) and the mixtures of any of the stereoisomers, including racemic as well as non-racemic mixtures of the respective two stereoisomers which together constitute an enantiomeric pair.

In terms of the present invention, the term “C₁-C₄-alkandiyl group” may alternatively also be designated “alkylene group having 1, 2, 3 or 4 carbon atoms” and refers to a bivalent, saturated, aliphatic hydrocarbon radical having 1, 2, 3 or 4 carbon atoms. Examples of C₁-C₄-alkandiyl are in particular the methylene group (CH₂), linear alkandiyl such as 1,2-ethandiyl (CH₂CH₂), 1,3-propandiyl (CH₂CH₂CH₂) and 1,4-butdandiyl (CH₂CH₂CH₂CH₂), but also branched alkandiyl such as 1-methyl-1,2-ethandiyl, 1-methyl-1,2-propandiyl, 2-methyl-1,2-propandiyl, 2-methyl-1,3-propandiyl and 1,3-butandiyl.

In terms of the present invention, the term “monocyclic aryl” refers to a monovalent aromatic monocyclic radical, such as in particular phenyl.

In terms of the present invention, the term “monocyclic hetaryl” refers to a monovalent heteroaromatic monocyclic radical, i.e. a heteroaromatic monocycle linked by a single covalent bond to the remainder of the molecule, where the ring member atoms are part of a conjugate π-electron system, where the heteroaromatic monocycle has 5 or 6 ring atoms, which comprise as heterocyclic ring members 1, 2, 3 or 4 nitrogen atoms or 1 oxygen atom and 0, 1, 2 or 3 nitrogen atoms, or 1 sulphur atom and 0, 1, 2 or 3 nitrogen atoms, where the remaining ring atoms are carbon atoms. Examples include furyl (=furanyl), pyrrolyl (=1H-pyrrolyl), thienyl (=thiophenyl), imidazolyl (=1H-imidazolyl), pyrazolyl (=1H-pyrazolyl), 1,2,3-triazolyl, 1,2,4-triazolyl, tetrazolyl, oxazolyl, thiazolyl, isoxazolyl, isothiazolyl, 1,3,4-oxadiazolyl, 1,3,4-thiadiazolyl, pyridyl (=pyridinyl), pyrazinyl, pyridazinyl, pyrimidinyl and triazinyl.

In terms of the present invention, the term “mono- or polycyclic aryl” refers to a monovalent aromatic monocyclic radical as defined herein or to a monovalent aromatic polycyclic radical, i.e. a polycyclic arene linked by a single covalent bond to the remainder of the molecule, where the polycyclic arene is
(i) an aromatic polycyclic hydrocarbon, i.e. a completely unsaturated polycyclic hydrocarbon, where each of the carbon atoms is part of a conjugate π-electron system,
(ii) a polycyclic hydrocarbon which bears at least 1 phenyl ring which is fused to a saturated or unsaturated 4 to 10-membered mono- or bicyclic hydrocarbon ring,
(iii) a polycyclic hydrocarbon which bears at least 2 phenyl rings which are linked to each other by a covalent bond or which are fused to each other directly and/or which are fused to a saturated or unsaturated 4 to 10-membered mono- or bicyclic hydrocarbon ring.

Mono- or polycyclic aryl has from 6 to 26, often from 6 to 24 carbon atoms, e.g. 6, 9, 10, 12, 13, 14, 16, 17, 18, 19, 20, 22 or 24 carbon atoms as ring atoms, in particular from 6 to 20 carbon atoms, especially 6, 10, 12, 13, 14, 16, 17 or 18 carbon atoms. Polycyclic aryl typically has 10 to 26 carbon atoms as ring atoms, in particular from 10 to 20 carbon atoms, especially 10, 12, 13, 14, 16, 17 or 18 carbon atoms.

In this context, polycyclic aryl bearing 2, 3 or 4 phenyl rings which are linked to each other via a single bond include e.g. biphenylyl and terphenylyl. Polycyclic aryl bearing 2, 3 or 4 phenyl rings which are directly fused to each other include e.g. naphthyl, anthracenyl, phenanthrenyl, pyrenyl, triphenylenyl, chrysenyl and benzo[c]phenanthrenyl. Polycyclic aryl bearing 2, 3 or 4 phenyl rings which are fused to a saturated or unsaturated 4- to 10-membered mono- or bicyclic hydrocarbon ring include e.g. 9H-fluorenyl, biphenylenyl, tetraphenylenyl, acenaphthenyl (1,2-dihydroacenaphthylenyl), acenaphthylenyl, 9,10-dihydroanthracen-1-yl, 1,2,3,4-tetrahydrophenanthrenyl, 5,6,7,8-tetrahydrophenanthrenyl, cyclopent[fg]acenaphthylenyl, phenalenyl, fluoranthenyl, benzo[k]fluoranthenyl, perylenyl, 9,10-dihydro-9,10[1′,2′]-benzenoanthracenyl, dibenzo[a,e][8]annulenyl, 9,9′-spirobi[9H-fluoren]yl and spiro[1H-cyclobuta[de]naphthalene-1,9′-[9H]fluoren]yl.

Mono- or polycylic aryl includes, by way of example phenyl, naphthyl, 9H-fluorenyl, phenanthryl, anthracenyl, pyrenyl, chrysenyl, benzo[c]phenanthrenyl, acenaphthenyl, acenaphthylenyl, 2,3-dihydro-1H-indenyl, 5,6,7,8-tetrahydro-naphthalenyl, cyclopent[fg]acenaphthylenyl, 2,3-dihydrophenalenyl, 9,10-dihydroanthracen-1-yl, 1,2,3,4-tetrahydrophenanthrenyl, 5,6,7,8-tetrahydrophenanthrenyl, fluoranthenyl, benzo[k]fluoranthenyl, biphenylenyl, triphenylenyl, tetraphenylenyl, 1,2-dihydroacenaphthylenyl, dibenzo[a,e][8]annulenyl, perylenyl, biphenylyl, terphenylyl, naphthylenphenyl, phenanthrylphenyl, anthracenylphenyl, pyrenylphenyl, 9H-fluorenylphenyl, di(naphthylen)phenyl, naphthylenbiphenyl, tri(phenyl)phenyl, tetra(phenyl)phenyl, pentaphenyl(phenyl), phenylnaphthyl, binaphthyl, phenanthrylnaphthyl, pyrenylnaphthyl, phenylanthracenyl, biphenylanthracenyl, naphthalenylanthracenyl, phenanthrylanthracenyl, dibenzo[a,e][8]annulenyl, 9,10-dihydro-9,10[1′,2′]benzoanthracenyl, 9,9′-spirobi-9H-fluorenyl and spiro[1H-cyclobuta[de]naphthalene-1,9′-[9H]fluoren]yl.

In terms of the present invention, the term “mono- or polycyclic hetaryl” refers to a monovalent heteroaromatic monocyclic radical as defined herein or to a monovalent heteroaromatic polycyclic radical, i.e. a polycyclic hetarene linked by a single covalent bond to the remainder of the molecule, where
(i) the polycyclic hetarene bears a heteroaromatic monocycle as defined above and at least one, e.g. 1, 2, 3, 4 or 5, further aromatic rings selected from phenyl and heteroaromatic monocycles as defined above, where the aromatic rings of the polycyclic hetarene are linked to each other by a covalent bond and/or fused to each other directly and/or fused to a saturated or unsaturated 4 to 10-membered mono- or bicyclic hydrocarbon ring, or
(ii) the polycyclic hetarene bears at least one saturated or partially or fully unsaturated 5-, 6-, 7- or 8-membered heterocyclic ring bearing 1, 2 or 3 heteroatoms selected from oxygen, sulphur and nitrogen as ring atoms, such as 2H-pyran, 4H-pyran, thiopyran, 1,4-dihydropyridin, 4H-1,4-oxazin, 4H-1,4-thiazin, 1,4-dioxin, oxepin, thiepin, dioxin, dithiin, dioxepin, dithiepin, dioxocine, dithiocine and at least one, e.g. 1, 2, 3, 4 or 5, aromatic rings selected from phenyl and heteroaromatic monocycles as defined above, where at least one of the aromatic rings is directly fused to the saturated or partially unsaturated 5- to 8-membered heterocyclic ring and where the aromatic rings of the polycyclic hetarene are linked to each other by a covalent bond or fused to each other directly and/or fused to a saturated or unsaturated 4 to 10-membered mono- or bicyclic hydrocarbon ring.

Mono- or polycyclic hetaryl has from 5 to 26, often from 5 to 24 ring atoms, in particular 5 to 20 ring atoms, which comprise 1, 2, 3 or 4 atoms selected from nitrogen atoms, sulphur atoms and oxygen atoms, where the remainder of the ring atoms are carbon atoms. Polycyclic hetaryl generally has from 9 to 26, often from 9 to 24 ring atoms, in particular 9 to 20 ring atoms, which comprise 1, 2, 3 or 4 atoms selected from nitrogen atoms, sulphur atoms and oxygen atoms, where the remainder of the ring atoms are carbon atoms.

Examples of polycyclic hetaryl include, but are not limited to, benzofuryl, benzothienyl, dibenzofuranyl (=dibenzo[b,d]furanyl), dibenzothienyl (=dibenzo[b,d]thienyl), naphthofuryl, naphthothienyl, furo[3,2-b]furanyl, furo[2,3-b]furanyl, furo[3,4-b]furanyl, thieno[3,2-b]thienyl, thieno[2,3-b]thienyl, thieno[3,4-b]thienyl, oxanthrenyl, thianthrenyl, indolyl (=1H-indolyl), isoindolyl (=2H-isoindolyl), carbazolyl, indolizinyl, benzopyrazolyl, benzimidazolyl, benzoxazolyl, benzothiazolyl, benzo[c,d]indolyl, 1H-benzo[g]indolyl, quinolinyl, isoquinolinyl, acridinyl, phenazinyl, quinazolinyl, quinoxalinyl, phenoxazinyl, phenthiazinyl, benzo[b][1,5]naphthyridinyl, cinnolinyl, 1,5-naphthyridinyl, 1,8-naphthyridinyl, phenylpyrrolyl, naphthylpyrrolyl, dipyridyl, phenylpyridyl, naphthylpyridyl, pyrido[4,3-b]indolyl, pyrido[3,2-b]indolyl, pyrido[3,2-g]quinolinyl, pyrido[2,3-b][1,8]naphthyridinyl, pyrrolo[3,2-b]pyridinyl, pteridinyl, puryl, 9H-xanthenyl, 9H-thioxanthenyl, 2H-chromenyl, 2H-thiochromenyl, phenanthridinyl, phenanthrolinyl, benzo[1,2-b:4,3-b′]difuranyl, benzo[1,2-b:6,5-b′]difuranyl, benzo[1,2-b:5,4-b′]difuranyl, benzo[1,2-b:4,5-b′]difuranyl, naphthofuranyl, benzo[b]naphtho[1,2-d]furanyl, benzo[b]naphtho[2,3-d]furanyl, benzo[b]naphtho[2,1-d]furanyl, tribenzo[b,d,f]oxepinyl, dibenzo[b,d]thienyl, naphtho[1,2-b]thienyl, naphtho[2,3-b]thienyl, naphtho[2,1-b]thienyl, benzo[b]naphtho[1,2-d]thienyl, benzo[b]naphtho[2,3-d]thienyl, benzo[b]naphtho[2,1-d]thienyl, 6H-dibenzo[b,d]thiopyranyl, 5H,9H-[1]benzothiopyrano[5,4,3-c,d,e][2]benzothiopyranyl, 5H,10H-[1]benzothiopyrano[5,4,3-c,d,e][2]benzothiopyranyl, benzo[1,2-6:4,3-b′]bisthienyl, benzo[1,2-b:6,5-b′]bisthienyl, benzo[1,2-b5,4-b′]bisthienyl, benzo[1,2-6:4,5-b′]bisthienyl, 1,4-benzodithiinyl, naphtho[1,2-b][1,4]dithiinyl, naphtho[2,3-b][1,4]dithiinyl, thianthrenyl, benzo[a]thianthrenyl, benzo[b]thianthrenyl, dibenzo[a,c]thianthrenyl, dibenzo[a,h]thianthrenyl, dibenzo[a,i]thianthrenyl, dibenzo[a,j]thianthrenyl, dibenzo[b,i]thianthrenyl, 2H-naphtho[1,8-b,c]thienyl, 5H-phenanthro[4,5-b,c,d]thiopyranyl, 10,11-dihydrodibenzo[b,f]thiepinyl, 6,7-dihydrodibenzo[b,d]thiepinyl, dibenzo[b,f]thiepinyl, dibenzo[b,d]thiepinyl, 6H-dibenzo[d,f][1,3]dithiepinyl, tribenzo[b,d,f]thiepinyl, benzothieno[3,4-c,d]thieno[2,3,4-j,k][2]benzothiepinyl, dinaphtho[1,8-bc:1′,8′-f,g][1,5]dithiocinyl, furo[3,2-g]quinolinyl, furo[2,3-g]quinolinyl, furo[2,3-g]quinoxalinyl, benzo[g]chromenyl, thieno[3,2-f][1]benzothienyl, thieno[2,3-f][1]benzothienyl, thieno[3,2-g]quinolinyl, thieno[2,3-g]quinolinyl, thieno[2,3-g]quinoxalinyl, benzo[g]thiochromenyl, pyrrolo[3,2,1-h,i]indolyl, benzo[g]quinoxalinyl, benzo[f]quinoxalinyl, and benzo[h]isoquinolinyl.

In terms of the present invention, the term “monocyclic arylene” refers to a bivalent aromatic monocyclic radical, such as in particular phenylene.

In terms of the present invention, the term “monocyclic hetarylene” refers to a bivalent heteroaromatic monocyclic radical, i.e. a heteroaromatic monocycle linked by two single covalent bonds to the two remaining parts of the molecule, where the ring member atoms are part of a conjugate n-electron system, where the heteroaromatic monocycle has 5 or 6 ring atoms, which comprise as heterocyclic ring members 1, 2, 3 or 4 nitrogen atoms or 1 oxygen atom and 0, 1, 2 or 3 nitrogen atoms, or 1 sulphur atom and 0, 1, 2 or 3 nitrogen atoms, where the remaining ring atoms are carbon atoms. Examples include furylene (=furanylene), pyrrolylene (=1H-pyrrolylene), thienylene (=thiophenylene), imidazolylene (=1H-imidazolylene), pyrazolylene (=1H-pyrazolylene), 1,2,3-triazolylene, 1,2,4-triazolylene, tetrazolylene, oxazolylene, thiazolylene, isoxazolylene, isothiazolylene, 1,3,4-oxadiazolylene, 1,3,4-thiadiazolylene, pyridylene (=pyridinylene), pyrazinylene, pyridazinylene, pyrimidinylene and triazinylene.

In terms of the present invention, the term “mono- or polycyclic arylene” refers to a bivalent aromatic monocyclic radical as defined herein or to a bivalent aromatic polycyclic radical, i.e. a polycyclic arene linked by two single covalent bonds to the two remaining parts of the molecule, where the polycyclic arene is
(i) an aromatic polycyclic hydrocarbon, i.e. a completely unsaturated polycyclic hydrocarbon, where each of the carbon atoms is part of a conjugate π-electron system,
(ii) a polycyclic hydrocarbon which bears at least 1 phenyl ring which is fused to a saturated or unsaturated 4 to 10-membered mono- or bicyclic hydrocarbon ring,
(iii) a polycyclic hydrocarbon which bears at least 2 phenyl rings which are linked to each other by a covalent bond or which are fused to each other directly and/or which are fused to a saturated or unsaturated 4 to 10-membered mono- or bicyclic hydrocarbon ring.

Mono- or polycyclic arylene has from 6 to 26, often from 6 to 24 carbon atoms, e.g. 6, 9, 10, 12, 13, 14, 16, 17, 18, 19, 20, 22 or 24 carbon atoms as ring atoms, in particular from 6 to 20 carbon atoms, especially 6, 10, 12, 13, 14, 16, 17 or 18 carbon atoms. Polycyclic arylene typically has 10 to 26 carbon atoms as ring atoms, in particular from 10 to 20 carbon atoms, especially 10, 12, 13, 14, 16, 17 or 18 carbon atoms.

In this context, polycyclic arylene bearing 2, 3 or 4 phenyl rings which are linked to each other via a single bond include e.g. biphenylylene and terphenylylene. Polycyclic arylene bearing 2, 3 or 4 phenyl rings which are directly fused to each other include e.g. naphthylene, anthracenylene, phenanthrenylene, pyrenylene, triphenylenylene, chrysenylene and benzo[c]phenanthrenylene. Polycyclic arylene bearing 2, 3 or 4 phenyl rings which are fused to a saturated or unsaturated 4- to 10-membered mono- or bicyclic hydrocarbon ring include e.g. 9H-fluorenylene, biphenylenylene, tetraphenylenylene, acenaphthenylene (1,2-dihydroacenaphthylenylene), acenaphthylenylene, 9,10-dihydroanthracen-1-ylene, 1,2,3,4-tetrahydrophenanthrenylene, 5,6,7,8-tetrahydrophenanthrenylene, cyclopent[fg]acenaphthylenylene, phenalenylene, fluoranthenylene, benzo[k]fluoranthenylene, perylenylene, 9,10-dihydro-9,10[1′,2′]-benzenoanthracenylene, dibenzo[a,e][8]annulenylene, 9,9′-spirobi[9H-fluoren]ylene and spiro[1H-cyclobuta[de]naphthalene-1,9′-[9H]fluoren]ylene.

Mono- or polycylic arylene includes, by way of example phenylene, naphthylene, 9H-fluorenylene, phenanthrylene, anthracenylene, pyrenylene, chrysenylene, benzo[c]phenanthrenylene, acenaphthenylene, acenaphthylenylene, 2,3-dihydro-1H-indenylene, 5,6,7,8-tetrahydro-naphthalenylene, cyclopent[fg]acenaphthylenylene, 2,3-dihydrophenalenylene, 9,10-dihydroanthracen-1-ylene, 1,2,3,4-tetrahydrophenanthrenylene, 5,6,7,8-tetrahydrophenanthrenylene, fluoranthenylene, benzo[k]fluoranthenylene, biphenylenylene, triphenylenylene, tetraphenylenylene, 1,2-dihydroacenaphthylenylene, dibenzo[a,e][8]annulenylene, perylenylene, biphenylylene, terphenylylene, naphthylenphenylene, phenanthrylphenylene, anthracenylphenylene, pyrenylphenylene, 9H-fluorenylphenylene, di(naphthylen)phenylene, naphthylenbiphenylene, tri(phenyl)phenylene, tetra(phenyl)phenylene, pentaphenyl(phenylene), phenyinaphthylene, binaphthylene, phenanthrylnaphthylene, pyrenylnaphthylene, phenylanthracenylene, biphenylanthracenylene, naphthalenylanthracenylene, phenanthrylanthracenylene, dibenzo[a,e][8]annulenylene, 9,10-dihydro-9,10[1′,2′]benzoanthracenylene, 9,9′-spirobi-9H-fluorenylene and spiro[1H-cyclobuta[de]naphthalene-1,9′-[9H]fluoren]ylene.

In terms of the present invention, the term “mono- or polycyclic hetarylene” refers to a bivalent heteroaromatic monocyclic radical as defined herein or to a bivalent heteroaromatic polycyclic radical, i.e. a polycyclic hetarene linked by two single covalent bonds to the two remaining parts of the molecule, where
(i) the polycyclic hetarene bears a heteroaromatic monocycle as defined above and at least one, e.g. 1, 2, 3, 4 or 5, further aromatic rings selected from phenyl and heteroaromatic monocycles as defined above, where the aromatic rings of the polycyclic hetarene are linked to each other by a covalent bond and/or fused to each other directly and/or fused to a saturated or unsaturated 4 to 10-membered mono- or bicyclic hydrocarbon ring, or
(ii) the polycyclic hetarene bears at least one saturated or partially or fully unsaturated 5-, 6-, 7- or 8-membered heterocyclic ring bearing 1, 2 or 3 heteroatoms selected from oxygen, sulphur and nitrogen as ring atoms, such as 2H-pyran, 4H-pyran, thiopyran, 1,4-dihydropyridin, 4H-1,4-oxazin, 4H-1,4-thiazin, 1,4-dioxin, oxepin, thiepin, dioxin, dithiin, dioxepin, dithiepin, dioxocine, dithiocine and at least one, e.g. 1, 2, 3, 4 or 5, aromatic rings selected from phenyl and heteroaromatic monocycles as defined above, where at least one of the aromatic rings is directly fused to the saturated or partially unsaturated 5- to 8-membered heterocyclic ring and where the aromatic rings of the polycyclic hetarene are linked to each other by a covalent bond or fused to each other directly and/or fused to a saturated or unsaturated 4 to 10-membered mono- or bicyclic hydrocarbon ring.

Mono- or polycyclic hetarylene has from 5 to 26, often from 5 to 24 ring atoms, in particular 5 to 20 ring atoms, which comprise 1, 2, 3 or 4 atoms selected from nitrogen atoms, sulphur atoms and oxygen atoms, where the remainder of the ring atoms are carbon atoms. Polycyclic hetaryl generally has from 9 to 26, often from 9 to 24 ring atoms, in particular 9 to 20 ring atoms, which comprise 1, 2, 3 or 4 atoms selected from nitrogen atoms, sulphur atoms and oxygen atoms, where the remainder of the ring atoms are carbon atoms.

Examples of polycyclic hetarylene include, but are not limited to, benzofurylene, benzothienylene, dibenzofuranylene (=dibenzo[b,d]furanylene), dibenzothienylene (=dibenzo[b,d]thienylene), naphthofurylene, naphthothienylene, furo[3,2-b]furanylene, furo[2,3-b]furanylene, furo[3,4-b]furanylene, thieno[3,2-b]thienylene, thieno[2,3-b]thienylene, thieno[3,4-b]thienylene, oxanthrenylene, thianthrenylene, indolylene (=1H-indolylene), isoindolylene (=2H-isoindolylene), carbazolylene, indolizinylene, benzopyrazolylene, benzimidazolylene, benzoxazolylene, benzothiazolylene, benzo[c,d]indolylene, 1H-benzo[g]indolylene, quinolinylene, isoquinolinylene, acridinylene, phenazinylene, quinazolinylene, quinoxalinylene, phenoxazinylene, phenthiazinylene, benzo[b][1,5]naphthyridinylene, cinnolinylene, 1,5-naphthyridinylene, 1,8-naphthyridinylene, phenylpyrrolylene, naphthylpyrrolylene, dipyridylene, phenylpyridylene, naphthylpyridylene, pyrido[4,3-b]indolylene, pyrido[3,2-b]indolylene, pyrido[3,2-g]quinolinylene, pyrido[2,3-b][1,8]naphthyridinylene, pyrrolo[3,2-b]pyridinylene, pteridinylene, purylene, 9H-xanthenylene, 9H-thioxanthenylene, 2H-chromenylene, 2H-thiochromenylene, phenanthridinylene, phenanthrolinylene, benzo[1,2-6:4,3-b′]difuranylene, benzo[1,2-b:6,5-b′]difuranylene, benzo[1,2b:5,4-b′]difuranylene, benzo[1,2-b:4,5-b′]difuranylene, naphthofuranylene, benzo[b]naphtho[1,2-d]furanylene, benzo[b]naphtho[2,3-d]furanylene, benzo[b]naphtho[2,1-d]furanylene, tribenzo[b,d,f]oxepinylene, dibenzo[b,d]thienylene, naphtho[1,2-b]thienylene, naphtho[2,3-b]thienylene, naphtho[2,1-b]thienylene, benzo[b]naphtho[1,2-d]thienylene, benzo[b]naphtho[2,3-d]thienylene, benzo[b]naphtho[2,1-d]thienylene, 6H-dibenzo[b,d]thiopyranylene, 5H,9H-[1]benzothiopyrano[5,4,3-c,d,e][2]benzothiopyranylene, 5H,10H-[1]benzothiopyrano[5,4,3-c,d,e][2]benzothiopyranylene, benzo[1,2-b:4,3-b′]bisthienylene, benzo[1,2-b:6,5-b′]bisthienylene, benzo[1,2-b:5,4-b′]bisthienylene, benzo[1,2-b:4,5-b′]bisthienylene, 1,4-benzodithiinylene, naphtho[1,2-b][1,4]dithiinylene, naphtho[2,3-b][1,4]dithiinylene, thianthrenylene, benzo[a]thianthrenylene, benzo[b]thianthrenylene, dibenzo[a,c]thianthrenylene, dibenzo[a,h]thianthrenylene, dibenzo[a,i]thianthrenylene, dibenzo[a,j]thianthrenylene, dibenzo[b,i]thianthrenylene, 2H-naphtho[1,8-b,c]thienylene, 5H-phenanthro[4,5-b,c,d]thiopyranylene, 10,11-dihydrodibenzo[b,f]thiepinylene, 6,7-dihydrodibenzo[b,d]thiepinylene, dibenzo[b,f]thiepinylene, dibenzo[b,d]thiepinylene, 6H-dibenzo[d,f][1,3]dithiepinylene, tribenzo[b,d,f]thiepinylene, benzothieno[3,4-c,d]thieno[2,3,4-j,k][2]benzothiepinylene, dinaphtho[1,8-bc:1′,8′-f,g][1,5]dithiocinylene, furo[3,2-g]quinolinylene, furo[2,3-g]quinolinylene, furo[2,3-g]quinoxalinylene, benzo[g]chromenylene, thieno[3,2-f][1]benzothienylene, thieno[2,3-f][1]benzothienylene, thieno[3,2-g]quinolinylene, thieno[2,3-g]quinolinylene, thieno[2,3-g]quinoxalinylene, benzo[g]thiochromenylene, pyrrolo[3,2,1-h,i]indolylene, benzo[g]quinoxalinylene, benzo[f]quinoxalinylene, and benzo[h]isoquinolinylene.

In terms of the present invention, the suffix “-ylene” means, as customary in the art, that the respective het(arene) moiety is in the form of its diradikal. Accordingly, the suffix “-ylene”, as e.g. in phenylene or 1,4-phenylene, is used here synonymously with the the suffix “-diyl”, as e.g. in phendiyl or phen-1,4-diyl.

In terms of the present invention, a “structural unit” is a structural element which is present repeatedly in the polymer backbone of the thermoplastic resin. Therefore, the terms “structural unit” and “repeating unit” are used synonymously.

In terms of the present invention, the term “optical device” refers to a device that is transparent for visible light and manipulates light beams, in particular by refraction. Optical devices include but are not limited to prisms, lenses, optical films and combinations thereof, especially lenses for cameras and lenses for glasses.

The remarks made below as to preferred embodiments of the variables (substituents) of the compounds of formula (I) and of the structural units of formula (II) are valid on their own as well as preferably in combination with each other.

The remarks made below concerning preferred embodiments of the variables further are valid on their own as well as preferably in combination with each other concerning the compounds of formula (I) and the structural units of formula (II), where applicable, as well as concerning the uses according to the invention.

In formula (I) and likewise in formula (II), the variables X¹, X², Y¹, Y², R¹, R², R³, R⁴, A¹, n, m, p, q and r on their own or preferably in any combination preferably have the following meanings:

Preference is given to those variables X¹ and X² in formula (I) that are independently selected from hydrogen, -Alk¹-OH, —CH₂-A²-CH₂—OH, -Alk²-C(O)OR^{x} and —CH₂-A²-C(O)OR^{x}, and accordingly to those variables X^{1a} and X^{2a} in formula (II) that are independently selected from -Alk¹-O—, —CH₂-A²-CH₂—O—, -Alk²-C(O)O— and —CH₂-A²-C(O)O—, where Alk¹, -Alk², A² and R^{x} have the meanings defined herein, in particular the preferred meanings.

In a preferred group (1) of embodiments, the variables X¹ and X² in formula (I) are independently selected from -Alk¹-OH and —CH₂-A²-CH₂—OH and accordingly the variables X^{1a} and X^{2a} in formula (II) are independently selected from -Alk¹-O— and —CH₂-A²-CH₂—O—, wherein Alk¹ is preferably a linear C₂-C₄-alkandiyl, such as 1,2-ethandiyl (CH₂—CH₂), 1,3-propandiyl or 1,4-butandiyl, and in particular is 1,2-ethandiyl, and A² is preferably selected from 1,4-phenylene, 1,3-phenylene, 2,6-naphthylene, 1,4-naphthylene, 1,5-naphthylene and 4,4′-biphenylylene. It is also preferred in this context that the variables X¹ and X² in formula (I) or the variables X^{1a} and X²a in formula (II) are identical to each other.

Accordingly, in a particularly preferred subgroup (1.1) of embodiments the variables X¹ and X² in formula (I) are selected from 2-hydroxyethyl (i.e. 2-(HO)-ethyl), hydroxymethyl-phenyl-methyl (i.e. HO-methyl-phenyl-methyl), hydroxymethyl-naphthyl-methyl and hydroxymethyl-biphenylyl-methyl, especially from 2-hydroxyethyl, 4-(hydroxymethyl)phenyl)methyl, (3-(hydroxymethyl)phenyl)methyl, (4-(hydroxymethyl)-1-naphthyl)methyl, (5-(hydroxymethyl)-1-naphthyl)methyl, (6-(hydroxymethyl)-2-naphthyl)methyl and 4′-(hydroxymethyl)-1,1′-biphenylyl-4-methyl, and specifically from 2-hydroxyethyl, 4-(hydroxymethyl)phenyl)methyl and (3-(hydroxymethyl)phenyl)methyl. Correspondingly, in this particularly preferred group (1.1) of embodiments the variables X^{1a} and X^{2a} in formula (II) are selected from 2(-O)-ethyl, —O-methyl-phenyl-methyl and —O-methyl-naphthyl-methyl, especially from 2(-O)-ethyl, (4(-O-methyl)phenyl)methyl, (3(-Omethyl)phenyl)methyl, (4(-O-methyl)-1-naphthyl)methyl, (5(-O-methyl)-1-naphthyl)methyl, (6(-O-methyl)-2-naphthyl)methyl and 4′(-O-methyl)-1,1′-biphenylyl-4-methyl, and specifically from 2(-O)-ethyl, (4(-O-methyl)phenyl)methyl and (3(-O-methyl)phenyl)methyl, (4(-O-methyl)-1-naphthyl)methyl.

In a particular subgroup (1′) of embodiments the variables X¹ and X² in formula (I) have identical meanings and, likewise, the variables X^{1a} and X^{2a} in formula (II) have identical meanings, which are selected from the meanings defined in groups (1) and (1.1), of embodiments.

In another group (2) of embodiments the variables X¹ and X² in formulae (I) and (II) are both hydrogen and accordingly the variables X^{1a} and X^{2a} in formula (II) are both a single bond.

In a preferred group (3) of embodiments, the variables X¹ and X² in formula (I) are independently selected from -Alk²-C(O)OR^{x} and —CH₂-A²-C(O)OR^{x} and accordingly the variables X^{1a} and X^{2a} in formula (11) are independently selected from -Alk²-C(O)O— and —CH₂-A²-C(O)O—, wherein Alk² is preferably a linear C₁-C₄-alkandiyl, such as methylene or 1,2-ethandiyl (CH₂—CH₂), and in particular is methylene, A² is preferably selected from 1,4-phenylene, 1,3-phenylene, 2,6-naphthylene, 1,5-naphthylene and 1,4-naphthylene, and R^{x} is preferably hydrogen or C₁-C₄-alkyl, and in particular is methyl. It is also preferred in this context that the variables X¹ and X² or the variables X^{1a} and X^{2a} are identical to each other.

Accordingly, in a particularly preferred subgroup (3.1) of embodiments the variables X¹ and X² in formula (I) are selected from methoxycarbonyl-methyl (i.e. CH₃O—C(O)-methyl), methoxycarbonyl-phenyl-methyl (i.e. CH₃O—C(O)-phenyl-methyl) and methoxycarbonyl-naphthyl-methyl, especially from methoxycarbonyl-methyl, (4-(methoxycarbonyl)phenyl)methyl, (3-(methoxycarbonyl)phenyl)methyl, (4-(methoxycarbonyl)-1-naphthyl)methyl, (5-(methoxycarbonyl)-1-naphthyl)methyl and (6-(methoxycarbonyl)-2-naphthyl)methyl, and specifically from methoxycarbonyl-methyl, (4-(methoxycarbonyl)phenyl)methyl and (3-(methoxycarbonyl)phenyl)methyl. Correspondingly, in this particularly preferred group (3.1) of embodiments the variables X^{1a} and X^{2a} in formula (II) are selected from —O—C(O)-methyl, —O—C(O)-phenyl-methyl and —O—C(O)-naphthylmethyl, especially from —O—C(O)-methyl, (4(-O—C(O)-phenyl)methyl, (3(-O—C(O)-phenyl)methyl, (4-(-O—C(O)-)-1-naphthyl)methyl, (5-(-O—C(O)-)-1-naphthyl)methyl and (6-(O—C(O)-)-2-naphthyl)methyl, and specifically from —O—C(O)-methyl, (4(-O—C(O)-phenyl)methyl and (3(-O—C(O)-phenyl)methyl.

In a particular subgroup (3′) of embodiments the variables X¹ and X² in formula (I) have identical meanings and, likewise, the variables X^{1a} and X^{2a} in formula (II) have identical meanings, which are selected from the meanings defined in groups (3) and (3.1), of embodiments.

In preferred group (4) of embodiments, which is a combination of groups (1.1), (2) and (3.1) of embodiments, the variables X¹ and X² in formula (I) are selected from hydrogen, 2-hydroxyethyl, methoxycarbonyl-methyl, hydroxymethyl-phenyl-methyl, hydroxymethyl-naphthyl-methyl, hydroxymethyl-biphenylyl-methyl, methoxycarbonyl-phenyl-methyl and methoxycarbonyl-naphthyl-methyl, in particular selected from hydrogen, 2-hydroxyethyl, methoxycarbonyl-methyl, (4-(hydroxymethyl)phenyl)methyl, (3-(hydroxymethyl)phenyl)methyl, (4-(hydroxymethyl)-1-naphthyl)methyl, (5-(hydroxymethyl)-1-naphthyl)methyl, (6-(hydroxymethyl)-2-naphthyl)methyl, 4′-(hydroxymethyl)-1,1′-biphenylyl-4-methyl, (4-(methoxycarbonyl)phenyl)methyl, (3-(methoxycarbonyl)phenyl)methyl, (4-(methoxycarbonyl)-1-naphthyl)methyl, (5-(methoxycarbonyl)-1-naphthyl)methyl and (6-(methoxycarbonyl)-2-naphthyl)methyl, especially selected from hydrogen, 2-hydroxyethyl, methoxycarbonyl-methyl, (4-(hydroxymethyl)phenyl)methyl, (3-(hydroxymethyl)phenyl)methyl, (4-(methoxycarbonyl)phenyl)methyl and (3-(methoxycarbonyl)phenyl)methyl, and specifically selected from hydrogen, 2-hydroxyethyl, (4-(hydroxymethyl)phenyl)methyl and (3-(hydroxymethyl)phenyl)methyl. Correspondingly, in this preferred group (4) of embodiments the variables X^{1a} and X^{2a} in formula (II) are selected from a single bond, 2(-O)-ethyl, —O—C(O)-methyl, —O-methyl-phenyl-methyl, —O— methyl-naphthyl-methyl, —O—C(O)-phenyl-methyl and —O—C(O)-naphthyl-methyl, in particular selected from a single bond, 2(-O)-ethyl, —O—C(O)-methyl, (4(-O-methyl)phenyl)methyl, (3(-O-methyl)phenyl)methyl, (4(-O-methyl)-1-naphthyl)methyl, (5(-O-methyl)-1-naphthyl)methyl, (6(-O-methyl)-2-naphthyl)methyl, (4(-O—C(O)-phenyl)methyl, (3-(-O—C(O)-phenyl)methyl, (4-(-O—C(O)-)-1-naphthyl)methyl, (5-(-O—C(O)-)-1-naphthyl)methyl and (6-(methoxycarbonyl)-2-naphthyl)methyl, especially selected from a single bond, 2(-O)-ethyl, —O—C(O)-methyl, (4(-O-methyl)phenyl)methyl, (3(-O-methyl)phenyl)methyl, 4(-O—C(O)-phenyl)methyl and (3-(-O—C(O)-phenyl)methyl, and specifically selected from a single bond, 2(-O)-ethyl, (4(-O-methyl)phenyl)methyl and (3(O-methyl)phenyl)methyl.

In a particular subgroup (4′) of embodiments the variables X¹ and X² in formula (I) have identical meanings and, likewise, the variables X^{1a} and X^{2a} in formula (II) have identical meanings, which are selected from the meanings defined in group (4) of embodiments.

Preference is given to the variable A¹ in formulae (I) and (II) that is selected from a single bond, —CH₂—, —CHAr^{A}—, —CH(CH₂Ar^{A})-, —C(CH₂Ar^{A})₂-, mono- or polycyclic arylene having from 6 to 26 carbon atoms as ring members and mono- or polycyclic hetarylene having from 5 to 26 atoms as ring members, especially selected from a single bond, —CH₂—, —C(CH₂Ar^{A})₂-, mono- or polycyclic arylene having from 6 to 26 carbon atoms as ring members and mono- or polycyclic hetarylene having from 5 to 26 atoms as ring members, where the mono- or polycyclic arylene and the mono- or polycyclic hetarylene are unsubstituted or carry 1, 2, 3 or 4 radicals R^{Ar}; wherein Ar^{A}, mono- or polycylic arylene, mono- or polycyclic hetarylene and R^{Ar} have the meanings defined herein, in particular those mentioned herein as preferred.

In a preferred group (5) of embodiments the variable A¹ in formulae (I) and (II) is selected from a single bond, —CH₂—, —CHAr^{A}—, —CH(CH₂Ar^{A})- and —C(CH₂Ar^{A})₂-, preferably from single bond, —CH₂—, —CH(CH₂Ar^{A})- and —C(CH₂Ar^{A})₂-, in particular from —CH₂— and —C(CH₂Ar^{A})₂-, and specifically is —C(CH₂Ar^{A})₂-, where Ar^{A} has one of the meanings defined herein, especially those mentioned herein as preferred, and is specifically selected from phenyl, naphth-1-yl, naphth-2-yl, fluoren-2-yl, fluoren-9-yl, phenanthren-9-yl, dibenzo[b,d]thien-2-yl, dibenzo[b,d]thien-3-yl, dibenzo[b,d]thien-4-yl, dibenzo[b,d]furan-2-yl, dibenzo[b,d]furan-3-yl or dibenzo[b,d]furan-4-yl, thianthren-1-yl, thianthren-2-yl, oxanthren-1-yl, oxanthren-2-yl, 9H-xanth-9-yl and 9H-thioxanth-9-yl.

In a preferred subgroup (5.1) of embodiments the variable A¹ in formulae (I) and (II) is —C(CH₂Ar^{A})₂-, where Ar^{A} is selected from phenyl, naphth-1-yl, naphth-2-yl and phenanthren-9-yl

In a preferred group (6) of embodiments the variable A¹ in formula (I) is selected from the group consisting of mono- or polycyclic arylene having from 6 to 26 carbon atoms as ring members and mono- or polycyclic hetarylene having from 5 to 26 atoms as ring members, in particular of mono- or polycyclic aryl having from 6 to 22, especially 6 to 18, carbon atoms as ring members and mono- or polycyclic hetaryl having a total of 5 to 26 atoms as ring members, especially polycyclic hetaryl having a total of 9 to 26 atoms as ring members, where 1, 2, 3 or 4 of these atoms are nitrogen, oxygen or sulfur atoms, and preferably 1, 2 or 3, such as 1 or 2, of these atoms are oxygen or sulfur atoms, while the remainder of these atoms are carbon atoms, where mono- or polycyclic aryl and mono- or polycyclic hetaryl are unsubstituted or carry 1, 2, 3 or 4, especially 1 or 2, radicals R^{Ar}, where R^{Ar} has one of the meanings defined herein, especially one of the meanings mentioned as preferred.

In a more preferred subgroup (6.1) of embodiments, A¹ is selected from phenylene, naphthylene, 1,2-dihydroacenaphthylene, biphenylylene, 9H-fluorenylene, 11H-benzo[a]fluorenylene, 11H-benzo[b]fluorenylene, 7H-benzo[c]fluorenylene, anthracylene, phenanthrylene, benzo[c]phenanthrylene, pyrenylene, chrysenylene, picenylene, triphenylenylene, furanylene, benzo[b]furanylene, dibenzo[b,d]furanylene, naphtho[1,2-b]furanylene, naphtho[2,3-b]furanylene, naphtho[2,1-b]furanylene, benzo[b]naphtho[1,2-d]furanylene, benzo[b]naphtho[2,3-d]furanylene, benzo[b]naphtho[2,1-d]furanylene, benzo[1,2-b:4,3-b′]difuranylene, benzo[1,2-b-6,5-b′]difuranylene, benzo[1,2-b:5,4-b′]difuranylene, benzo[1,2-b:4,5-b′]difuranylene, 9H-xanthylene, tribenzo[b,d,f]oxepinylene, dibenzo[1,4]dioxinylene, 2H-naphtho[1,8-d,e][1,3]dioxinylene, phenoxathiinylene, dinaphtho[2,3-b-2′,3′-d]furanylene, oxanthrenylene, benzo[a]oxanthrenylene, benzo[b]oxanthrenylene, thienylene, benzo[b]thienylene, dibenzo[b,d]thienylene, naphtho[1,2-b]thienylene, naphtho[2,3-b]thienylene, naphtho[2,1-b]thienylene, benzo[b]naphtho[1,2-d]thienylene, benzo[b]naphtho[2,3-d]thienylene, benzo[b]naphtho[2,1-d]thienylene, benzo[1,2-b:4,3-b′]dithienylene, benzo[1,2-b-6,5-b′]dithienylene, benzo[1,2-b-5,4-b′]dithienylene, benzo[1,2-b:4,5-b′]dithienylene, 9H-thioxanthylene, 6H-dibenzo[b,d]thiopyranylene, 1,4-benzodithiinylene, naphtho[1,2-b][1,4]dithiinylene, naphtho[2,3-b][1,4]dithiinylene, thianthrenylene, benzo[a]thianthrenylene, benzo[b]thianthrenylene, dibenzo[a,c]thianthrenylene, dibenzo[a,h]thianthrenylene, dibenzo[a,i]thianthrenylene, dibenzo[a,j]thianthrenylene, dibenzo[b,i]thianthrenylene, 2H-naphtho[1,8-b,c]thienylene, dibenzo[b,d]thiepinylene, dibenzo[b,f]thiepinylene, 5H-phenanthro[4,5-b,c,d]thiopyranylene, tribenzo[b,d,f]thiepinylene, 2,5-dihydronaphtho[1,8-b,c.4,5-b,c′]dithienylene, 2,6-dihydronaphtho[1,8-b,c.5,4-b,c′]dithienylene, tribenzo[a,c,i]thianthrenylene, benzo[b]naphtho[1,8-e,f][1,4]dithiepinylene, dinaphtho[2,3-b:2′,3′-d]thienylene, 5H-phenanthro[1,10-b,c]thienylene, 7H-phenanthro[1,10-c,b]thienylene, dibenzo[d,d′]benzo[1,2-b:4,5-b′]dithienylene and dibenzo[d,d′]benzo[1,2-b:5,4-b′]dithienylene, where the aforementioned mono- or polycyclic aryl and polycyclic hetaryl are unsubstituted or carry 1 or 2 radicals R^{Ar}.

In an especially preferred subgroup (6.2) of embodiments, A¹ is selected from phenylene, naphthylene, thienylene, furanylene, benzo[b]thienylene, benzo[b]furanylene, dibenzo[b,d]thienylene, dibenzo[b,d]furanylene, biphenylylene, 9H-fluorenylene, oxanthrenylene, phenoxathiinylene, thianthrenylene, 9H-xanthylene and 9H-thioxanthylene, where the aforementioned mono- or polycyclic aryl and mono- and polycyclic hetaryl are unsubstituted or carry 1 or 2 radicals R^{Ar}.

In a particularly preferred subgroup (6.3) of embodiments, A¹ is selected from phenylene, naphthylene, dibenzo[b,d]thienylene, biphenylylene, 9H-fluorenylene, oxanthrenylene, phenoxathiinylene, thianthrenylene, 9H-xanthylene and 9H-thioxanthylene, and in particular selected from 1,4-phenylene, 1,2-phenylene, 1,3-phenylene, 2,3-naphthylene, 2,7-naphthylene, 2,6-naphthylene, 1,4-naphthylene, 1,5-naphthylene, 1,8-naphthylene, 4,6-dibenzo[b,d]thienylene, 2,8-dibenzo[b,d]thienylene, 3,7-dibenzo[b,d]thienylene, 3,3′-biphenylylene, 4,4′-biphenylylene, 9,9-9H-fluorenylene, 2,7-9H-fluorenylene, 2,7-oxanthrenylene, 2,8-oxanthrenylene, 1,4-oxanthrenylene, 2,3-oxanthrenylene, 1,6-oxanthrenylene, 1,9-oxanthrenylene, 1,4-phenoxathiinylene, 3,7-phenoxathiinylene, 2,8-phenoxathiinylene, 3,8-phenoxathiinylene, 2,7-thianthrenylene, 2,8-thianthrenylene, 1,4-thianthrenylene, 2,3-thianthrenylene, 1,6-thianthrenylene, 1,9-thianthrenylene, 9,9-9H-xanthylene and 9,9-9H-thioxanthylene, where the aforementioned mono- or polycyclic aryl and polycyclic hetaryl are unsubstituted or carry 1 or 2 radicals R^{Ar}.

A preferred subgroup (6′) of the group (6) of embodiments, relates to compounds of the formula (I), where the moiety A¹ comprises a phenylene ring, which may bear one or two fused rings selected from fused benzene rings and fused 5- or 6-membered heteroaromatic rings. Amongst the compounds of group (6′) of embodiments preference is given to those compounds, wherein the groups Y¹ and Y² are connected in the parapositions of the phenylene ring of A¹. These compounds are also referred to the paraisomers of group (6′) of embodiments. Also preferred are mixtures of the para-isomer with the corresponding meta- or ortho isomer of the compounds of the formula (I) of the group (6′) of embodiments. Amongst the compounds of group (6′) of embodiments, particular preference is given to the compounds of formula (I), where A¹ is 1,4-phenylene and to mixtures thereof with one or both of its isomers, where A¹ is 1,2-phenylene or 1,3-phenylene.

In a specifically preferred subgroup (6.4) of embodiments, A¹ is selected from 1,4-phenylene, 1,2-phenylene, 1,3-phenylene, 2,3-naphthylene, 2,7-naphthylene, 2,6-naphthylene, 1,4-naphthylene, 1,5-naphthylene, 1,8-naphthylene, 4,6-dibenzo[b,d]thienylene, 2,8-dibenzo[b,d]thienylene, 3,3′-biphenylylene, 4,4′-biphenylylene, 9,9-9H-fluorenylene, 2,7-9H-fluorenylene, 2,7-thianthrenylene, 2,8-thianthrenylene, 1,4-thianthrenylene, 2,3-thianthrenylene, 1,6-thianthrenylene, 1,9-thianthrenylene, 9,9-9H-xanthylene and 9,9-9H-thioxanthylene, where the aforementioned mono- or polycyclic aryl and polycyclic hetaryl are unsubstituted or carry 1 or 2 radicals R^{Ar}, and in particular are unsubstituted.

Preference is given to the variables Y¹ and Y² in formulae (I) and (II) that are selected from —CH₂—, —CHAr^{Y}- and —CH(CH₂Ar^{Y})-, where Ar^{Y} has one of the meanings defined herein, especially those mentioned herein as preferred, and is specifically selected from phenyl, naphth-1-yl, naphth-2-yl, fluoren-2-yl, fluoren-9-yl, phenanthren-9-yl, dibenzo[b,d]thien-2-yl, dibenzo[b,d]thien-3-yl, dibenzo[b,d]thien-4-yl, dibenzo[b,a]furan-2-yl, dibenzo[b,d]furan-3-yl or dibenzo[b,d]furan-4-yl, thianthren-1-yl, thianthren-2-yl, oxanthren-1-yl, oxanthren-2-yl, 9H-xanth-9-yl and 9H-thioxanth-9-yl, and more specifically selected from phenyl, naphth-1-yl, naphth-2-yl and phenanthren-9-yl. In this context it is particularly preferred that the variables Y¹ and Y² are identical to each other.

In a particular group (7) of embodiments the variables Y¹ and Y² in formulae (I) and (II) are both —CH₂—.

In a particularly preferred subgroup (7′) of the group (7) of embodiments the variable A¹ has one of the meanings given in group (6) of embodiments, preferably given in group (6.1) of embodiments, more preferably given in group (6.2) of embodiments, in particular given in group (6.3) of embodiments and especially given in group (6.4) of embodiments.

In an alternative group (8) of embodiments the moiety —Y¹-A¹-Y²— in formulae (I) and (II) is —CH₂— or —CHAr^{Y}-, where Ar^{Y} has one of the meanings defined herein, especially those mentioned herein as preferred.

In a preferred subgroup (8.1) of embodiments the moiety —Y¹-A¹-Y²— in formulae (I) and (II) is —CH₂—.

Preferably, the variable n in formulae (I) and (II) is 1 or 2.

In a particular group (9) of embodiments the variable n in formulae (I) and (II) is 1.

If present, the substituents R¹, R², R³ and R⁴ in formulae (I) and (II) have one of the meanings defined herein, especially those mentioned herein as preferred. In this context, it is preferred that R¹, R², R³ and R⁴, if present, all have the same meaning. It is also preferred here that R¹, R², R³ and R⁴, if present, are attached to the corresponding positions of their respective naphthyl units.

The variables m, p, q and r in formulae (I) and (II) are preferably 0, 1 or 2, more preferably 0 or 1, and in particular all have the same meaning.

In a particularly preferred group (10) of embodiments the variables m, p, q and r in formulae (I) and (II) are all 0.

A skilled person will readily appreciate that in the formulae (I) and (II) the meanings of X¹ and X² given in one or more of groups (1), (1.1) and (1′) of embodiments may be combined with the meanings of A¹ according to either one of groups (5) and (5.1) of embodiments or to one of groups (6), (6.1), (6.2), (6.3) and (6.4) of embodiments, with the meanings of Y¹ and Y² according to group (7) of embodiments, with the meaning of n according to group (9) of embodiments and also with the meanings of m, p, q and r according to group (10) of embodiments. A skilled person will also appreciate that in the formulae (I) and (II) the meanings of X¹ and X² given in group (2) of embodiments may be combined with the meanings of A¹ according to either one of groups (5) and (5.1) of embodiments or to one of groups (6), (6.1), (6.2), (6.3) and (6.4) of embodiments, with the meanings of Y¹ and Y² according to group (7) of embodiments, with the meaning of n according to group (9) of embodiments and also with the meanings of m, p, q and r according to group (10) of embodiments. A skilled person will also appreciate that in the formulae (I) and (II) the meanings of X¹ and X² given in one or more of groups (3), (3.1) and (3′) of embodiments may be combined with the meanings of A¹ according to either one of groups (5) and (5.1) of embodiments or to one of groups (6), (6.1), (6.2), (6.3) and (6.4) of embodiments, with the meanings of Y¹ and Y² according to group (7) of embodiments, with the meaning of n according to group (9) of embodiments and also with the meanings of m, p, q and r according to group (10) of embodiments. A skilled person will also appreciate that in the formulae (I) and (II) the meanings of X¹ and X² given in one of groups (4) and (4′) of embodiments may be combined with the meanings of A¹ according to either one of groups (5) and (5.1) of embodiments or to one of groups (6), (6.1), (6.2), (6.3) and (6.4) of embodiments, with the meanings of Y¹ and Y² according to group (7) of embodiments, with the meaning of n according to group (9) of embodiments and also with the meanings of m, p, q and r according to group (10) of embodiments.

Furthermore, a skilled person will also readily appreciate that in the formulae (I) and (II) the meanings of X¹ and X² according to either one or more of groups (1), (1.1) and (1′) of embodiments, or group (2) of embodiments, or one or more of groups (3), (3.1) and (3′) of embodiments, or one of groups (4) and (4′) may be combined with the meanings of —Y¹-A¹-Y²— according to one of groups (8) and (8.1) of embodiments, with the meaning of n according to group (9) of embodiments and also with the meanings of m, p, q and r according to group (10) of embodiments.

Apart from that and if not stated otherwise, the variables Ar^{Y}, Ar^{A}, Q, R¹, R², R³, R⁴, R^{5a}, R^{5b}, R^{Ar}, R, R′, R″ and R′″ either alone or preferably in combination with each other and with the meanings and preferred meanings of the variables X¹, X¹, A¹, A², n, m, p, q, r, R^{x}, Alk¹ and Alk² described above, have the following meanings.

Ar^{Y} and Ar^{A} are preferably, independently of one another, selected from mono- or polycyclic aryl having from 6 to 18 carbon atoms as ring member atoms and polycyclic hetaryl having a total of 9 to 16 atoms, which are ring member atoms, where 1 or 2 of these ring member atoms of hetaryl are sulfur or oxygen atoms, while the remainder of these ring member atoms of hetaryl are carbon atoms, where mono- or polycyclic aryl and polycyclic hetaryl are unsubstituted or carry 1 or 2 radicals R^{Ar}, where R^{Ar} has one of the meanings defined herein, especially one of the meanings mentioned as preferred. Preference is given here to unsubstituted radicals Ar^{Y} and Ar^{A}.

More preferably, Ar^{Y} and Ar^{A} are independently selected from phenyl, naphthyl, such as naphth-1-yl or naphth-2-yl, fluorenyl, such as fluoren-1-yl, fluoren-2-yl, fluoren-3-yl, fluoren-4-yl, fluoren-9-yl, 11H-benzo[a]fluorenyl, such as 11H-benzo[a]fluoren-7-yl, 11H-benzo[b]fluorenyl, such as 11H-benzo[b]fluoren-1-yl, 7H-benzo[c]fluorenyl, such as 7H-benzo[c]fluoren-5-yl or 7H-benzo[c]fluoren-10-yl, phenanthrenyl, such as phenanthren-1-yl, phenanthren-2-yl, phenanthren-3-yl, phenanthren-4-yl or phenanthren-9-yl, biphenylyl, such as biphenyl-4-yl, biphenyl-3-yl or biphenyl-2-yl, benzo[c]phenanthrenyl, such as benzo[c]phenanthren-1-yl, benzo[c]phenanthren-2-yl, benzo[c]phenanthren-3-yl, benzo[c]phenanthren-4-yl, benzo[c]phenanthren-5-yl or benzo[c]phenanthren-6-yl, pyrenyl, such as pyren-1-yl, pyren-2-yl or pyren-4-yl, chrysenyl, such as chrysen-1-yl, chrysen-2-yl, chrysen-3-yl, chrysen-4-yl, chrysen-5-yl or chrysen-6-yl, triphenylenyl, such as triphenylen-1-yl or triphenylen-2-yl, benzo[b]thienyl, such as benzo[b]thien-2-yl, benzo[b]thien-3-yl, benzo[b]thien-4-yl, benzo[b]thien-5-yl, benzo[b]thien-6-yl or benzo[b]thien-7-yl, dibenzo[b,d]thienyl, such as dibenzo[b,a]thien-1-yl, dibenzo[b,d]thien-2-yl, dibenzo[b,d]thien-3-yl or dibenzo[b,d]thien-4-yl, dibenzo[b,a]furanyl, such as dibenzo[b,a]furan-1-yl, dibenzo[b,d]furan-2-yl, dibenzo[b,d]furan-3-yl or dibenzo[b,d]furan-4-yl, naphtho[1,2-b]thienyl, such as naphtho[1,2-b]thien-5-yl, naphtho[2,3-b]thienyl, such as naphtho[2,3-b]thien-3-yl, naphtho[2,3-b]thien-4-yl or naphtho[2,3-b]thien-9-yl, naphtho[2,1-b]thienyl, such as naphtho[2,1-b]thien-2-yl or naphtho[2,1-b]thien-5-yl, thianthrenyl, such as thianthren-1-yl or thianthren-2-yl, oxanthrenyl, such as oxanthren-1-yl or oxanthren-2-yl, phenoxathiinyl, such as phenoxathiin-1-yl, phenoxathiin-2-yl, phenoxathiin-3-yl or phenoxathiin-4-yl, 9H-xanthyl, such as 9H-xanth-1-yl, 9H-xanth-2-yl, 9H-xanth-3-yl or 9H-xanth-9-yl, and 9H-thioxanthyl, such as 9H-thioxanth-1-yl, 9H-thioxanth-2-yl, 9H-thioxanth-3-yl or 9H-thioxanth-9-yl.

Even more preferably, Ar^{Y} and Ar^{A} are independently selected from phenyl, naphthyl, fluorenyl, phenanthrenyl, dibenzo[b,d]furanyl, dibenzo[b,d]thienyl, thianthrenyl, oxanthrenyl, phenoxathiinyl, 9H-xanthyl and 9H-thioxanthyl, such as phenyl, naphth-1-yl, naphth-2-yl, fluoren-2-yl, fluoren-3-yl, fluoren-9-yl, phenanthren-1-yl, phenanthren-2-yl, phenanthren-3-yl, phenanthren-4-yl, phenanthren-9-yl, dibenzo[b,d]thien-1-yl, dibenzo[b,d]thien-2-yl, dibenzo[b,d]thien-3-yl, dibenzo[b,d]thien-4-yl, dibenzo[b,d]furan-1-yl, dibenzo[b,d]furan-2-yl, dibenzo[b,d]furan-3-yl or dibenzo[b,d]furan-4-yl, thianthren-1-yl, thianthren-2-yl, oxanthren-1-yl, oxanthren-2-yl, phenoxathiin-1-yl, phenoxathiin-2-yl, phenoxathiin-3-yl, phenoxathiin-4-yl, 9H-xanth-1-yl, 9H-xanth-2-yl, 9H-xanth-3-yl, 9H-xanth-9-yl, 9H-thioxanth-1-yl, 9H-thioxanth-2-yl, 9H-thioxanth-3-yl and 9H-thioxanth-9-yl.

Particularly, Ar^{Y} and Ar^{A} are independently selected from phenyl, naphth-1-yl, naphth-2-yl, fluoren-2-yl, fluoren-9-yl, phenanthren-9-yl, dibenzo[b,d]thien-2-yl, dibenzo[b,d]thien-3-yl, dibenzo[b,d]thien-4-yl, dibenzo[b,d]furan-2-yl, dibenzo[b,d]furan-3-yl or dibenzo[b,d]furan-4-yl, thianthren-1-yl, thianthren-2-yl, oxanthren-1-yl, oxanthren-2-yl, 9H-xanth-9-yl and 9H-thioxanth-9-yl.

Specifically, Ar^{Y} and Ar^{A} are independently selected from selected from phenyl, naphth-1-yl, naphth-2-yl and phenanthren-9-yl,

Q is preferably selected from a single bond, S, O and SO₂, in particular from a single bond, S and O, and specifically is a single bond.

R¹, R², R³ and R⁴ are preferably, independently of one another, selected from the group consisting of halogen, C₂-C₃-alkynyl, CN, R, OR and CHₜR′₃₋ₜ, and more preferably from the group of fluorine, CN, R and OR, where t is 1 or 2, especially 2, and the variable R and R′ each have one of the meanings defined herein, especially a preferred one. In particular, the radicals R¹, R², R³ and R⁴ are independently selected from the group consisting of fluorine, CN, methyl, methoxy, phenyl, naphthyl and phenanthrenyl, and specifically from the group consisting of fluorine, phenyl or naphthyl.

R^{5a} and R^{5b} are preferably, independently of one another, selected from the group consisting of hydrogen, fluorine, CN, R, OR and CHₖR′₃₋ₖ, and more preferably from the group of hydrogen, fluorine, CN, R and OR, where k is 1 or 2, especially 2, and the variable R and R′ each have one of the meanings defined herein, especially a preferred one. In particular, the radicals R^{5a} and R^{5b} are independently selected from the group consisting of hydrogen, fluorine, CN, methyl, methoxy, phenyl, naphthyl and phenanthrenyl, and specifically from the group consisting of hydrogen, fluorine, phenyl or naphthyl.

R^{Ar} is preferably selected from the group consisting of R, OR and CHₜR′₃₋ₜ, and more preferably from the group of R and OR, where t is 1 or 2, especially 2, and the variables R and R′ each have one of the meanings defined herein, especially a preferred one. In particular, the radical R^{Ar} is selected from the group consisting of methyl, methoxy, phenyl, naphthyl, phenanthrenyl and triphenylenyl, and specifically is phenyl, naphthyl or phenanthrenyl.

R is preferably selected from the group consisting of methyl, ethyl, phenyl, naphthyl, phenanthrenyl and triphenylenyl, which are unsubstituted or substituted by 1, 2 or 3 identical or different radicals R′″, where R′″, independently of each occurrence, has one of the meanings defined herein, in particular a preferred one. More preferably, R is selected from the group consisting of phenyl, naphthyl and phenanthrenyl, which are unsubstituted.

R′ is preferably selected from the group consisting of phenyl, naphthyl, phenanthrenyl and triphenylenyl, which are unsubstituted or substituted by 1, 2 or 3 identical or different radicals R′″, where R′″, independently of each occurrence, has one of the meanings defined herein, in particular a preferred one. More preferably, R′ is selected from the group consisting of phenyl, naphthyl and phenanthrenyl, which are unsubstituted.

R″ is preferably selected from the group consisting of hydrogen, methyl, phenyl and naphthyl, where phenyl and naphthyl are unsubstituted or substituted by 1, 2 or 3, especially 1 or 2, identical or different radicals R′″, where R′″, independently of each occurrence, has one of the meanings defined herein, in particular a preferred one. More preferably, R″ is unsubstituted phenyl or unsubstituted naphthyl.

R′″ is preferably selected from the group consisting of phenyl, OCH₃ and CH₃.

In a particular subgroup (7a) of groups (7), (9) and (10) of embodiments, where in formula (I) the groups Y¹ and Y² are both —CH₂—, the variable n is 1, the variables m, p, q and r are all 0 and the groups X¹ and X² have identical meanings, the compound of formula (I) is a compound of the formula (Ia),

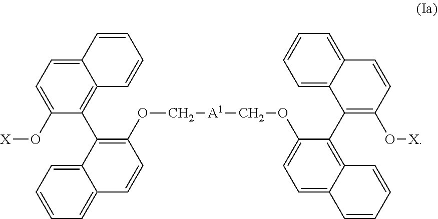

where X represents the identical groups X¹ and X², and where A¹, X¹ and X² have the meanings defined herein, in particular the meanings mentioned as preferred.

In this subgroup (7a) of groups (7), (9) and (10) of embodiments the structural unit of the formula (II) is a structural unit of the formula (IIa),

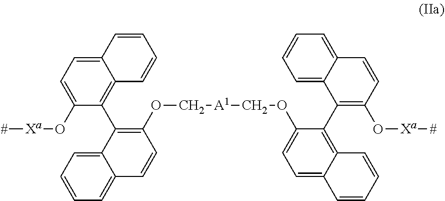

where #represents a connection point to a neighboring structural unit, where X^{a} represents the identical groups X^{1a} and X^{2a}, and where the variables A¹, X^{1a} and X^{2a} have the meanings defined herein, in particular the meanings mentioned as preferred.

Preferably, the moieties X in formula (Ia) as well as the moieties X^{a} in formula (IIa) are defined either as in one of groups (1) and (1.1) of the embodiments, in group (2) of the embodiments or in one of groups (3) and (3.1) of the embodiments. More preferably, the moieties X in formula (Ia) as well as the moieties X^{a} in formula (IIa) are defined either as in group (1.1) of the embodiments, in group (2) of the embodiments or in group (3.1) of the embodiments. Thus, the moieties X in formula (Ia) are here in particular selected from the group consisting of hydrogen, 2-hydroxyethyl (i.e. 2-(HO)ethyl), 4-(hydroxymethyl)phenyl)methyl, (3-(hydroxymethyl)phenyl)methyl, (4-(hydroxymethyl)-1-naphthyl)methyl, (5-(hydroxymethyl)-1-naphthyl)methyl, (6-(hydroxymethyl)-2-naphthyl)methyl, 4′-(hydroxymethyl)-1,1′-biphenylyl-4-methyl, methoxycarbonyl-methyl, (4-(methoxycarbonyl)phenyl)methyl, (3-(methoxycarbonyl)phenyl)methyl, (4-(methoxycarbonyl)-1-naphthyl)methyl, (5-(methoxycarbonyl)-1-naphthyl)methyl and (6-(methoxycarbonyl)-2-naphthyl)methyl, especially selected from hydrogen, 2-hydroxyethyl, methoxycarbonyl-methyl, (4-(hydroxymethyl)phenyl)methyl, (3-(hydroxymethyl)phenyl)methyl, (4-(methoxycarbonyl)phenyl)methyl and (3-(methoxycarbonyl)phenyl)methyl, and specifically selected from hydrogen, 2-hydroxyethyl, (4-(hydroxymethyl)phenyl)methyl and (3-(hydroxymethyl)phenyl)methyl. Accordingly, the moieties X^{a} in formula (IIa) are here in particular selected from the group consisting of a single bond, 2(-O)-ethyl, (4(O-methyl)phenyl)methyl, (3(-O-methyl)phenyl)methyl, (4(-O-methyl)-1-naphthyl)methyl, (5(-O-methyl)-1-naphthyl)methyl, (6(-O-methyl)-2-naphthyl)methyl, 4′(-O-methyl)-1,1′-biphenylyl-4-methyl, —O—C(O)-methyl, (4(-O—C(O)-phenyl)methyl, (3(-O—C(O)-phenyl)methyl, (4-(-O—C(O)-)-1-naphthyl)methyl, (5-(-O—C(O)-)-1-naphthyl)methyl and (6-(-O—C(O)-)-2-naphthyl)methyl, especially selected from a single bond, 2(-O)-ethyl, —O—C(O)-methyl, (4(-O-methyl)phenyl)methyl, (3(-O-methyl)phenyl)methyl, 4(-O—C(O)-phenyl)methyl and (3-(-O—C(O)-phenyl)methyl, and specifically selected from a single bond, 2(-O)-ethyl, (4(-O-methyl)phenyl)methyl and (3(-O-methyl)phenyl)methyl.

Preference is also given to compounds of the formula (Ia) and to structural units of the formula (IIa), where the moiety A¹ is defined either as in one of groups (5) and (5.1) of embodiments or in one of groups (6), (6.1), (6.2) (6.3), and (6.4) of embodiments. More preferably, the moiety X in formula (Ia) as well as in formula (IIa) are defined either as in group (5.1) of the embodiments, or in group (6.4) of the embodiments. Thus, the moieties X in formula (Ia) are here in particular selected from the group consisting of bisphenylmethandiyl, bis(naphth-1-yl)methandiyl, bis(naphth-2-yl)methandiyl, bis(phenanthren-9-yl)methandiyl, 1,4-phenylene, 1,2-phenylene, 1,3-phenylene, 2,3-naphthylene, 2,7-naphthylene, 2,6-naphthylene, 1,4-naphthylene, 1,5-naphthylene, 1,8-naphthylene, 4,6-dibenzo[b,a]thienylene, 2,8-dibenzo[b,d]thienylene, 3,3′-biphenylylene, 4,4′-biphenylylene, 9,9-9H-fluorenylene, 2,7-9H-fluorenylene, 2,7-thianthrenylene, 2,8-thianthrenylene, 1,4-thianthrenylene, 2,3-thianthrenylene, 1,6-thianthrenylene, 1,9-thianthrenylene, 9,9-9H-xanthylene and 9,9-9H-thioxanthylene.

Examples of the particular subgroup (7a) are the compounds of the formula (Ia) and the structural units of formula (IIa), in which the combination of the moieties X or moieties X^{a}, respectively, and the variable A¹ is as defined in any one of the lines 1 to 266 in table A below, where X^{a} in each case is derived from X in formula (Ia), if X is hydrogen, by replacing hydrogen with a single bond, or, if X is not hydrogen, by replacing the —OH or —OR^{x} group of X with an oxo (—O—) unit, where X has one of the meanings defined herein, especially one of those mentioned herein as preferred.

TABLE A

| # | X | A¹ |
|---|---|---|
| 1 | hydrogen | 1,4-phenylene |
| 2 | hydrogen | 1,2-phenylene |
| 3 | hydrogen | 1,3-phenylene |
| 4 | hydrogen | 2,3-naphthylene |
| 5 | hydrogen | 2,7-naphthylene |
| 6 | hydrogen | 2,6-naphthylene |
| 7 | hydrogen | 1,4-naphthylene |
| 8 | hydrogen | 1,5-naphthylene |
| 9 | hydrogen | 1,8-naphthylene |
| 10 | hydrogen | 4,6-dibenzo[b,d]thienylene |
| 11 | hydrogen | 2,8-dibenzo[b,d]thienylene |
| 12 | hydrogen | 3,7-dibenzo[b,d]thienylene |
| 13 | hydrogen | 3,3′-biphenylylene |
| 14 | hydrogen | 4,4′-biphenylylene |
| 15 | hydrogen | 9,9-9H-fluorenylene |
| 16 | hydrogen | 2,7-9H-fluorenylene |
| 17 | hydrogen | 2,7-thianthrenylene |
| 18 | hydrogen | 2,8-thianthrenylene |
| 19 | hydrogen | 1,4-thianthrenylene |
| 20 | hydrogen | 2,3-thianthrenylene |
| 21 | hydrogen | 1,6-thianthrenylene |
| 22 | hydrogen | 1,9-thianthrenylene |
| 23 | hydrogen | 9,9-9H-xanthylene |
| 24 | hydrogen | 9,9-9H-thioxanthylene |
| 25 | 2-hydroxyethyl | 1,4-phenylene |
| 26 | 2-hydroxyethyl | 1,2-phenylene |
| 27 | 2-hydroxyethyl | 1,3-phenylene |
| 28 | 2-hydroxyethyl | 2,3-naphthylene |
| 29 | 2-hydroxyethyl | 2,7-naphthylene |
| 30 | 2-hydroxyethyl | 2,6-naphthylene |
| 31 | 2-hydroxyethyl | 1,4-naphthylene |
| 32 | 2-hydroxyethyl | 1,5-naphthylene |
| 33 | 2-hydroxyethyl | 1,8-naphthylene |
| 34 | 2-hydroxyethyl | 4,6-dibenzo[b,d]thienylene |
| 35 | 2-hydroxyethyl | 2,8-dibenzo[b,d]thienylene |
| 36 | 2-hydroxyethyl | 3,7-dibenzo[b,d]thienylene |
| 37 | 2-hydroxyethyl | 3,3′-biphenylylene |
| 38 | 2-hydroxyethyl | 4,4′-biphenylylene |
| 39 | 2-hydroxyethyl | 9,9-9H-fluorenylene |
| 40 | 2-hydroxyethyl | 2,7-9H-fluorenylene |
| 41 | 2-hydroxyethyl | 2,7-thianthrenylene |
| 42 | 2-hydroxyethyl | 2,8-thianthrenylene |
| 43 | 2-hydroxyethyl | 1,4-thianthrenylene |
| 44 | 2-hydroxyethyl | 2,3-thianthrenylene |
| 45 | 2-hydroxyethyl | 1,6-thianthrenylene |
| 46 | 2-hydroxyethyl | 1,9-thianthrenylene |
| 47 | 2-hydroxyethyl | 9,9-9H-xanthylene |
| 48 | 2-hydroxyethyl | 9,9-9H-thioxanthylene |
| 49 | methoxycarbonyl-methyl | 1,4-phenylene |
| 50 | methoxycarbonyl-methyl | 1,2-phenylene |
| 51 | methoxycarbonyl-methyl | 1,3-phenylene |
| 52 | methoxycarbonyl-methyl | 2,3-naphthylene |
| 53 | methoxycarbonyl-methyl | 2,7-naphthylene |
| 54 | methoxycarbonyl-methyl | 2,6-naphthylene |
| 55 | methoxycarbonyl-methyl | 1,4-naphthylene |
| 56 | methoxycarbonyl-methyl | 1,5-naphthylene |
| 57 | methoxycarbonyl-methyl | 1,8-naphthylene |
| 58 | methoxycarbonyl-methyl | 4,6-dibenzo[b,d]thienylene |
| 59 | methoxycarbonyl-methyl | 2,8-dibenzo[b,d]thienylene |
| 60 | methoxycarbonyl-methyl | 3,7-dibenzo[b,d]thienylene |
| 61 | methoxycarbonyl-methyl | 3,3′-biphenylylene |
| 62 | methoxycarbonyl-methyl | 4,4′-biphenylylene |
| 63 | methoxycarbonyl-methyl | 9,9-9H-fluorenylene |
| 64 | methoxycarbonyl-methyl | 2,7-9H-fluorenylene |
| 65 | methoxycarbonyl-methyl | 2,7-thianthrenylene |
| 66 | methoxycarbonyl-methyl | 2,8-thianthrenylene |
| 67 | methoxycarbonyl-methyl | 1,4-thianthrenylene |
| 68 | methoxycarbonyl-methyl | 2,3-thianthrenylene |
| 69 | methoxycarbonyl-methyl | 1,6-thianthrenylene |
| 70 | methoxycarbonyl-methyl | 1,9-thianthrenylene |
| 71 | methoxycarbonyl-methyl | 9,9-9H-xanthylene |
| 72 | methoxycarbonyl-methyl | 9,9-9H-thioxanthylene |
| 73 | (4-(hydroxymethyl)phenyl)methyl | 1,4-phenylene |
| 74 | (4-(hydroxymethyl)phenyl)methyl | 1,2-phenylene |
| 75 | (4-(hydroxymethyl)phenyl)methyl | 1,3-phenylene |
| 76 | (4-(hydroxymethyl)phenyl)methyl | 2,3-naphthylene |
| 77 | (4-(hydroxymethyl)phenyl)methyl | 2,7-naphthylene |
| 78 | (4-(hydroxymethyl)phenyl)methyl | 2,6-naphthylene |
| 79 | (4-(hydroxymethyl)phenyl)methyl | 1,4-naphthylene |
| 80 | (4-(hydroxymethyl)phenyl)methyl | 1,5-naphthylene |
| 81 | (4-(hydroxymethyl)phenyl)methyl | 1,8-naphthylene |
| 82 | (4-(hydroxymethyl)phenyl)methyl | 4,6-dibenzo[b,d]thienylene |
| 83 | (4-(hydroxymethyl)phenyl)methyl | 2,8-dibenzo[b,d]thienylene |
| 84 | (4-(hydroxymethyl)phenyl)methyl | 3,7-dibenzo[b,d]thienylene |
| 85 | (4-(hydroxymethyl)phenyl)methyl | 3,3′-biphenylylene |
| 86 | (4-(hydroxymethyl)phenyl)methyl | 4,4′-biphenylylene |
| 87 | (4-(hydroxymethyl)phenyl)methyl | 9,9-9H-fluorenylene |
| 88 | (4-(hydroxymethyl)phenyl)methyl | 2,7-9H-fluorenylene |
| 89 | (4-(hydroxymethyl)phenyl)methyl | 2,7-thianthrenylene |
| 90 | (4-(hydroxymethyl)phenyl)methyl | 2,8-thianthrenylene |
| 91 | (4-(hydroxymethyl)phenyl)methyl | 1,4-thianthrenylene |
| 92 | (4-(hydroxymethyl)phenyl)methyl | 2,3-thianthrenylene |
| 93 | (4-(hydroxymethyl)phenyl)methyl | 1,6-thianthrenylene |
| 94 | (4-(hydroxymethyl)phenyl)methyl | 1,9-thianthrenylene |
| 95 | (4-(hydroxymethyl)phenyl)methyl | 9,9-9H-xanthylene |
| 96 | (4-(hydroxymethyl)phenyl)methyl | 9,9-9H-thioxanthylene |
| 97 | (3-(hydroxymethyl)phenyl)methyl | 1,4-phenylene |
| 98 | (3-(hydroxymethyl)phenyl)methyl | 1,2-phenylene |
| 99 | (3-(hydroxymethyl)phenyl)methyl | 1,3-phenylene |
| 100 | (3-(hydroxymethyl)phenyl)methyl | 2,3-naphthylene |
| 101 | (3-(hydroxymethyl)phenyl)methyl | 2,7-naphthylene |
| 102 | (3-(hydroxymethyl)phenyl)methyl | 2,6-naphthylene |
| 103 | (3-(hydroxymethyl)phenyl)methyl | 1,4-naphthylene |
| 104 | (3-(hydroxymethyl)phenyl)methyl | 1,5-naphthylene |
| 105 | (3-(hydroxymethyl)phenyl)methyl | 1,8-naphthylene |
| 106 | (3-(hydroxymethyl)phenyl)methyl | 4,6-dibenzo[b,d]thienylene |
| 107 | (3-(hydroxymethyl)phenyl)methyl | 2,8-dibenzo[b,d]thienylene |
| 108 | (3-(hydroxymethyl)phenyl)methyl | 3,7-dibenzo[b,d]thienylene |
| 109 | (3-(hydroxymethyl)phenyl)methyl | 3,3′-biphenylylene |
| 110 | (3-(hydroxymethyl)phenyl)methyl | 4,4′-biphenylylene |
| 111 | (3-(hydroxymethyl)phenyl)methyl | 9,9-9H-fluorenylene |
| 112 | (3-(hydroxymethyl)phenyl)methyl | 2,7-9H-fluorenylene |
| 113 | (3-(hydroxymethyl)phenyl)methyl | 2,7-thianthrenylene |
| 114 | (3-(hydroxymethyl)phenyl)methyl | 2,8-thianthrenylene |
| 115 | (3-(hydroxymethyl)phenyl)methyl | 1,4-thianthrenylene |
| 116 | (3-(hydroxymethyl)phenyl)methyl | 2,3-thianthrenylene |
| 117 | (3-(hydroxymethyl)phenyl)methyl | 1,6-thianthrenylene |
| 118 | (3-(hydroxymethyl)phenyl)methyl | 1,9-thianthrenylene |
| 119 | (3-(hydroxymethyl)phenyl)methyl | 9,9-9H-xanthylene |
| 120 | (3-(hydroxymethyl)phenyl)methyl | 9,9-9H-thioxanthylene |
| 121 | (4-(hydroxymethyl)-1-naphthyl)methyl | 1,4-phenylene |
| 122 | (4-(hydroxymethyl)-1-naphthyl)methyl | 1,2-phenylene |
| 123 | (4-(hydroxymethyl)-1-naphthyl)methyl | 1,3-phenylene |
| 124 | (4-(hydroxymethyl)-1-naphthyl)methyl | 2,3-naphthylene |
| 125 | (4-(hydroxymethyl)-1-naphthyl)methyl | 2,7-naphthylene |
| 126 | (4-(hydroxymethyl)-1-naphthyl)methyl | 2,6-naphthylene |
| 127 | (4-(hydroxymethyl)-1-naphthyl)methyl | 1,4-naphthylene |
| 128 | (4-(hydroxymethyl)-1-naphthyl)methyl | 1,5-naphthylene |
| 129 | (4-(hydroxymethyl)-1-naphthyl)methyl | 1,8-naphthylene |
| 130 | (4-(hydroxymethyl)-1-naphthyl)methyl | 4,6-dibenzo[b,d]thienylene |
| 131 | (4-(hydroxymethyl)-1-naphthyl)methyl | 2,8-dibenzo[b,d]thienylene |
| 132 | (4-(hydroxymethyl)-1-naphthyl)methyl | 3,7-dibenzo[b,d]thienylene |
| 133 | (4-(hydroxymethyl)-1-naphthyl)methyl | 3,3′-biphenylylene |
| 134 | (4-(hydroxymethyl)-1-naphthyl)methyl | 4,4′-biphenylylene |
| 135 | (4-(hydroxymethyl)-1-naphthyl)methyl | 9,9-9H-fluorenylene |
| 136 | (4-(hydroxymethyl)-1-naphthyl)methyl | 2,7-9H-fluorenylene |
| 137 | (4-(hydroxymethyl)-1-naphthyl)methyl | 2,7-thianthrenylene |
| 138 | (4-(hydroxymethyl)-1-naphthyl)methyl | 2,8-thianthrenylene |
| 139 | (4-(hydroxymethyl)-1-naphthyl)methyl | 1,4-thianthrenylene |
| 140 | (4-(hydroxymethyl)-1-naphthyl)methyl | 2,3-thianthrenylene |
| 141 | (4-(hydroxymethyl)-1-naphthyl)methyl | 1,6-thianthrenylene |
| 142 | (4-(hydroxymethyl)-1-naphthyl)methyl | 1,9-thianthrenylene |
| 143 | (4-(hydroxymethyl)-1-naphthyl)methyl | 9,9-9H-xanthylene |
| 144 | (4-(hydroxymethyl)-1-naphthyl)methyl | 9,9-9H-thioxanthylene |
| 145 | 6-(hydroxymethyl)-2-naphthyl)methyl | 1,4-phenylene |
| 146 | 6-(hydroxymethyl)-2-naphthyl)methyl | 1,2-phenylene |
| 147 | 6-(hydroxymethyl)-2-naphthyl)methyl | 1,3-phenylene |
| 148 | 6-(hydroxymethyl)-2-naphthyl)methyl | 2,3-naphthylene |
| 149 | 6-(hydroxymethyl)-2-naphthyl)methyl | 2,7-naphthylene |
| 150 | 6-(hydroxymethyl)-2-naphthyl)methyl | 2,6-naphthylene |
| 151 | 6-(hydroxymethyl)-2-naphthyl)methyl | 1,4-naphthylene |
| 152 | 6-(hydroxymethyl)-2-naphthyl)methyl | 1,5-naphthylene |
| 153 | 6-(hydroxymethyl)-2-naphthyl)methyl | 1,8-naphthylene |
| 154 | 6-(hydroxymethyl)-2-naphthyl)methyl | 4,6-dibenzo[b,d]thienylene |
| 155 | 6-(hydroxymethyl)-2-naphthyl)methyl | 2,8-dibenzo[b,d]thienylene |
| 156 | 6-(hydroxymethyl)-2-naphthyl)methyl | 3,7-dibenzo[b,d]thienylene |
| 157 | 6-(hydroxymethyl)-2-naphthyl)methyl | 3,3′-biphenylylene |
| 158 | 6-(hydroxymethyl)-2-naphthyl)methyl | 4,4′-biphenylylene |
| 159 | 6-(hydroxymethyl)-2-naphthyl)methyl | 9,9-9H-fluorenylene |
| 160 | 6-(hydroxymethyl)-2-naphthyl)methyl | 2,7-9H-fluorenylene |
| 161 | 6-(hydroxymethyl)-2-naphthyl)methyl | 2,7-thianthrenylene |
| 162 | 6-(hydroxymethyl)-2-naphthyl)methyl | 2,8-thianthrenylene |
| 163 | 6-(hydroxymethyl)-2-naphthyl)methyl | 1,4-thianthrenylene |
| 164 | 6-(hydroxymethyl)-2-naphthyl)methyl | 2,3-thianthrenylene |
| 165 | 6-(hydroxymethyl)-2-naphthyl)methyl | 1,6-thianthrenylene |
| 166 | 6-(hydroxymethyl)-2-naphthyl)methyl | 1,9-thianthrenylene |
| 167 | 6-(hydroxymethyl)-2-naphthyl)methyl | 9,9-9H-xanthylene |
| 168 | 6-(hydroxymethyl)-2-naphthyl)methyl | 9,9-9H-thioxanthylene |
| 169 | (4-(methoxycarbonyl)phenyl)methyl | 1,4-phenylene |
| 170 | (4-(methoxycarbonyl)phenyl)methyl | 1,2-phenylene |
| 171 | (4-(methoxycarbonyl)phenyl)methyl | 1,3-phenylene |
| 172 | (4-(methoxycarbonyl)phenyl)methyl | 2,3-naphthylene |
| 173 | (4-(methoxycarbonyl)phenyl)methyl | 2,7-naphthylene |
| 174 | (4-(methoxycarbonyl)phenyl)methyl | 2,6-naphthylene |
| 175 | (4-(methoxycarbonyl)phenyl)methyl | 1,4-naphthylene |
| 176 | (4-(methoxycarbonyl)phenyl)methyl | 1,5-naphthylene |
| 177 | (4-(methoxycarbonyl)phenyl)methyl | 1,8-naphthylene |
| 178 | (4-(methoxycarbonyl)phenyl)methyl | 4,6-dibenzo[b,d]thienylene |
| 179 | (4-(methoxycarbonyl)phenyl)methyl | 2,8-dibenzo[b,d]thienylene |
| 180 | (4-(methoxycarbonyl)phenyl)methyl | 3,7-dibenzo[b,d]thienylene |
| 181 | (4-(methoxycarbonyl)phenyl)methyl | 3,3′-biphenylylene |
| 182 | (4-(methoxycarbonyl)phenyl)methyl | 4,4′-biphenylylene |
| 183 | (4-(methoxycarbonyl)phenyl)methyl | 9,9-9H-fluorenylene |
| 184 | (4-(methoxycarbonyl)phenyl)methyl | 2,7-9H-fluorenylene |
| 185 | (4-(methoxycarbonyl)phenyl)methyl | 2,7-thianthrenylene |
| 186 | (4-(methoxycarbonyl)phenyl)methyl | 2,8-thianthrenylene |
| 187 | (4-(methoxycarbonyl)phenyl)methyl | 1,4-thianthrenylene |
| 188 | (4-(methoxycarbonyl)phenyl)methyl | 2,3-thianthrenylene |
| 189 | (4-(methoxycarbonyl)phenyl)methyl | 1,6-thianthrenylene |
| 190 | (4-(methoxycarbonyl)phenyl)methyl | 1,9-thianthrenylene |
| 191 | (4-(methoxycarbonyl)phenyl)methyl | 9,9-9H-xanthylene |
| 192 | (4-(methoxycarbonyl)phenyl)methyl | 9,9-9H-thioxanthylene |
| 193 | (3-(methoxycarbonyl)phenyl)methyl | 1,4-phenylene |
| 194 | (3-(methoxycarbonyl)phenyl)methyl | 1,2-phenylene |
| 195 | (3-(methoxycarbonyl)phenyl)methyl | 1,3-phenylene |
| 196 | (3-(methoxycarbonyl)phenyl)methyl | 2,3-naphthylene |
| 197 | (3-(methoxycarbonyl)phenyl)methyl | 2,7-naphthylene |
| 198 | (3-(methoxycarbonyl)phenyl)methyl | 2,6-naphthylene |
| 199 | (3-(methoxycarbonyl)phenyl)methyl | 1,4-naphthylene |
| 200 | (3-(methoxycarbonyl)phenyl)methyl | 1,5-naphthylene |
| 201 | (3-(methoxycarbonyl)phenyl)methyl | 1,8-naphthylene |
| 202 | (3-(methoxycarbonyl)phenyl)methyl | 4,6-dibenzo[b,d]thienylene |
| 203 | (3-(methoxycarbonyl)phenyl)methyl | 2,8-dibenzo[b,d]thienylene |
| 204 | (3-(methoxycarbonyl)phenyl)methyl | 3,7-dibenzo[b,d]thienylene |
| 205 | (3-(methoxycarbonyl)phenyl)methyl | 3,3′-biphenylylene |
| 206 | (3-(methoxycarbonyl)phenyl)methyl | 4,4′-biphenylylene |
| 207 | (3-(methoxycarbonyl)phenyl)methyl | 9,9-9H-fluorenylene |
| 208 | (3-(methoxycarbonyl)phenyl)methyl | 2,7-9H-fluorenylene |
| 209 | (3-(methoxycarbonyl)phenyl)methyl | 2,7-thianthrenylene |
| 210 | (3-(methoxycarbonyl)phenyl)methyl | 2,8-thianthrenylene |
| 211 | (3-(methoxycarbonyl)phenyl)methyl | 1,4-thianthrenylene |
| 212 | (3-(methoxycarbonyl)phenyl)methyl | 2,3-thianthrenylene |
| 213 | (3-(methoxycarbonyl)phenyl)methyl | 1,6-thianthrenylene |
| 214 | (3-(methoxycarbonyl)phenyl)methyl | 1,9-thianthrenylene |
| 215 | (3-(methoxycarbonyl)phenyl)methyl | 9,9-9H-xanthylene |
| 216 | (3-(methoxycarbonyl)phenyl)methyl | 9,9-9H-thioxanthylene |
| 217 | (4-(methoxycarbonyl)-1-naphthyl)methyl | 1,4-phenylene |
| 218 | (4-(methoxycarbonyl)-1-naphthyl)methyl | 1,2-phenylene |
| 219 | (4-(methoxycarbonyl)-1-naphthyl)methyl | 1,3-phenylene |
| 220 | (4-(methoxycarbonyl)-1-naphthyl)methyl | 2,3-naphthylene |
| 221 | (4-(methoxycarbonyl)-1-naphthyl)methyl | 2,7-naphthylene |
| 222 | (4-(methoxycarbonyl)-1-naphthyl)methyl | 2,6-naphthylene |
| 223 | (4-(methoxycarbonyl)-1-naphthyl)methyl | 1,4-naphthylene |
| 224 | (4-(methoxycarbonyl)-1-naphthyl)methyl | 1,5-naphthylene |
| 225 | (4-(methoxycarbonyl)-1-naphthyl)methyl | 1,8-naphthylene |
| 226 | (4-(methoxycarbonyl)-1-naphthyl)methyl | 4,6-dibenzo[b,d]thienylene |
| 227 | (4-(methoxycarbonyl)-1-naphthyl)methyl | 2,8-dibenzo[b,d]thienylene |
| 228 | (4-(methoxycarbonyl)-1-naphthyl)methyl | 3,7-dibenzo[b,d]thienylene |
| 229 | (4-(methoxycarbonyl)-1-naphthyl)methyl | 3,3′-biphenylylene |
| 230 | (4-(methoxycarbonyl)-1-naphthyl)methyl | 4,4′-biphenylylene |
| 231 | (4-(methoxycarbonyl)-1-naphthyl)methyl | 9,9-9H-fluorenylene |
| 232 | (4-(methoxycarbonyl)-1-naphthyl)methyl | 2,7-9H-fluorenylene |
| 233 | (4-(methoxycarbonyl)-1-naphthyl)methyl | 2,7-thianthrenylene |
| 234 | (4-(methoxycarbonyl)-1-naphthyl)methyl | 2,8-thianthrenylene |
| 235 | (4-(methoxycarbonyl)-1-naphthyl)methyl | 1,4-thianthrenylene |
| 236 | (4-(methoxycarbonyl)-1-naphthyl)methyl | 2,3-thianthrenylene |
| 237 | (4-(methoxycarbonyl)-1-naphthyl)methyl | 1,6-thianthrenylene |
| 238 | (4-(methoxycarbonyl)-1-naphthyl)methyl | 1,9-thianthrenylene |
| 239 | (4-(methoxycarbonyl)-1-naphthyl)methyl | 9,9-9H-xanthylene |
| 240 | (4-(methoxycarbonyl)-1-naphthyl)methyl | 9,9-9H-thioxanthylene |
| 241 | (6-(methoxycarbonyl)-2-naphthyl)methyl | 1,4-phenylene |
| 242 | (6-(methoxycarbonyl)-2-naphthyl)methyl | 1,2-phenylene |
| 243 | (6-(methoxycarbonyl)-2-naphthyl)methyl | 1,3-phenylene |
| 244 | (6-(methoxycarbonyl)-2-naphthyl)methyl | 2,3-naphthylene |
| 245 | (6-(methoxycarbonyl)-2-naphthyl)methyl | 2,7-naphthylene |
| 246 | (6-(methoxycarbonyl)-2-naphthyl)methyl | 2,6-naphthylene |
| 247 | (6-(methoxycarbonyl)-2-naphthyl)methyl | 1,4-naphthylene |
| 248 | (6-(methoxycarbonyl)-2-naphthyl)methyl | 1,5-naphthylene |
| 249 | (6-(methoxycarbonyl)-2-naphthyl)methyl | 1,8-naphthylene |
| 250 | (6-(methoxycarbonyl)-2-naphthyl)methyl | 4,6-dibenzo[b,d]thienylene |
| 251 | (6-(methoxycarbonyl)-2-naphthyl)methyl | 2,8-dibenzo[b,d]thienylene |
| 252 | (6-(methoxycarbonyl)-2-naphthyl)methyl | 3,7-dibenzo[b,d]thienylene |
| 253 | (6-(methoxycarbonyl)-2-naphthyl)methyl | 3,3′-biphenylylene |
| 254 | (6-(methoxycarbonyl)-2-naphthyl)methyl | 4,4′-biphenylylene |
| 255 | (6-(methoxycarbonyl)-2-naphthyl)methyl | 9,9-9H-fluorenylene |
| 256 | (6-(methoxycarbonyl)-2-naphthyl)methyl | 2,7-9H-fluorenylene |
| 257 | (6-(methoxycarbonyl)-2-naphthyl)methyl | 2,7-thianthrenylene |
| 258 | (6-(methoxycarbonyl)-2-naphthyl)methyl | 2,8-thianthrenylene |
| 259 | (6-(methoxycarbonyl)-2-naphthyl)methyl | 1,4-thianthrenylene |
| 260 | (6-(methoxycarbonyl)-2-naphthyl)methyl | 2,3-thianthrenylene |
| 261 | (6-(methoxycarbonyl)-2-naphthyl)methyl | 1,6-thianthrenylene |
| 262 | (6-(methoxycarbonyl)-2-naphthyl)methyl | 1,9-thianthrenylene |
| 263 | (6-(methoxycarbonyl)-2-naphthyl)methyl | 9,9-9H-xanthylene |
| 264 | (6-(methoxycarbonyl)-2-naphthyl)methyl | 9,9-9H-thioxanthylene |
| 265 | 4′-(hydroxymethyl)-1,1′-biphenylyl-4-methyl | 1,4-phenylene |
| 266 | 2-hydroxyethyl | bis[(naphth-1-yl)methyl]- |
| | | methylene |

Amongst the compounds of formula (Ia) recited in table A, particular preference is given to those compounds of formula (Ia), where the moieties X are 2-hydroxyethyl (i.e. 2-HO-ethyl), and accordingly to the structural units of formula (IIa), where the moieties X^{a} are 2(-O)-ethyl. In other words, particular preference is given to the following compounds of the formula (Ia):
2,2′-[1,4-phenylenebis(methyleneoxy[1,1′-binaphthalene]-2′,2-diyloxy)]di(ethan-1-ol)
2,2′-[1,2-phenylenebis(methyleneoxy[1,1′-binaphthalene]-2′,2-diyloxy)]di(ethan-1-ol)
2,2′-[1,3-phenylenebis(methyleneoxy[1,1′-binaphthalene]-2′,2-diyloxy)]di(ethan-1-ol)
2,2′-[[1,1′-biphenyl]-4,4′-diylbis(methyleneoxy[1,1′-binaphthalene]-2′,2-diyloxy)]-di(ethan-1-ol)
2,2′-[[1,1′-biphenyl]-3,3′-diylbis(methyleneoxy[1,1′-binaphthalene]-2′,2-diyloxy)]-di(ethan-1-ol)
2,2′-[naphthalene-2,3-diylbis(methyleneoxy[1,1′-binaphthalene]-2′,2-diyloxy)]-di(ethan-1-ol)
2,2′-[naphthalene-2,7-diylbis(methyleneoxy[1,1′-binaphthalene]-2′,2-diyloxy)]-di(ethan-1-ol)
2,2′-[naphthalene-2,6-diylbis(methyleneoxy[1,1′-binaphthalene]-2′,2-diyloxy)]-di(ethan-1-ol)
2,2′-[naphthalene-1,4-diylbis(methyleneoxy[1,1′-binaphthalene]-2′,2-diyloxy)]-di(ethan-1-ol)
2,2′-[naphthalene-1,5-diylbis(methyleneoxy[1,1′-binaphthalene]-2′,2-diyloxy)]-di(ethan-1-ol)
2,2′-[naphthalene-1,8-diylbis(methyleneoxy[1,1′-binaphthalene]-2′,2-diyloxy)]-di(ethan-1-ol)
2,2′-[dibenzo[b,d]thiene-4,6-diylbis(methyleneoxy[1,1′-binaphthalene]-2′,2-diyloxy)]-di(ethan-1-ol)
2,2′-[dibenzo[b,d]thiene-2,8-diylbis(methyleneoxy[1,1′-binaphthalene]-2′,2-diyloxy)]-di(ethan-1-ol
2,2′-[dibenzo[b,d]thiene-3,7-diylbis(methyleneoxy[1,1′-binaphthalene]-2′,2-diyloxy)]-di(ethan-1-ol
2,2′-[thianthrene-2,7-diylbis(methyleneoxy[1,1′-binaphthalene]-2′,2-diyloxy)]di(ethan-1-ol)
2,2′-[thianthrene-2,8-diylbis(methyleneoxy[1,1′-binaphthalene]-2′,2-diyloxy)]di(ethan-1-01)
2,2′-[thianthrene-1,4-diylbis(methyleneoxy[1,1′-binaphthalene]-2′,2-diyloxy)]di(ethan-1-01)
2,2′-[thianthrene-2,3-diylbis(methyleneoxy[1,1′-binaphthalene]-2′,2-diyloxy)]di(ethan-1-01)
2,2′-[thianthrene-1,6-diylbis(methyleneoxy[1,1′-binaphthalene]-2′,2-diyloxy)]di(ethan-1-ol)
2,2′-[thianthrene-1,9-diylbis(methyleneoxy[1,1′-binaphthalene]-2′,2-diyloxy)]di(ethan-1-01)
2,2′-[9H-fluorene-9,9-diylbis(methyleneoxy[1,1′-binaphthalene]-2′,2-diyloxy)]-di(ethan-1-ol)
2,2′-[9H-xanthene-9,9-diylbis(methyleneoxy[1,1′-binaphthalene]-2′,2-diyloxy)]-di(ethan-1-ol)
2,2′-[9H-thioxanthene-9,9-diylbis(methyleneoxy[1,1′-binaphthalene]-2′,2-diyloxy)]-di(ethan-1-ol)
2,2′-[9H-fluorene-2,7-diylbis(methyleneoxy[1,1′-binaphthalene]-2′,2-diyloxy)]-di(ethan-1-ol)
2,2′-[{2,2-bis[(naphthalen-1-yl)methyl]propane-1,3-diyl}bis(oxy[1,1′-binaphthalene]-2′,2-diyloxy)]di(ethan-1-ol)

Amongst the compounds of formula (Ia) recited in table A, particular preference is also given to those compounds of formula (Ia), where the moieties X are (4-(hydroxymethyl)phenyl)methyl, and accordingly to the structural units of formula (IIa), where the moieties X^{a} are (4(-O-methyl)phenyl)methyl. In other words, particular preference is given to the following compounds of the formula (Ia):
[1,4-phenylenebis(methyleneoxy[1,1′-binaphthalene]-2′,2-diyloxymethylene-4,1-phenylene)]dimethanol
[1,3-phenylenebis(methyleneoxy[1,1′-binaphthalene]-2′,2-diyloxymethylene-4,1-phenylene)]dimethanol
[1,2-phenylenebis(methyleneoxy[1,1′-binaphthalene]-2′,2-diyloxymethylene-4,1-phenylene)]dimethanol
[[1,1′-biphenyl]-4,4′-diylbis(methyleneoxy[1,1′-binaphthalene]-2′,2-diyloxymethylene-4,1-phenylene)]dimethanol
[[1,1′-biphenyl]-3,3′-diylbis(methyleneoxy[1,1′-binaphthalene]-2′,2-diyloxymethylene-4,1-phenylene)]dimethanol
[naphthalene-2,3-diylbis(methyleneoxy[1,1′-binaphthalene]-2′,2-diyloxymethylene-4,1-phenylene)]dimethanol
[naphthalene-2,7-diylbis(methyleneoxy[1,1′-binaphthalene]-2′,2-diyloxymethylene-4,1-phenylene)]dimethanol
[naphthalene-2,6-diylbis(methyleneoxy[1,1′-binaphthalene]-2′,2-diyloxymethylene-4,1-phenylene)]dimethanol
[naphthalene-1,4-diylbis(methyleneoxy[1,1′-binaphthalene]-2′,2-diyloxymethylene-4,1-phenylene)]dimethanol
[naphthalene-1,5-diylbis(methyleneoxy[1,1′-binaphthalene]-2′,2-diyloxymethylene-4,1-phenylene)]dimethanol
[naphthalene-1,8-diylbis(methyleneoxy[1,1′-binaphthalene]-2′,2-diyloxymethylene-4,1-phenylene)]dimethanol
[dibenzo[b,d]thiene-4,6-diylbis(methyleneoxy[1,1′-binaphthalene]-2′,2-diyloxymethylene-4,1-phenylene)]dimethanol
[dibenzo[b,d]thiene-2,8-diylbis(methyleneoxy[1,1′-binaphthalene]-2′,2-diyloxymethylene-4,1-phenylene)]dimethanol
[dibenzo[b,d]thiene-3,7-diylbis(methyleneoxy[1,1′-binaphthalene]-2′,2-diyloxymethylene-4,1-phenylene)]dimethanol
[thianthrene-2,7-diylbis(methyleneoxy[1,1′-binaphthalene]-2′,2-diyloxymethylene-4,1-phenylene)]dimethanol
[thianthrene-2,8-diylbis(methyleneoxy[1,1′-binaphthalene]-2′,2-diyloxymethylene-4,1-phenylene)]dimethanol
[thianthrene-1,4-diylbis(methyleneoxy[1,1′-binaphthalene]-2′,2-diyloxymethylene-4,1-phenylene)]dimethanol
[thianthrene-2,3-diylbis(methyleneoxy[1,1′-binaphthalene]-2′,2-diyloxymethylene-4,1-phenylene)]dimethanol
[thianthrene-1,6-diylbis(methyleneoxy[1,1′-binaphthalene]-2′,2-diyloxymethylene-4,1-phenylene)]dimethanol
[thianthrene-1,9-diylbis(methyleneoxy[1,1′-binaphthalene]-2′,2-diyloxymethylene-4,1-phenylene)]dimethanol
[9H-fluorene-9,9-diylbis(methyleneoxy[1,1′-binaphthalene]-2′,2-diyloxymethylene-4,1-phenylene)]dimethanol
[9H-fluorene-2,7-diylbis(methyleneoxy[1,1′-binaphthalene]-2′,2-diyloxymethylene-4,1-phenylene)]dimethanol
[9H-xanthene-9,9-diylbis(methyleneoxy[1,1′-binaphthalene]-2′,2-diyloxymethylene-4,1-phenylene)]dimethanol
[9H-thioxanthene-9,9-diylbis(methyleneoxy[1,1′-binaphthalene]-2′,2-diyloxymethylene-4,1-phenylene)]dimethanol

In a particular subgroup (8.1a) of groups (8.1), (9) and (10) of embodiments, where in formula (I) the moiety —Y¹-A¹-Y²— is —CH₂—, the variable n is 1, the variables m, p, q and r are all 0 and the groups X¹ and X² have identical meanings, the compound of formula (I) is a compound of the formula (Ib),

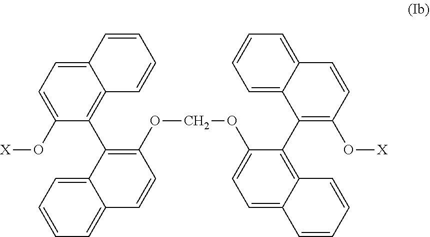

where X represents the identical groups X¹ and X², and where X¹ and X² have the meanings defined herein, in particular the meanings mentioned as preferred.

In this subgroup (8.1a) of groups (8.1), (9) and (10) of embodiments the structural unit of the formula (II) is a structural unit of the formula (IIb),

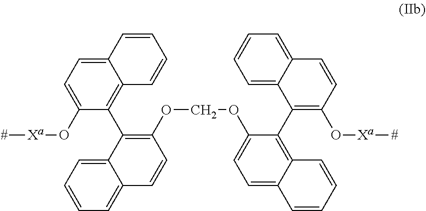

where #represents a connection point to a neighboring structural unit, where X^{a} represents the identical groups X^{1a} and X^{2a}, and where the variables X^{1a} and X^{2a} have the meanings defined herein, in particular the meanings mentioned as preferred.

Preferably, the moieties X in formula (Ib) as well as the moieties X^{a} in formula (IIb) are defined either as in one of groups (1) and (1.1) of the embodiments, in group (2) of the embodiments or in one of groups (3) and (3.1) of the embodiments. More preferably, the moieties X in formula (Ib) as well as the moieties X^{a} in formula (IIb) are defined either as in group (1.1) of the embodiments, in group (2) of the embodiments or in group (3.1) of the embodiments. In particular, the moieties X in formula (Ib) as well as the moieties X^{a} in formula (IIb) are defined as in group (1.1) of embodiments. Thus, the moieties X in formula (Ia) are here in particular selected from the group consisting of 2-hydroxyethyl (i.e. 2-(HO)-ethyl), 4-(hydroxymethyl)phenyl)methyl, (3-(hydroxymethyl)phenyl)methyl, (4-(hydroxymethyl)-1-naphthyl)methyl, (6-(hydroxymethyl)-2-naphthyl)methyl, 4′-(hydroxymethyl)-1,1′-biphenylyl-4-methyl, and especially are both 2-hydroxyethyl.

Accordingly, specific preference is given to the compound of the formula (Ib), where both moities X are 2-hydroxyethyl. Likewise, specific preference is given to the structural unit of the formula (lib), where both moieties X^{a} are 2(-O)-ethyl. In other words, among the compounds of formula (Ib) particular preference is given to the compound 2,2′-[methylenebis(oxy[1,1′-binaphthalene]-2′,2-diyloxy)]di(ethan-1-ol).

The compounds of the formula (Ia), where X is hydrogen and A¹ is a mono- or polycyclic (het)arylene moiety as defined herein, are preferably prepared by analogy to the process shown in the following reaction scheme 1, where Ar represents said mono- or polycyclic (het)arylene moiety.

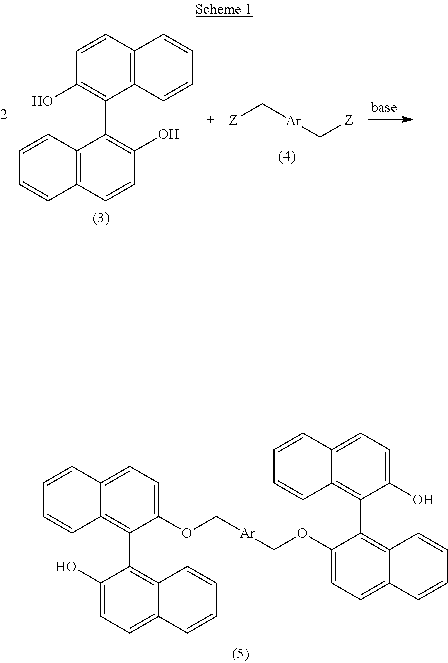

1,1′-Bi-2-naphthol of formula (3) is reacted with the a compound of formula (4), where Z is a suitable leaving group, such as a chloride, bromide, iodide, tosylate or mesitylate group, in the presence of a base, e.g. an oxo base, such as an alkaline carbonate like potassium carbonate, to yield the compound (5), which is a compound of the formula (Ia), where A¹ is a mono- or polycyclic (het)arylene moiety Ar.

The compounds of the formula (Ia), where X is hydrogen and A¹ is a moiety —CH(CH₂Ar^{A})- or —C(CH₂Ar^{A})₂- herein, can, for example, be prepared by analogy to the process shown in the following reaction scheme 2.

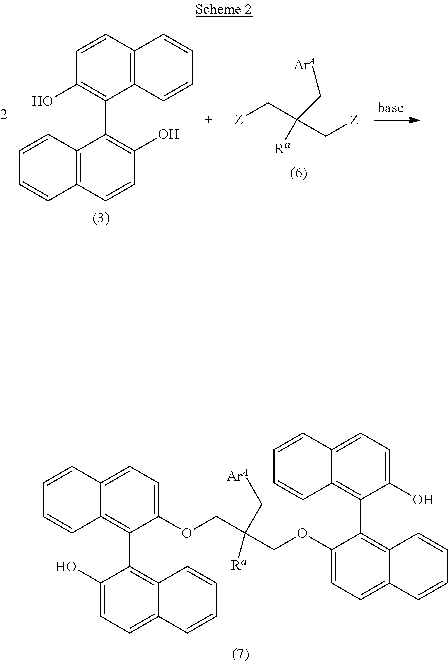

1,1′-Bi-2-naphthol of formula (3) is reacted with the a compound of formula (6), where Z is a suitable leaving group, such as a chloride, bromide, iodide, tosylate or mesitylate group, and where R^{a} is hydrogen or a group —CH₂Ar^{A}, in the presence of a base, e.g. an oxo base, such as an alkaline carbonate like potassium carbonate, to yield the compound of formula (7). The obtained compound (7) is a compound of the formula (Ia), where A¹ a moiety —CH(CH₂Ar^{A})- or —C(CH₂Ar^{A})₂- and X is hydrogen. Suitable solvents for this reaction are polar aprotic organic solvents, such as for example acetone.

The compounds of the formula (Ia), where X is hydrogen and A¹ is a single bond or —CH₂—, and the compounds of the formula (I), where m, p, q and r are all 0, n is 1, X¹ and X² are both hydrogen and the moiety —Y¹-A¹-Y²— is —CH₂—, i.e. the compounds of the formula (Ib), where X is hydrogen, can, for example, be prepared by analogy to the process shown in the following reaction scheme 3.

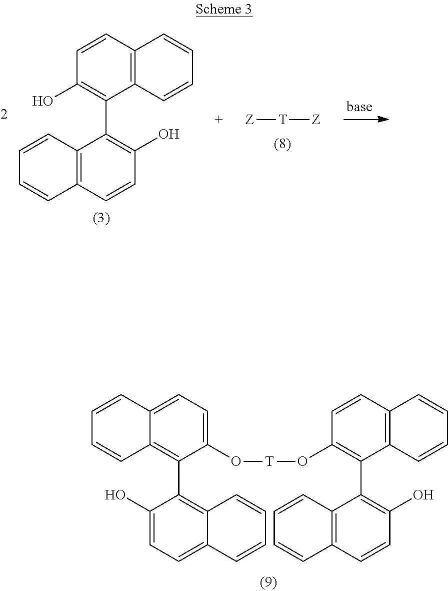

1,1′-Bi-2-naphthol of formula (3) is reacted with the a compound of formula (8), where Z is a suitable leaving group, such as a bromide, iodide, tosylate or mesitylate group, especially tosylate, and where T is a moiety —CH₂—, —CH₂CH₂— or —CH₂CH₂CH₂—, in the presence of a base, e.g. an oxo base, such as an alkaline carbonate or an alkaline hydride, especially an alkaline hydride, such as sodium hydride, to yield the compound of formula (9). The obtained compound (9) is a compound of the formula (Ia) or (Ib), where the groups X are hydrogen and A¹ in (Ia) is a single bond or a moiety —CH₂—. Suitable solvents for this reaction are polar aprotic organic solvents, such as for example dimethylformamide or else e.g. acetone if an alkali carbonate is used as the base.

The compounds of formulae (Ia) or (Ib), where X is -Alk¹-OH, —CH₂-A²-CH₂—OH, -Alk²-C(O)OR^{x} or —CH₂-A²-C(O)OR^{x} and the moiety A¹ in formula (Ia) is a single bond, Ar, —CH(CH₂Ar^{A})-, —C(CH₂Ar^{A})₂- or —CH₂—, where Ar is as defined in the context of Scheme 1 above, can, for example, be prepared from the compounds of formulae (5), (7) or (9) described above by analogy to the process shown in the following reaction scheme 4.

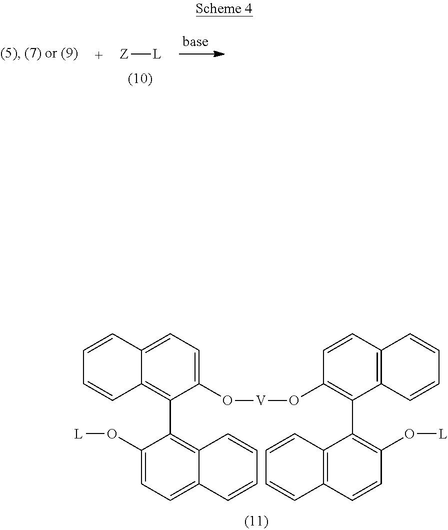

A compound of formulae (5), (7) or (9) is reacted with the a compound of formula (10), where Z is a suitable leaving group, such as a chloride, bromide, iodide, tosylate or mesitylate group and L is -Alk¹-OH, —CH₂-A²-CH₂—OH, -Alk²-C(O)OR^{x} or —CH₂-A²-C(O)OR^{x}, in the presence of a base, e.g. an oxo base, such as an alkaline carbonate to yield the compound (11), wherein V is a moiety —CH₂—Ar-CH₂, —CH₂—CH(CH₂Ar^{A})-CH₂—, —CH₂—C(CH₂Ar^{A})₂-CH₂—, —CH₂—, —CH₂CH₂— or —CH₂CH₂CH₂—, where Ar is as defined in the context of Scheme 1 above. Thus, if V is —CH₂— the compound (11) is a compound (Ib) of the invention, where X has one of the meanings listed above for the group L, or is a compound (Ia), where the unit —Y¹-A¹-Y²— has one of the meanings given above for the unit V that are different from —CH₂— and where the group X has one of the meanings listed above for the group L. Suitable solvents for this reaction of scheme 4 are polar aprotic organic solvents, such as for example dimethylformamide.

In case the group L in the compound of formula (11) is hydroxylethyl, the conversion shown in scheme 4 above is preferably conducted using 2-chloro-ethanol, or alternatively, ethylene carbonate or ethylene oxide, in particular ethylene carbonate, instead of the compound of formula (10). Such conversions with 2-chloro-ethanol, ethylene carbonate or ethylene oxide are carried out in the presence of a base, e.g. an oxo base, such as an alkaline carbonate like potassium carbonate.

As a preferred alternative to the conversions shown in schemes 2 to 4 above, the compounds of the formulae (Ia) or (Ib), where X is hydrogen, -Alk¹-OH, —CH₂-A²-CH₂—OH, -Alk²-C(O)OR^{x} or —CH₂-A²-C(O)OR^{x}, especially hydrogen, -Alk¹-OH or -Alk²-C(O)OR^{x} and the moiety A¹ in formula (Ia) is a single bond, —CH(CH₂Ar^{A})-, —C(CH₂Ar^{A})₂- or —CH₂—, can for example also be prepared by analogy to the 3- to 4-step process shown in the following reaction scheme 5.

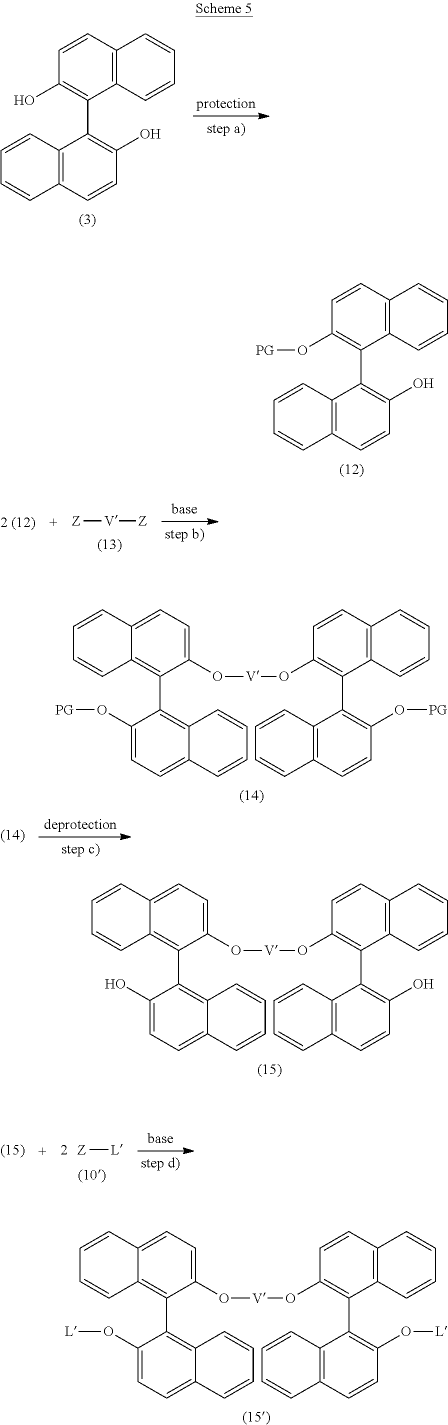

In step a) of the process only one of the two hydroxyl groups of 1,1′-bi-2-naphthol of formula (3) is protected by introducing a protecting group, which is preferably an arylmethyl group, such as in particular a benzyl group. Arylmethyl groups, particularly a benzyl group can be introduced by procedures well established in the art, e.g. by reacting 1,1′-bi-2-naphthol (3) with usually 0.5 to 1.5 molar equivalents of benzyl bromide or benzyl chloride in the presence of a suitable base, such as for instance an alkaline metal carbonate, e.g. potassium carbonate, or an alkaline metal hydride, e.g. sodium hydride. The thus obtained mono-protected derivative of formula (12), where PG is a suitable protecting group, such as a benzyl group. In the following step b) the monoprotected 1,1′-bi-2-naphthol derivative (12) is reacted with a compound of formula (13), where Z is a suitable leaving group, such as a bromide, iodide, tosylate or mesitylate group, and where V′ is a moiety —CH₂—CH(CH₂Ar^{A})-CH₂—, —CH₂—C(CH₂Ar^{A})₂-CH₂—, —CH₂—, —CH₂CH₂— or —CH₂CH₂CH₂—, in the presence of a base, for instance an oxo base, such as an alkaline metal carbonate, e.g. potassium carbonate, or an alkaline metal hydride, e.g. sodium hydride, to yield the compound of formula (14). In the subsequent step c), deprotection of the compound (14) to the corresponding diol of formula (15) can be carried out using well-established measures, such as e.g. catalytic hydrogenation if the protecting group to be removed is benzyl. The obtained diol (15) is a compound of the formulae (Ia) or (Ib), where X is hydrogen, and the moiety A¹ in formula (Ia) is a single bond, —CH(CH₂Ar^{A})-, —C(CH₂Ar^{A})₂- or —CH₂—. In optional step d) the diol (15) is converted to a corresponding compound of formula (15′), which is also a compound of formulae (la) or (Ib), where, however, X, instead of hydrogen, is Alk¹-OH, —CH₂-A²-CH₂—OH, -Alk²-C(O)OR^{x} or —CH₂-A²-C(O)OR^{x}, and particularly is -Alk¹-OH or -Alk²-C(O)OR^{x} or —CH₂-A²-C(O)OR^{x}. Apparently, the conversion of a diol (15) into compound (15′) can be accomplished for example in analogy of the procedures described in connection with scheme 4 above, by reacting the diol (15) with a compound of formula (10′), where Z has the same meanings as described for the compound (10), and L′ is -Alk¹-OH, —CH₂-A²-CH₂—OH, -Alk²-C(O)OR^{x} or —CH₂-A²-C(O)OR^{x}, especially -Alk¹-OH or -Alk²-C(O)OR^{x}. This reaction sequence is particularly useful for generating compounds of formulae (Ia) and (Ib), where X is hydrogen, -Alk¹-OH or -Alk²-C(O)OR^{x} and the moiety A¹ in formula (Ia) is a single bond, —CH(CH₂Ar^{A})-, —C(CH₂Ar^{A})₂- or —CH₂—.

As a further preferred alternative to the conversions shown in schemes 2 to 4 above, the compounds of the formulae (Ia) or (Ib), wherein X is —CH₂-A²-CH₂—OH or —CH₂-A²-C(O)OR^{x} and the moiety A¹ in formula (Ia) is a single bond, Ar, —CH(CH₂Ar^{A}), —C(CH₂Ar^{A})₂- or —CH₂—, in particular a single bond, —CH(CH₂Ar^{A})-, —C(CH₂Ar^{A})₂- or —CH₂—, where Ar is as defined in the context of Scheme 1 above, can for example also be prepared by analogy to the 2-step process shown in the following reaction scheme 6.

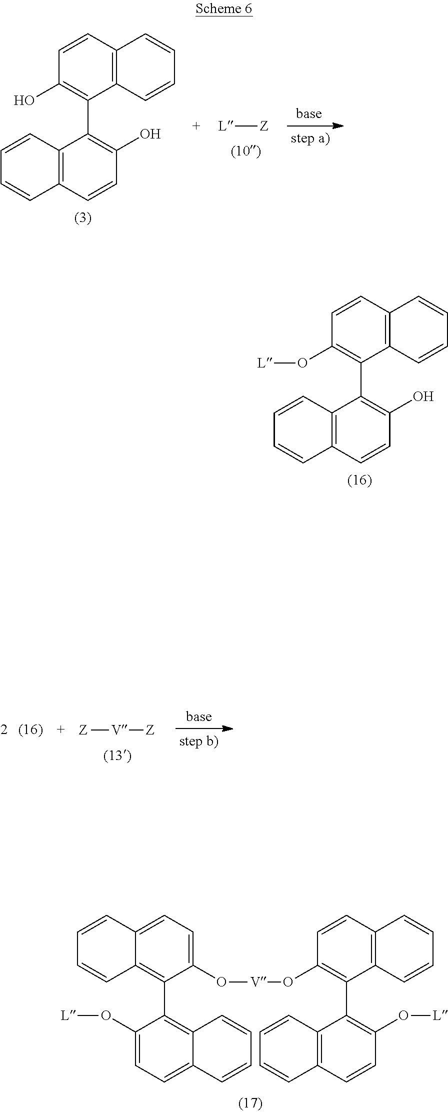

In step a) of the process 1,1′-bi-2-naphthol of formula (3) is reacted with usually 0.5 to 1.5 molar equivalents of a compound of the formula (10″), where Z is a suitable leaving group, such as a chloride, bromide, iodide, tosylate or mesitylate group and L″ is —CH₂-A²-CH₂—OH or —CH₂-A²-C(O)OR^{x}, in the presence of a base, e.g. an oxo base, such as an alkaline metal carbonate, e.g. potassium carbonate, to yield the compound (16). A suitable solvent for this reaction step is preferably selected from polar aprotic organic solvents, such as for example dimethylformamide. In step b) the compound (16) is reacted with a compound of formula (13′), where Z is a suitable leaving group, such as a chloride, bromide, iodide, tosylate or mesitylate group, and where V″ is a moiety —CH₂—Ar-CH₂—, —CH₂—CH(CH₂Ar^{A})-CH₂—, —CH₂—C(CH₂Ar^{A})₂-CH₂—, —CH₂—, —CH₂CH₂— or —CH₂CH₂CH₂—, especially a moiety —CH₂—CH(CH₂Ar^{A})-CH₂—, —CH₂—C(CH₂Ar^{A})₂-CH₂—, —CH₂—, —CH₂CH₂— or —CH₂CH₂CH₂—, in the presence of a base, e.g. an oxo base, such as an alkaline carbonate like potassium carbonate. Suitable solvents for this reaction step are polar aprotic organic solvents, such as for example acetone. The compound of formula (17) obtained by this reaction is the desired compound of formula (Ia) or (Ib) particularly characterized in that X is a moiety —CH₂-A²-CH₂—OH or —CH₂-A²-C(O)OR^{x}. This reaction sequence is particularly useful for generating compounds of formulae (Ia) and (Ib), where X is —CH₂-A²-CH₂—OH or —CH₂-A²-C(O)OR^{x}, and where in formula (Ia) the moiety A¹ is a single bond, —CH(CH₂Ar^{A})-, —C(CH₂Ar^{A})₂- or —CH₂—.

As an alternative to the conversions shown in schemes 1 to 6 above, compounds of the formulae (Ia) or (Ib), where X is different from hydrogen, can for example also be prepared by analogy to the 4-step process shown in the following reaction scheme 7.

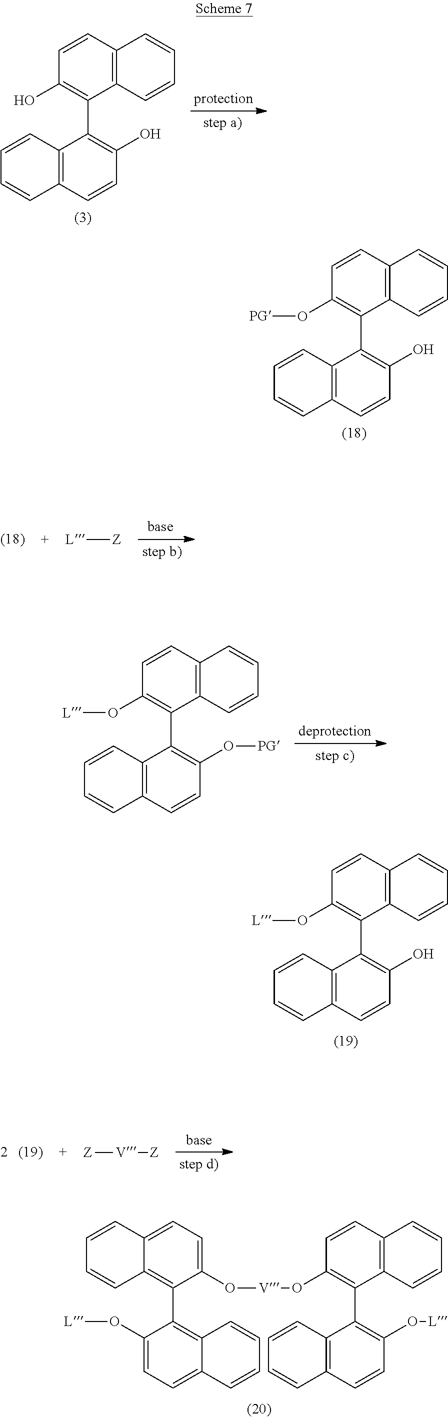

In step a) of the process 1,1′-bi-2-naphthol of formula (3) is mono-protected by introducing a suitable protecting group PG′, which is selected from hydroxyl protecting groups established in the art, such as for example 2-tetrahydropyranyl, benzyl, diphenylmethyl, trityl, allyl, propargyl or tert-butyloxycarbonyl (Boc), yielding a compound of formula (18). As apparent to a skilled person the PG′ group should be chosen to be compatible with the subsequent reactions. For example, if in step d) the variable A¹ in the compound of formula (20) is a moiety Ar, which is as defined in the context of Scheme 1 above, PG′ should be preferably be different from benzyl. The introduction of the protecting group PG′ in step a) as well as its removal in step c), which affords an alcohol of the formula (19), can be conducted in analogy to respective procedures established in the art (see e.g. EP 0 915 073 B1; T. Song et al., Advanced Synthesis & Catalysis 2014, 356(8), 1708-1718; C. Dong et al., Catalysis Science & Technology 2015, 5(10), 4755-4759; L. Jin et al., Tetrahedron: Asymmetry 2008, 19(16), 1947-1953; A. R. Abreu et al., Tetrahedron 2010, 66(3), 743-749; Y. Wang et al., Journal of the American Chemical Society 2012, 134(7), 3342-3345; H. Hocke et al., Tetrahedron 2003, 59(5), 619-630; G. Ma et al., Angewandte Chemie, International Edition 2014, 53(44), 11818-11821; M. Nuruzzaman et al., Synlett 2008, 2, 207-212; and S. C. Jha et al., Synthetic Organic Chemistry 2003, 33:6, 1005-1009). The intermediate step b) of the process for introducing a group L′″, which is -Alk¹-OH, —CH₂-A²-CH₂—OH, -Alk²-C(O)OR^{x} or —CH₂-A²-C(O)OR^{x}, can be conducted e.g. in analogy to the reaction of scheme 4 above. The final step d) yielding the desired product of formula (20), wherein the moiety V′″ is —CH₂—Ar-CH₂—, —CH₂—CH(CH₂Ar^{A})-CH₂—, —CH₂—C(CH₂Ar^{A})₂-CH₂—, —CH₂—, —CH₂CH₂— or —CH₂CH₂CH₂—, can for example be carried out in analogy to any one of the reactions of schemes 1 to 3 and of steps b) in schemes 5 and 6.

The above reactions according to schemes 1 to 7, especially schemes 1 to 4, may also applied for preparing compounds of formula (I), where the variable n is different from 1, i.e. is 2 or 3, or for preparing a mixture of compounds of formula (I) which differ only by their values of the variable n. As apparent to a skilled person, by adequately adapting the reaction conditions of the reactions of schemes 1 to 7 described above, especially schemes 1 to 3, it is typically possible to shift these reactions towards such compounds of formula (I) with n being 2 or 3. This can be accomplished, for example, by suitably increasing the quantitative ratios of the compound of formulae (4), (6) and (8), respectively, to 1,1′-bi-2-naphthol (3) used for the reactions of schemes 1 to 3.

Compounds of the formula (I), where the naphthyl moieties bear substituents R¹, R², R³ and/or R⁴, can be prepared by analogy to the reactions of schemes 1 to 7 described above, at least if R¹ and R² are identical with R³ and R⁴, respectively. For this purpose the 1,1′-bi-2-naphthol (3) used in the reactions of schemes 1 to 7 are replaced by a 1,1′-bi-2-naphthol derivative that is substituted correspondingly with R¹ (=R³) and optionally also with R² (=R⁴).

The conversions shown in schemes 1 to 7 can be accomplished by the reactions described above in the context with these schemes or by apparent variations of these reactions, or, alternatively, by procedures well-established in preparative organic chemistry, or combinations thereof.

Further compounds of formula (I) can be prepared by employing apparent variations of the reactions described above and combinations thereof with procedures well-established in preparative organic chemistry.

The reaction mixtures obtained in the individual steps of the syntheses for preparing the compounds described in reaction schemes 1, 2, 3, 4, 5, 6 and 7 above are usually worked up in a conventional way, e.g. by mixing with water, separating the phases and, where appropriate, purifying the crude products by washing, chromatography or crystallization. The intermediates in some cases result in the form of colourless or pale brownish, viscous oils, which are freed of volatiles or purified under reduced pressure and at moderately elevated temperature. If the intermediates are obtained as solids, the purification can be achieved by recrystallization or washing procedures, such as slurry washing.

The starting compounds used in the syntheses shown in schemes 1, 2, 3, 4, 5, 6 and 7 above to prepare compounds of formula (I) are commercially available or can be prepared by methods known from the art.

As stated above, the compounds of the present invention can be obtained in high purity, which means that a product is obtained, which does not contain significant amounts of organic impurities different from the compound of formula (I), except for volatiles. Usually, the purity of compounds of formula (I) is at least 95%, in particular at least 98% and especially at least 99%, based on the non-volatile organic matter, i.e. the product contains at most 5%, in particular at most 2% and especially at most 1% of non-volatile impurities different from the compound of formula (I).

The term “volatiles” refers to organic compounds, which have a boiling point of less than 200° C. at standard pressure (105 Pa). Consequently, non-volatile organic matter is understood to mean compounds having a boiling point, which exceeds 200° C. at standard pressure.

It is a particular benefit of the invention that the compounds of formula (I) and likewise their solvates, can often be obtained in crystalline form. In the crystalline form the compound of formula (I) may be present in pure form or in the form of a solvate with water or an organic solvent. Therefore, a particular aspect of the invention relates to the compounds of formula (I), which are essentially present in crystalline form. In particular, the invention relates to crystalline forms, where the compound of formula (I) is present without solvent and to the crystalline solvates of the compounds of formula (I), where the crystals contain a solvent incorporated.

It is a particular benefit of the invention that the compounds of the formula (I) and likewise their solvates, can often be easily crystallized from conventional organic solvents. This allows for an efficient purification of the compounds of formula (I). Suitable organic solvents for crystallizing the compounds of the formula (I) or their solvates, include but are not limited to aromatic hydrocarbons such as toluene or xylene, aliphatic ketones in particular ketones having from 3 to 6 carbon atoms, such as acetone, methyl ethyl ketone, methyl isopropyl ketone or diethyl ketone, aliphatic and alicyclic ethers, such as diethyl ether, dipropyl ether, methyl isobutyl ether, methyl tert-butyl ether, ethyl tert-butyl ether, dioxane or tetrahydrofuran, aliphatic-aromatic ethers, such as anisole, and aliphatic alcohols having 1 to 4 carbon atoms, such as methanol, ethanol or isopropanol, as well as mixtures thereof. It may be beneficial to subject a dissolved crude preparation of a compound of formula (I) to filtration, e.g. over cellite, prior to the crystallization step, in order to remove solid components that may be present in a crude preparation.

Furthermore, impurities, especially color forming impurities, that may be present in a crude preparation of a compound of formula (I) can be removed at any stage of the purification process, e.g. before a filtration step or a crystallization step, by standard procedures, such as treatment with an adsorbent, e.g. activated charcoal.

Alternatively, the compounds of the formula (I) and likewise their solvates, can be obtained in purified form by employing other simple and efficient methods for purifying the raw products of these compounds, such as in particular slurry washing the raw solids obtained directly after the conversion to prepare the compounds of formula (I). Slurry washing is typically conducted at ambient temperature or elevated temperatures of usually about 30 to 90° C., in particular 40 to 80° C. Suitable organic solvents here are in principle the same as those listed above as being suitable for crystallizing the compounds of formula (I), such as in particular the mentioned aromatic hydrocarbons, aliphatic ketones and aliphatic ethers, e.g. toluene, methyl ethyl ketone and methyl tertbutyl ether.

Accordingly, the compounds of formula (I) used for the preparation of the thermoplastic polymers, in particular the polycarbonates, as defined herein, can be easily prepared and obtained in high yield and high purity. In particular, compounds of formula (I) can be obtained in crystalline form, which allows for an efficient purification to the degree required in the preparation of optical resins. In particular, these compounds can be obtained in a purity which provides for high refractive indices and also low haze, which is particularly important for the use in the preparation of optical resins of which the optical devise is made of. In conclusion, the compounds of formula (I) are particularly useful as monomers in the preparation of the optical resins.

A skilled person will readily appreciate that the formula (I) of the monomer used corresponds to the formula (II) of the structural unit comprised in the thermoplastic resin. Likewise, the formulae (Ia) and (Ib), respectively, of the monomer used corresponds to the formulae (IIa), (IIb), respectively, of the structural unit comprised in the thermoplastic resin.

A skilled person will also appreciate that the structural units of the formulae (II), (IIa), and (IIb), are repeating units within the polymer chains of the thermoplastic resin.

In addition to the structural units of the formulae (II), (IIa) and (IIb), respectively, the thermoplastic resin may have structural units different therefrom. In a preferred embodiment, these further structural units are derived from aromatic monomers of the formula (IV) resulting in structural units of the formula (V):

HO—R^{z}-A³-R^{z}—OH (IV)

#-O—R^{z}-A³-R^{z}—O-# (V)
where
#represents a connection point to a neighboring structural unit;
A³ is a polycyclic radical bearing at least 2 benzene rings, wherein the benzene rings may be connected by W and/or directly fused to each other and/or fused by a non-benzene carbocycle and/or fused by two non-benzene carbocycles that are linked via a linker L, where A³ is unsubstituted or substituted by 1, 2 or 3 radicals R^{aa}, which are selected from the group consisting of halogen, C₁-C₆-alkyl, C₅-C₆-cycloalkyl, phenyl, naphthyl, 1,2-dihydroacenaphthylenyl, phenanthrenyl, pyrenyl, triphenylenyl, benzo[b]furanyl, dibenzo[b,d]furanyl, benzo[b]thienyl, dibenzo[b,d]thienyl and thianthrenyl;
W is selected from the group consisting of a single bond, O, C═O, S, S(O), SO₂, CH₂, CH-Ar, CAr₂, CH(CH₃), C(CH₃)₂ and a radical of the formula (A′)

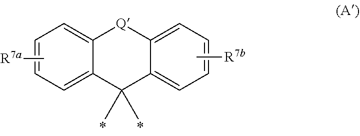

where
Q′ represents a single bond, O, C═O or CH₂;
R^{7a}, R^{7b}, independently of each other are selected from the group consisting of hydrogen, fluorine, CN, R, OR, CHᵥR′₃₋ᵥ, NR₂, C(O)R and C(O)NH₂, where R and R′ are as defined in claim 1 and v is 0, 1 or 2; and
* represents the connection point to a benzene ring;
L is selected from a single bond, C₁-C₄-alkylene, C₄-C₇-cycloalkylene, C₄-C₇-cycloalkylenedimethylene, phenylenedimethylene, where L is unsubstituted or substituted by 1 or 2 radicals R^{L}, which are selected from the group consisting of C₁-C₄-alkyl, halogen, C₁-C₄-haloalkyl, C₄-C₇-cycloalkyl and phenyl,
Ar is selected from the group consisting of mono- or polycyclic aryl having from 6 to 26 carbon atoms as ring atoms and mono- or polycyclic hetaryl having a total of 5 to 26 atoms, which are ring members, where 1, 2, 3 or 4 of these ring member atoms of hetaryl are selected from nitrogen, sulphur and oxygen, while the remainder of these ring member atoms of hetaryl are carbon atoms, where Ar is unsubstituted or substituted by 1, 2 or 3 radicals R^{ab}, which are selected from the group consisting of halogen, phenyl and C₁-C₄-alkyl;
R^{z} is a single bond, Alk³, O-Alk⁴-, O-Alk⁴-[O-Alk⁴-]_{w}- or O-Alk⁵-C(O)— where O is bound to A³, and where
   w is an integer from 1 to 10;
   Alk³ is C₁-C₄-alkandiyl;
   Alk⁴ is C₂-C₄-alkandiyl; and
   Alk⁵ is C₁-C₄-alkandiyl.

If R^{z} in formula (IV) is O-Alk⁵-C(O), the esters, in particular the C₁-C₄-alkyl esters, of the monomers of formula (IV) may be used instead.

In the context of formulae (IV) and (V), A³ is in particular either a polycyclic radical bearing 2 benzene or naphthaline rings, wherein the benzene rings are connected by W or fused by two non-benzene carbocycles that are linked via a linker L, where W is in particular selected from the group consisting of a single bond, S, S(O), SO₂, C(CH₃)₂, and a radical A′ and where L is a single bond or C₁-C₄-alkylene.

In the context of formulae (IV) and (V), R^{z} is in particular O-Alk⁴-, where Alk⁴ is in particular linear alkandiyl having 2 to 4 carbon atoms and especially O—CH₂CH₂.

Amongst the monomers of formula (IV) preference is given to monomers of the general formulae (IV-1) to (IV-8)

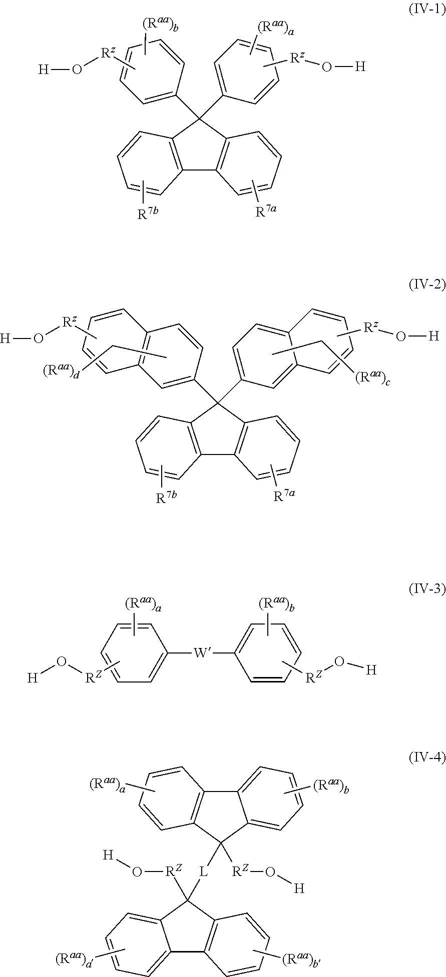

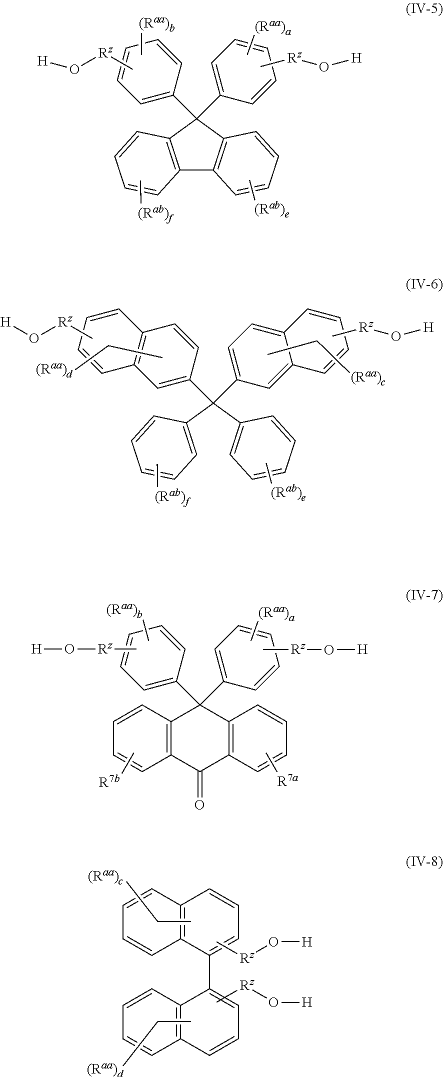

where
a and b are 0, 1, 2 or 3, in particular 0 or 1;
a′ and b′ are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1;
W′ is S, S(O), SO₂, O, single bond, CH₂, CH(CH₃), C(CH₃)₂, in particular S, S(O), SO₂ or C(CH₃)₂;
and where R^{z}, R^{aa}, R^{ab}, R^{7a}, R^{7b} and L are as defined for formula (IV) and where R^{z} is in particular selected from a single bond, CH₂ and OCH₂CH₂,

Amongst the monomers of formula (IV) particular preference is given to monomers of the general formulae (IV-11) to (IV-22), where R^{z} and R^{aa} are as defined herein and R^{z} is in particular selected from a single bond, CH₂ and O—CH₂CH₂, and especially is O—CH₂CH₂:

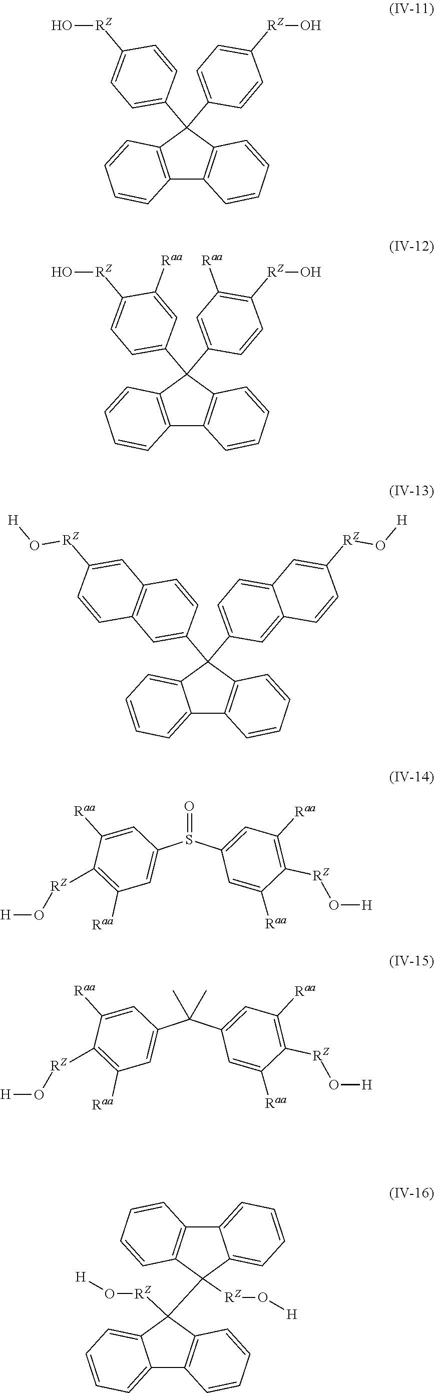

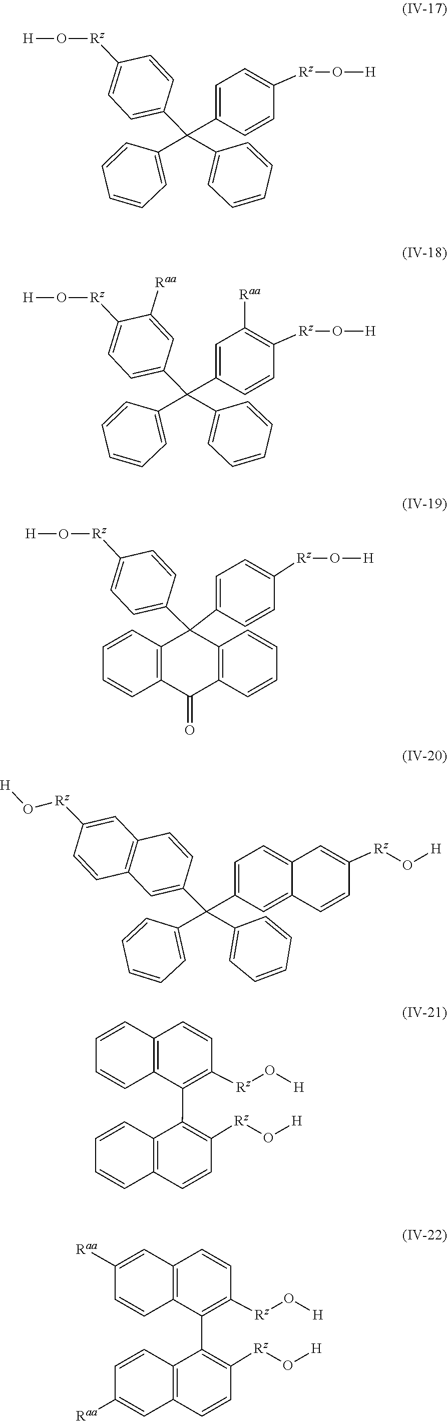

Examples of compounds of the formulae (IV-11) to (IV-22) are 9,9-bis(4-hydroxyphenyl)fluorene, 9,9-bis(4-hydroxy-3-methylphenyl)fluorene, 9,9-bis(4-hydroxy-3-isopropylphenyl)fluorene, 9,9-bis(4-hydroxy-3-tert.-butylphenyl)fluorene, 9,9-bis(4-hydroxy-3-cyclohexylphenyl)fluorene, 9,9-bis(4-hydroxy-3-phenylphenyl)fluorene, 9,9-bis(4-(2-hydroxyethoxy)phenyl)fluorene (BPEF), 9,9-bis(4-(2-hydroxyethoxy)-3-methylphenyl)fluorene, 9,9-bis(4-(2-hydroxyethoxy)-3-isopropylphenyl)fluorene, 9,9-bis(4-(2-hydroxyethoxy)-3-tert.-butylphenyl)fluorene, 9,9-bis(4-(2-hydroxyethoxy)-3-cyclohexylphenyl)fluorene, 9,9-bis(4-(2-hydroxyethoxy)-3-phenylphenyl)fluorene (BPPEF), 9,9-bis(6-hydroxy-2-naphthyl)fluorene, 9,9-bis(6-(2-hydroxyethoxy)-2-naphthyl)fluorene also termed 9,9-bis(6-(2-hydroxyethoxy)naphthalene-2-yl)fluorene (BNEF) or 6,6′-(9-fluorenylidene)bis(2-naphthyloxyethanol) (NOLE), 10,10-bis(4-hydroxyphenyl)anthracen-9-on, 10,10-bis(4-(2-hydroxyethoxy)phenyl)anthracen-9-on, 4,4′-dihydroxytetraphenylmethane, 4,4′-di-(2-hydroxyethoxy)-tetraphenylmethane, 3,3′-diphenyl-4,4′-dihydroxy-tetraphenylmethane, di-(6-hydroxy-2-naphthyl)-diphenylmethane, 2-[4-[1-[4-(2-hydroxyethoxy)-3,5-diphenyl-phenyl]-1-methyl-ethyl]-2,6-diphenyl-phenoxy]ethanol, 2-[4-[1-[4-(2-hydroxyethoxy)-3-phenyl-phenyl]-1-methyl-ethyl]-2,6-diphenyl-phenoxy]ethanol, 9,9′-dihydroxymethyl-9,9′-difluorene, 2,2′-[1,1′-binaphthalene-2,2′-diylbis(oxy)]diethanol also termed 2,2′-bis(2-hydroxyethoxy)-1,1′-binaphtyl or 2,2′-bis(2-hydroxyethoxy)-1,1′-binaphthalene (BNE), 2,2′-bis(1-hydroxymethoxy)-1,1′-binaphtyl, 2,2′-bis(3-hydroxypropyloxy)-1,1′-binaphtyl, 2,2′-bis(4-hydroxybutoxy)-1,1′-binaphtyl, 2,2′-bis(2-hydroxyethoxy)-6,6′-diphenyl-1,1′-binaphthalene, 2,2′-bis(2-hydroxyethoxy)-6,6′-di(naphthalene-1-yl)-1,1′-binaphthalene, 2,2′-bis(2-hydroxymethoxy)-6,6′-diphenyl-1,1′-binaphthalene, 2,2′-bis(2-hydroxymethoxy)-6,6′-di(naphthalene-1-yl)-1,1′-binaphthalene, 2,2′-bis(2-hydroxypropoxy)-6,6′-diphenyl-1,1′-binaphthalene, 2,2′-bis(2-hydroxypropoxy)-6,6′-di(naphthalene-1-yl)-1,1′-binaphthalene, 2,2′-bis(2-hydroxyethoxy)-6,6′-di(naphthalene-2-yl)-1,1′-binaphthalene, 2,2′-bis(2-hydroxyethoxy)-6,6′-di(9-phenanthryl)-1,1′-binaphthalene, 2-[4-[1-[4-(2-hydroxyethoxy)-3,5-di(naphthalen-1-yl)-phenyl]-1-methyl-ethyl]-2,6-di(naphthalen-1-yl)-phenoxy]ethanol, 2-[4-[1-[4-(2-hydroxyethoxy)-3,5-di(naphthalen-2-yl)-phenyl]-1-methyl-ethyl]-2,6-di(naphthalen-2-yl)-phenoxy]ethanol, 2-[4-[1-[4-(2-hydroxyethoxy)-3,5-di(phenanthren-9-yl)-phenyl]-1-methylethyl]-2,6-di(phenanthren-9-yl)-phenoxy]ethanol, 2-[4-[1-[4-(2-hydroxyethoxy)-3,5-di(1,2-dibenzo[b,d]thien-4-yl)-phenyl]-1-methyl-ethyl]-2,6-di(1,2-dibenzo[b,d]thien-4-yl)phenoxy]ethanol, 2-[4-[1-[4-(2-hydroxyethoxy)-3,5-di(thiantren-1-yl)-phenyl]-1-methylethyl]-2,6-di(thianthren-1-yl)-phenoxy]ethanol, 2-[4-[4-(2-hydroxyethoxy)-3,5-di(naphthalene-1-yl)phenyl]sulfonyl-2,6-di(naphthalene-1-yl)-phenoxy]ethanol, 2-[4-[4-(2-hydroxyethoxy)-3,5-di(naphthalene-2-yl)phenyl]sulfonyl-2,6-di(naphthalene-2-yl)-phenoxy]ethanol, 2-[4-[4-(2-hydroxyethoxy)-3,5-di(phenanthren-9-yl)phenyl]sulfonyl-2,6-di(phenanthren-9-yl)-phenoxy]ethanol, 2-[4-[4-(2-hydroxyethoxy)-3,5-di(thianthrene-1-yl)phenyl]sulfonyl-2,6-di(thianthrene-1-yl)phenoxy]ethanol and 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol and the like.

Among the monomers of the general formula (IV) or of formulae (IV-1) to (IV-8), particular preference is given to the monomers of formulae (IV-1), (IV-2), (IV-3) and (IV-8), even more preference is given to the monomers of formulae (IV-11), (IV-12), (IV-13), (IV-14), (IV-15), (IV-21) and (IV-22), and special preference given to 2,2′-bis(2-hydroxyethoxy)-1,1′-binaphtyl (BNE or BHBNA), 2,2′-bis(2-hydroxyethoxy)-6,6′-diphenyl-1,1′-binaphtyl (DPBHBNA), 9,9-bis(4-(2-hydroxyethoxy)phenyl)fluorene (BPEF), 9,9-bis(6-(2-hydroxyethoxy)-2-naphthyl)fluorene (BNEF), 9,9-bis(4-(2-hydroxyethoxy)-3-phenylphenyl)fluorene (BPPEF), 2-[4-[1-[4-(2-hydroxyethoxy)-3,5-di(phenanthren-9-yl)phenyl]-1-methylethyl]-2,6-di(phenanthren-9-yl)-phenoxy]ethanol, 2-[4-[1-[4-(2-hydroxyethoxy)-3,5-di(1,2-dibenzo[b,d]thien-4-yl)-phenyl]-1-methyl-ethyl]-2,6-di(1,2-dibenzo[b,d]thien-4-yl)-phenoxy]ethanol, 2-[4-[1-[4-(2-hydroxyethoxy)-3,5-di(thiantren-1-yl)-phenyl]-1-methyl-ethyl]-2,6-di(thianthren-1-yl)-phenoxy]ethanol, 2-[4-[4-(2-hydroxyethoxy)-3,5-di(phenanthren-9-yl)phenyl]sulfonyl-2,6-di(phenanthren-9-yl)-phenoxy]ethanol, 2-[4-[4-(2-hydroxyethoxy)-3,5-di(thianthrene-1-yl)phenyl]sulfonyl-2,6-di(thianthrene-1-yl)phenoxy]ethanol and 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol.

Accordingly, amongst the structural units of formula (V) that may be comprised in the thermoplastic resin preference is given to structural units of the general formulae (V-1) to (V-8),

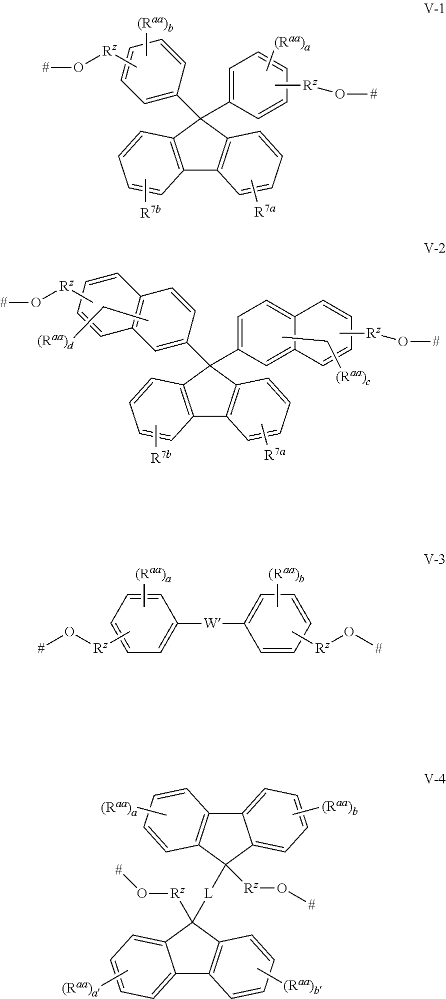

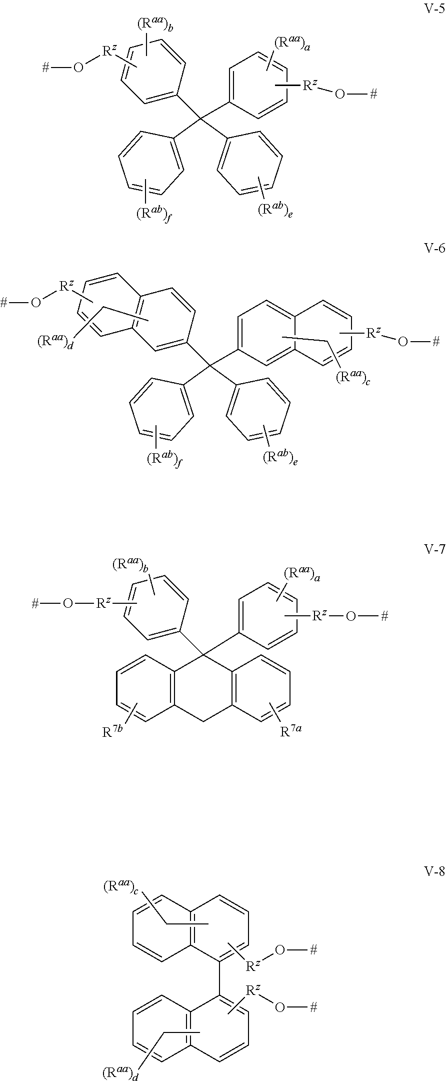

where
a and b are 0, 1, 2 or 3, in particular 0 or 1;
a′ and b′ are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1;
W′ is S, S(O), SO₂, O, single bond, CH₂, CH(CH₃), C(CH₃)₂, in particular S, S(O), SO₂ or C(CH₃)₂;
and where R^{z}, R^{aa}, R^{ab}, R^{7a}, R^{7b} and L are as defined for formula (V) and where R^{z} is in particular selected from a single bond, CH₂ and OCH₂CH₂.

Particular preference is given to structural units of the general formulae (V-11) to (V22), where R^{z} and R^{aa} are as defined herein and where R^{z} is in particular selected from a single bond, CH₂ and O—CH₂CH₂, and especially is O—CH₂CH₂:

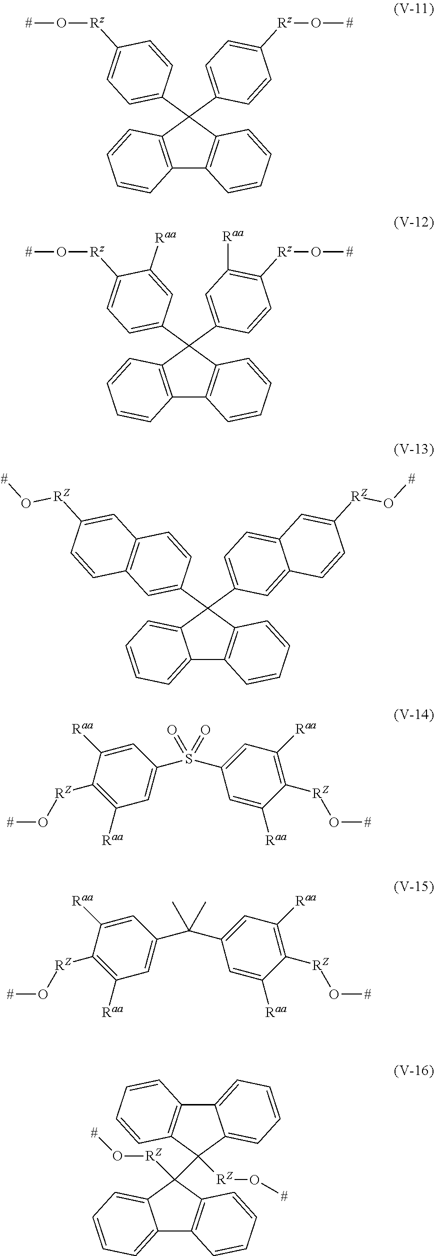

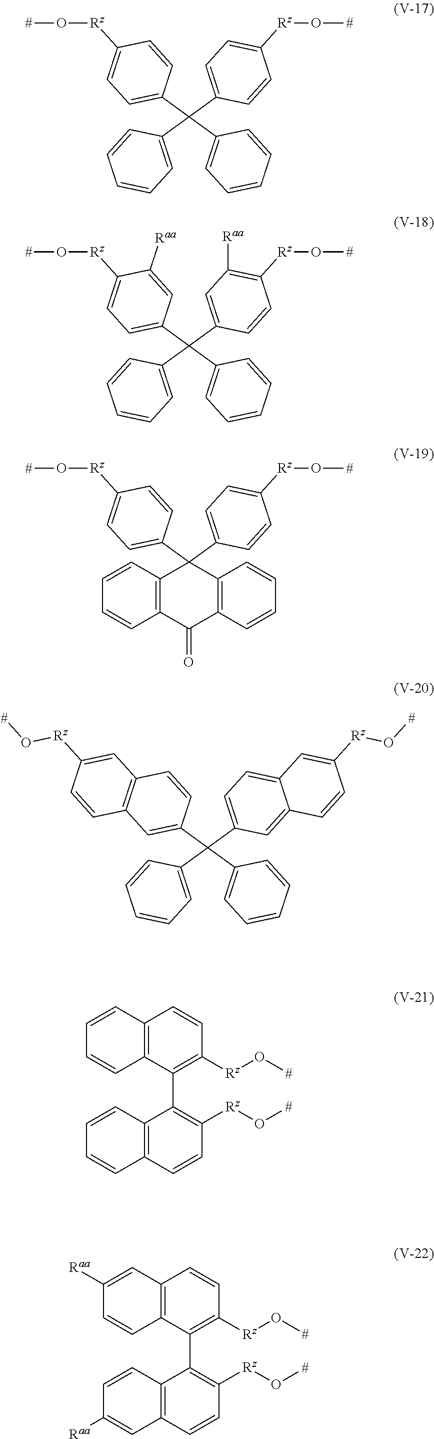

Among the structural units of the formulae (V-1) to (V-8), particular preference is given to the structural units of formulae (V-1), (V-2), (V-3) and (V-8). Among the structural units of the formulae (V-11) to (V-22), particular preference is given to the structural units of formulae (V-11), (V-12), (V-13), (V-14), (V-15), (V-21) and (V-22), and special preference given to structural units derived from 2,2′-bis(2-hydroxyethoxy)-1,1′-binaphtyl (BNE or BHBNA), 2,2′-bis(2-hydroxyethoxy)-6,6′-diphenyl-1,1′-binaphtyl (DPBHBNA), 9,9-bis(4-(2-hydroxyethoxy)phenyl)fluorene (BPEF), 9,9-bis(6-(2-hydroxyethoxy)naphthalene-2-yl)fluorene (BNEF), 9,9-bis(4-(2-hydroxyethoxy)-3-phenylphenyl)fluorene (BPPEF), 2-[4-[4-(2-hydroxyethoxy)-3,5-di(thianthrene-1-yl)phenyl]sulfonyl-2,6-di(thianthrene-1-yl)phenoxy]ethanol, 2-[4-[4-(2-hydroxyethoxy)-3,5-di(phenanthren-9-yl)phenyl]sulfonyl-2,6-di(phenanthren-9-yl)-phenoxy]ethanol, 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol, 2-[4-[1-[4-(2-hydroxyethoxy)-3,5-di(phenanthren-9-yl)-phenyl]-1-methylethyl]-2,6-di(phenanthren-9-yl)-phenoxy]ethanol, 2-[4-[1-[4-(2-hydroxyethoxy)-3,5-di(1,2-dibenzo[b,d]thien-4-yl)-phenyl]-1-methyl-ethyl]-2,6-di(1,2-dibenzo[b,d]thien-4-yl)-phenoxy]ethanol and 2-[4-[1-[4-(2-hydroxyethoxy)-3,5-di(thiantren-1-yl)-phenyl]-1-methyl-ethyl]-2,6-di(thianthren-1-yl)-phenoxy]ethanol.

In a particular preferred group of embodiments, the thermoplastic resin of the present invention comprises at least one structural unit of the formulae (IIa) or (IIb) and at least one structural unit selected from the group consisting of structural units of the formula (V-11), structural units of the formula (V-12), structural units of the formula (V-13), structural units of the formula (V-14), structural units of the formula (V-15), structural units of the formula (V-21) and structural units of the formula (V-22). In this particular group of embodiments, those thermoplastic resins are preferred, where in the structural units of the formulae (V-11), (V-12), (V-13), (V-14), (V-15), (V-21) and (V-22) the radicals R^{z} are O—CH₂CH₂.

In the thermoplastic resins of this particular preferred group of embodiments, it is preferred that the total molar ratio of the structural units of the formulae (IIa) or (IIb) is in the range from 1 to 99 mol-%, preferably in the range from 5 to 98 mol-%, further preferably in the range from 10 to 97 mol-%, and even further preferably in the range from 20 to 95 mol-% of the total amount of structural units of the formulae (II) and (V).

The compounds of the formulae (IV), (IV-1), (IV-2), (IV-3), (IV-4), (IV-5), (IV-6), (IV-7), (IV-8), (IV-11), (IV-12), (IV-13), (IV-14), (IV-15), (IV-16), (IV-17), (IV-18), (IV-19), (IV-20), (IV-21) and (IV-22) are known or can be prepared by analogy to known methods.

For example, the compounds of the formula (IV-8) can be prepared by various synthesis methods, as disclosed e.g. in JP Publication No. 2014-227387, JP Publication No. 2014-227388, JP Publication No. 2015-168658, and JP Publication No. 2015-187098. For example, 1,1′-binaphthols may be reacted with ethylene glycol monotosylates; alternatively, 1,1′-binaphthols may be reacted with alkylene oxides, halogenoalkanols, or alkylene carbonates; and alternatively, 1,1′-binaphthols may be reacted with ethylene carbonates. Thereby, the compounds of the formula (IV-8) are obtained, where R^{z}—OH is O-Alk²- or O-Alk²-[O-Alk²-]ₚ-.

For example, the compounds of the formula (IV-2) can be prepared by various synthesis methods, as disclosed e.g. in JP Patent Publication No. 5442800, and JP Publication No. 2014-028806. Examples include:
(a) reacting fluorenes with hydroxy naphthalenes in the presence of hydrochloride gas and mercapto-carboxylic acid;
(b) reacting 9-fluorene with hydroxy naphthalenes in the presence of acid catalyst (and alkyl mercaptan);
(c) reacting fluorenes with hydroxy naphthalenes in the presence of hydrochloride and thiols (such as, mercapto-carboxylic acid);
(d) reacting fluorenes with hydroxy naphthalenes in the presence of sulfuric acid and thiols (such as, mercapto-carboxylic acid) and thereafter to crystallize the product from a crystallization solvent which consists of hydrocarbons and a polar solvent(s) to form bisnaphthol fluorene; and the like.

Thereby, compounds of the formula (IV-2) can be obtained, where R^{z} is a single bond.

The compounds of formulae (IV), where R^{z} is O-Alk²- or O-Alk²-[O-Alk²-]ₚ- can be prepared from compounds of formulae (IV), where R^{z} is a single bond, by reaction with alkylene oxides or haloalkanols. For example, reacting 9,9-bis(hydroxynaphthyl)-fluorenes of the formula (IV-2) where R^{z} is a single bond with alkylene oxides or haloalkanols results in the compounds of the formula (IV-2) where R^{z} is O-Alk²- or O-Alk²-[O-Alk²-]ₚ-. For example, 9,9-bis[6-(2-hydroxyethoxy)naphthyl]fluorene can be prepared by reacting 9,9-bis[6-(2-hydroxynaphthyl]fluorene with 2-chloroethanol under alkaline conditions.

The monomers of formula (I) and likewise the co-monomers of formula (IV) used for producing the thermoplastic resin may contain certain impurities resulting from their preparation, e.g. hydroxy compounds, which bear an OH group instead of e.g. a group O-Alk¹-OH, or may contain a group O-Alk¹-[O-Alk¹]ₒ- instead of a group O-Alk¹-, or, if just one oligomeric monomer with n=1, 2, or 3, instead of a mixture of oligomers differing in their values of n, is intended, may also contain unwanted oligomers in addition to the intended oligomer. The total amount of such impurity compounds is preferably 5000 ppm or lower, more preferably 3000 ppm or lower, still more preferably 2000 ppm or lower, and especially preferably 1000 ppm or lower. The total content of the impurities in the monomers used for preparing the thermoplastic resin is preferably 4000 ppm or lower in particular 1500 ppm or lower, and more preferably 1000 ppm or lower. In particular, the total amount of dihydroxy compounds in which a carbon number of at least one of the radicals X¹ or X² differs from the formula (I), is preferably 3000 ppm or lower, more preferably 1500 ppm or lower, still more preferably 1000 ppm or lower, and especially preferably 500 ppm or lower; in the monomer(s) of which the main component is the dihydroxy compound(s) represented by the formula (I). The total content of the dihydroxy compounds in which a carbon number of at least one of the radicals X¹ or X² differs from the formula (I) is further preferably 1000 ppm or lower, and more preferably 500 ppm or lower. Likewise, the amount of impurities in the co-monomers of formula (IV) will be in the range given for the monomers of formula (I).

In this context, it should again be noted that mixtures of compounds of formula (I), which differ in their values for the variable n, are also part of the present invention.

Such mixtures, just like the individual compounds of formula (I), are well suited as monomers for the production of thermoplastic resins with advantageous properties. Thus, for producing a thermoplastic resin of the present invention a monomer of formula (I) with n=1, a monomer of formula (I) with n=2, a monomer of formula (I) with n=3, or any mixture of these compounds may be used.

Suitable thermoplastic resins for the preparation of optical devices, such as lenses, are in particular polycarbonates, polyestercarbonates and polyesters. Preferred thermoplastic resins for the preparation of optical devices, such as lenses, are in particular polycarbonates.

Said polycarbonates are structurally characterized by having structural units of at least one of the formulae (II), (IIa) and (IIb), respectively, optionally structural units derived from diol monomers, which are different from the monomer compound of the formula (I), e.g. structural units of the formula (V),

#-O—R^{z}-A³-R^{z}—O-# (V)
where
#, R^{z} and A³ are as defined herein above;
   and a structural unit of formula (III-1) stemming from the carbonate forming component:

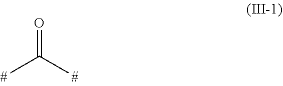

where each #represents a connection point to a neighboring structural unit, i.e. to 0 at the connection point of the structural unit of the formula (II) and, if present, to 0 at the connection point of the structural unit of the formula (V).

Said polyesters are structurally characterized by having structural units of at least one of the formulae (II), (IIa) and (IIb), respectively, optionally structural units derived from diol monomers which are different from the monomer compound of the formula (I), e.g. structural units of the formula V. If X^{1a} and X^{2a} in formulae (II), (IIa) and (IIb) are selected from a single bond, -Alk¹-O— and —CH₂-A²-CH₂—O—, the polyesters may have structural units derived from one or more dicarboxylic acids, e.g. of formula (III-2) in case of a benzene dicarboxylic acid, of formula (III-3) in case of a naphthalene carboxylic acid, of formula (III-4) in case of oxalic acid and of formula (III-5) in case of malonic acid:

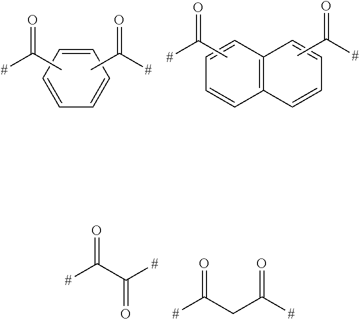

In formula (III-2) to (III-5) each variable #represents a connection point to a neighboring structural unit, i.e. to O of the connection point of the structural unit of the formula (II) and, if present, to O of the connection point of the structural unit of the formula (V).

Said polyestercarbonates are structurally characterized by having structural units of at least one of the formulae (II), (IIa) and (IIb), respectively, optionally structural units derived from diol monomers which are different from the monomer compound of the formula (I), e.g. structural units of the formula (V), a structural unit of formula (III-1) stemming from the carbonate forming component and structural units derived from dicarboxylic acid, e.g. of formula (III-2) in case of a benzene dicarboxylic acid, of formula (III-3) in case of a naphthalene carboxylic acid, of formula (III-4) in case of oxalic acid and of formula (III-5) in case of malonic acid.

A particular group of embodiments relates to thermoplastic copolymer resins, in particular polycarbonates, polyestercarbonates and polyesters, which have both structural units of formula (II) and one or more structural units of formula (V), i.e. resins, in particular polycarbonates, polyestercarbonates and polyesters, which are obtainable by reacting at least one monomer of formula (I) with one or more monomers of formula (IV). In this case the molar ratio of monomers of formula (I) to monomers of formula (IV) and likewise the molar ratio of the structural units of formula (II) to structural units of formula (V) are in the range from 1:99 to 99:1, in particular in the range from 20:80 to 98:2 and especially in the range from 30:70 to 97:3 or in the range from 10:90 to 99:1, in particular in the range from 15:85 to 98:2, 15:85 to 90:10, 15:85 to 80:20 or 15:85 to 70:30, more preferably in the range from 20:80 to 97:3 or 20:80 to 85:15, or in the range from 25:75 to 97:3, 25:75 to 85:15 or 25:75 to 80:20, especially in the range from 27:73 to 75:25, 27:73 to 80:20, 27:73 to 97:3 or in the range from 27:73 to 99:1, even more preferably in the range from 27:73 to 90:10 and specifically in the range from 30:70 to 80:20 or in the range from 35:65 to 70:30. Accordingly, the molar ratio of the structural units of the formula (11) is usually from 1 to 99 mol-% in particular from 20 to 98 mol-%, more preferably in the range from 30 to 97 mol-% or in the range from 10 to 99 mol-%, especially in the range from 20 to 97 mol-% or in the range from 27 to 97 mol-%, even more preferably in the range from 27 to 90 mol-% and specifically in range from 30 to 80 mol-% or in the range from 35 to 70 mol-%, based on the total molar amount of structural units of the formulae (11) and (V). Accordingly, the molar ratio of the structural units of the formula (V) is usually from 1 to 99 mol-%, in particular from 2 to 80 mol-%, more preferably in the range from 3 to 70 mol-% or in the range from 1 to 90 mol-%, especially in the range from 2 to 85 mol-% or in the range from 3 to 80 mol-%, even more preferably in the range of 10 to 73 mol-% and specifically in range from 20 to 70 mol-% or in the range from 30 to 65 mol-%, based on the total molar amount of structural units of the formulae (II) and (V). The above-mentioned molar ratios can be applied to the molar ratio of the structural units of formula (11) to the total structural units of the thermoplastic copolymer resins.

A specific group of embodiments relates to thermoplastic copolymer resins, in particular polycarbonates, polyestercarbonates and polyesters, which have both structural units of formula (II) and one or more structural units of formulae (V-14) or (V-15), i.e. resins, in particular polycarbonates, polyestercarbonates and polyesters, which are obtainable by reacting at least one monomer of formula (I) with one or more monomers of formulae (IV-14) or (IV-15). In this case the molar ratio of monomers of formula (I) to monomers of formulae (IV-14) and (IV-15) and likewise the molar ratio of the structural units of formula (II) to structural units of formulae (V-14) and (V-15) are in the range from 50:50 to 99:1, in particular in the range from 70:30 to 98:2 and especially in the range from 80:20 to 97:3. The above-mentioned molar ratios can be applied to the molar ratio of the structural units of formula (II) to the total structural units of the thermoplastic copolymer resins.

Another specific group of embodiments relates to thermoplastic copolymer resins, in particular polycarbonates, polyestercarbonates and polyesters, which have both structural units of formula (II) and one or more structural units of formulae (V-11), (V-12), (V13), (V-21) or (V-22), i.e. resins, in particular polycarbonates, polyestercarbonates and polyesters, which are obtainable by reacting at least one monomer of formula (I) with one or more monomers of formulae (IV-11), (IV-12), (IV-13), (IV-21) or (IV-22). In this case the molar ratio of monomers of formula (I) to monomers of formulae (IV-11), (IV-12), (IV-13), (IV-21) and (1-22) and likewise the molar ratio of the structural units of formula (II) to structural units of formulae (V-11), (V-12), (V-13), (V-21) and (V-22) are in the range from 10:90 to 90:10, 15:85 to 80:20, 20:80 to 70:30, 25:75 to 80:20, 30:70 to 90:10 or 30:70 to 80:20, in particular in the range from 35:65 to 75:25, 35:65 to 70:30, 40:60 to 85:15 or 40:60 to 80:20, and especially in the range from 50:50 to 80:20. The above-mentioned molar ratios can be applied to the molar ratio of the structural units of formula (II) to the total structural units of the thermoplastic copolymer resins.

The thermoplastic copolymer resins of the present invention, such as a polycarbonate resin may include either one of a random copolymer structure, a block copolymer structure, and an alternating copolymer structure. The thermoplastic resin according to the present invention does not need to include all of structural units (II) and one or more different structural units (V) in one, same polymer molecule. Namely, the thermoplastic copolymer resin according to the present invention may be a blend resin as long as the above-described structures are each included in any of a plurality of polymer molecules. For example, the thermoplastic resin including all of structural units (II) and structural units (V) described above may be a copolymer including all of structural units (II) and structural units (V), it may be a mixture of a homopolymer or a copolymer including at least one structural unit (II) and a homopolymer or a copolymer including at least one structural unit (V) or it may be a blend resin of a copolymer including at least one structural unit (II) and a first structural unit (V) and a copolymer including at least one structural unit (II) and at least one other structural unit (V) different from the first structural units (V); etc.

Thermoplastic polycarbonates are obtainable by polycondensation of a diol component and a carbonate forming component. Similarly, thermoplastic polyesters and polyestercarbonates are obtainable by polycondensation of a diol component and a dicarboxylic acid, or an ester forming derivative thereof, and optionally a carbonate forming component.

Specifically, thermoplastic resins (polycarbonate resins) can be prepared by the following methods.

A method for preparing the thermoplastic resin of the present invention, such as a polycarbonate resin, includes a process of melt polycondensation of a dihydroxy component corresponding to the above-mentioned structural units and a diester carbonate. According to the present invention the dihydroxy compound comprises at least one dihydroxy compound represented by the formula (I), in particular by the formulae (Ia) or (Ib), respectively, as defined herein. In addition to the compound of formula (I), the dihydroxy compound may also comprise one or more dihydroxy compounds represented by the formula (IV), preferably by the formulae (IV-1) to (IV-8), in particular by the formulae (IV-11) to (IV-22), and especially by the formulae (IV-11), (IV-12), (IV-13), (IV-14), (IV-15), (IV-21) or (IV-22).

As is clear from the above, the polycarbonate resin can be formed by reacting a dihydroxy component with a carbonate precursor, such as a diester carbonate, where the dihydroxy component comprises at least one compound represented by the formulae (I), (Ia) and (Ib), respectively, or a combination of at least one compound represented by the formulae (I), (Ia) and (Ib), respectively, and at least one compound represented by the formulae (IV), (IV-1), (IV-2), (IV-3), (IV-4), (IV-5), (IV-6), (IV-7), (IV-8), (IV-11), (IV-12), (IV-13), (IV-14), (IV-15), (IV-16), (IV-17), (IV-18), (IV-19), (IV-20), (IV-21) or (IV-22). Specifically, a polycarbonate resin can be formed by a melt polycondensation process in which the compound represented by the formulae (I), (la) and (Ib), respectively, or a combination thereof with at least one compound of the formulae (IV), (IV-1), (IV-2), (IV-3), (IV-4), (IV-5), (IV-6), (IV-7), (IV-8), (IV-11), (IV-12), (IV-13), (IV-14), (IV-15), (IV-16), (IV-17), (IV-18), (IV-19), (IV-20), (IV-21) or (IV-22) and a carbonate precursor, such as a diester carbonate, are reacted in the presence of a basic compound catalyst, a transesterification catalyst, or a mixed catalyst thereof, or in the absence of a catalyst.

A thermoplastic resin (or a polymer) other than a polycarbonate resin, such as polyestercarbonates and polyesters is obtained by using the dihydroxy compound represented by the formulae (I), (la) and (Ib), respectively, or a combination thereof with at least one compound represented by the formulae (IV), (IV-1), (IV-2), (IV-3), (IV-4-), (IV-5), (IV-6), (IV-7), (IV-8), (IV-11), (IV-12), (IV-13), (IV-14), (IV-15), (IV-16), (IV-17), (IV-18), (IV-19), (IV-20), (IV-21) or (IV-22) as a material (or a monomer).

As mentioned before, the monomers of formula (I) and likewise the co-monomers of formula (IV) used for producing the thermoplastic resin may contain impurities resulting from their preparation.

For example, the monomers of the formulae (IV-1) and (IV-2), where R^{z} is O-Alk²- or O-Alk²-[O-Alk²-]ₚ-, may include a dihydroxy compound in which both R^{z} are a single bond, or a dihydroxy compound in which one of R^{z} is a single bond, instead of O-Alk²- or O-Alk²-[O-Alk²-]ₚ-.

The total amount of such dihydroxy compounds of the formulae (IV-1) or (IV-2) in which at least one of R^{z} differs from O-Alk⁴- or O-Alk⁴-[O-Alk⁴-]_{w}-, is preferably 3000 ppm or lower, more preferably 1500 ppm or lower, still more preferably 1000 ppm or lower, and especially preferably 500 ppm or lower; in the monomer(s) of which main component is the dihydroxy compound(s) represented by the formulae (IV-1) or (IV-2). The total content of the dihydroxy compounds in which at least one of the values of a and b or c and d differs from the formula (IV-1) or (IV-2) is still preferably 300 ppm or lower, and more preferably 200 ppm or lower.

The polycarbonate resins can be obtained by reacting the monomer compounds of the formula (I) or by reacting combination of at least one monomer compound of the formula (I), in particular at least one monomer (I) mentioned herein as preferred, and one or more monomer compounds of the formula (IV), and in particular of the formulae (IV-11), (IV-12), (IV-13), (IV-14), (IV-15), IV-21) or (IV-22), and the like, as dihydroxy components; with carbonate precursors, such as diester carbonates.

However, in a polymerization process for manufacturing the polycarbonate resins, some compounds of the formulae (I) and (IV) may be converted into impurities, where one of or both of the terminal —X¹, —X² or —R^{z}OH radicals are replaced with a different radical, such as a vinyl terminal radical represented by —OCH═CH₂. Because the amount of such impurities is generally small, the products of the formed polymers can be used as polycarbonate resins without a purification process.

The thermoplastic resin of the present invention may also contain minor amount of impurities, for example, as extra contents of thermoplastic resin composition or a part of the polymer skeleton of the thermoplastic resin. The examples of such impurities include phenols formed by a process for forming the thermoplastic resin, unreacted diester carbonates and monomers. The total amount of impurities in the thermoplastic resin may be 5000 ppm or lower, or 2000 ppm or lower. The total amount of impurities in the thermoplastic resin is preferably 1000 ppm or lower, more preferably 500 ppm or lower, still more preferably 200 ppm or lower, and especially preferably 100 ppm or lower.

The total amount of phenols as impurities in the thermoplastic resin may be 3000 ppm or lower, or 2000 ppm or lower. The total amount of phenols as impurities is preferably 1000 ppm or lower, more preferably 800 ppm or lower, still more preferably 500 ppm or lower, and especially preferably 300 ppm or lower.

The total amount of diester carbonates as impurities in the thermoplastic resin is preferably 1000 ppm or lower, more preferably 500 ppm or lower, still more preferably 100 ppm or lower, and especially preferably 50 ppm or lower.

The total amount of unreacted monomers as impurities in the thermoplastic resin is preferably 3000 ppm or lower, more preferably 2000 ppm or lower, still more preferably 1000 ppm or lower, and especially preferably 500 ppm or lower.

The lower limit of the total amount of these impurities is not important, but may be 0.1 ppm, or 1.0 ppm.

The total amount of residual heavy metals, e.g. palladium, as impurity in the thermoplastic resin is preferably 50 ppm or lower, more preferably 10 ppm or lower. The amount of residual palladium can be reduced by standard procedures like treatment with an adsorbent, e.g. active charcoal.

Resins having targeted characteristics can be formed by adjusting the amounts of phenols and diester carbonates. The amounts of phenols, diester carbonates, and monomers can be suitably adjusted by arranging the conditions for polycondensation, the working conditions of devices used for polymerization, or the conditions for extrusion molding after the polycondensation process.

The weight-average molecular weight (Mw), as determined by GPC (gel permeation chromatography), of the thermoplastic resin according to the present invention is preferably in the range from 5000 to 100000 Dalton, more preferably 10000 to 80000 Dalton or 20000 to 65000 Dalton, especially in the range of 10000 to 50000 Dalton or 20000 to 40000 Dalton. The GPC measurements may be calibrated by using polystyrene standards. The Mw of a thermoplastic resin of the present invention determined this way is also denoted herein as “polystyrene conversion weight-average molecular weight”. The number-average molecular weight (Mn) of the thermoplastic resin according to the present invention is preferably in the range of 3000 to 30000, more preferably 5000 to 25000, and especially in the range of 7000 to 20000. The viscosity-average molecular weight (Mv) of the thermoplastic resin according to the present invention is preferably in the range from 8000 to 28000, more preferably 9000 to 22000, and still more preferably 10000 to 18000.

The value of the molecular weight distribution (Mw/Mn) of the thermoplastic resin according to the present invention is preferably 1.5 to 9.0, more preferably 1.8 to 7.0, and still more preferably 2.0 to 4.0.

When a thermoplastic resin has the value of the weight-average molecular weight (Mw) within the above-mentioned suitable range, a molded article made from the thermoplastic resin has high strength. In addition, such a thermoplastic resin with the suitable Mw value is advantageous for molding because of its excellent fluidity.

Preferably, the thermoplastic resin comprises 9% by weight or less, in particular 7% by weight or less and especially 5% by weight or less, e. g. 0.1 to 9% by weight, in particular 0.1 to 7% by weight and especially 0.1 to 5% by weight, of low molecular weight compounds having molecular weight of less than 1000, based on the total weight of the thermoplastic resin. If such low molecular weight compounds are present in the thermoplastic resin in an amount within the above ranges, the mechanical strength of a molded body made from such a thermoplastic resin is commonly increased, especially compared to a molded body made from a thermoplastic resin with a higher amount of the low molecular weight compounds. In addition, a thermoplastic resin according to this embodiment comprising 9% by weight or less, in particular 7% by weight or less and especially 5% by weight of low molecular weight compounds with molecular weights of less than 1000, is not or only slightly prone to precipitation of the low molecular weight compounds, which is also known as bleed-out during a molding process, such as an injection molding process. In contrast, molding of a thermoplastic resin with a higher amount of the low molecular weight compounds may be accompanied by bleed-out to a greater extent. It is also preferred that the thermoplastic resin comprises 0.1% by weight or more, in particular 0.3% by weight or more or 0.5% by weight or more, and especially 1.0% by weight or more, e. g. 0.5 to 9% by weight, in particular 1 to 9% by weight or 1 to 7% by weight and especially 1 to 5% by weight, of low molecular weight compounds with molecular weights of less than 1000, based on the total weight of the thermoplastic resin.

The thermoplastic resin of the present invention, such as especially the above-mentioned polycarbonate resin, has a high refractive index (n_{D} or n_{d}) and thus is suitable to an optical lens. The values of the refractive index as referred herein are values of a film having a thickness of 0.1 mm may be measured by use of an Abbe refractive index meter by a method of JIS-K-7142. The refractive index of the thermoplastic resin of the present invention, in particular the polycarbonate resin of the present invention, at 23° C. and at a wavelength of 589 nm is, in case the resin includes the structural unit (II), frequently 1.630 or higher, preferably 1.640 or higher, more preferably 1.650 or higher, still more preferably 1.660 or higher, in particular 1.665 or higher, 1.670 or higher, 1.675 or higher, or 1.680 or higher, and specifically 1.685 or higher. For example, the refractive index of the copolycarbonate resin including the structural unit (II) and a structural unit (V) according to the present invention is preferably 1.640 to 1.730, preferably 1.650 to 1.730, still more preferably 1.660 to 1.730.

The Abbe number (ν or νd) of the thermoplastic resin of the present invention, in particular the polycarbonate resin of the present invention, is preferably 24 or lower, or 23 or lower, more preferably 22 or lower, or 21 or lower, and still more preferably 20 or lower, or 19 or lower. The Abbe number may be calculated by use of the following equation based on the refractive index at wavelengths of 487 nm, 589 nm and 656 nm at 23° C.:

ν=(n_{D}−1)/(n_{F}−n_{c})
n_{D}: refractive index at a wavelength of 589 nm
n_{c}: refractive index at a wavelength of 656 nm
n_{F}: refractive index at a wavelength of 486 nm

The glass transition temperature (Tg) of the thermoplastic resin of the present invention, in particular the polycarbonate resin of the present invention, is, in consideration of that the polycarbonate is usable for injection molding, frequently in the range of 90 to 185° C., preferably in the range of 90 to 180° C., more preferably in the range of 100 to 170° C. or 110 to 170° C., and especially in the range of 110 to 160° C., 120 to 165° C. or 130 to 160° C. With regard to the molding fluidity and the molding heat resistance, the lower limit of Tg is preferably 130° C. and more preferably 135° C., and the upper limit of Tg is preferably 180° C. and more preferably 170° C. A glass transition temperature (Tg) in the above given ranges provides a significant range of usable temperature and avoids the risk that the melting temperature of the resin may be too high, and thus the resin may be undesirably decomposed or colored. What is more, it allows for preparing molds having have a high surface accuracy. The values given for the glass transition temperature refer to the values measured by differential scanning calorimetry (DSC) using a 10° C./minute heating program according to the protocol of JIS K7121-1987.

In the preferred group (11) of embodiments the absolute value of the orientation birefringence of the thermoplastic resin is preferably in the range of 0 to 1×10⁻², more preferable in the range of 0 to 5×10⁻³, even more preferable in the range of 0 to 2×10⁻³, in particular in the range of 0 to 1×10⁻³, and specifically in the range of 0 to 0.4×10⁻³.

An optical molded body such as an optical element produced by using a polycarbonate resin of the present invention has a total light transmittance of preferably 85% or higher, more preferably 87% or higher, and especially preferably 88% or higher. A total light transmittance of preferably 85% or higher is as good as that provided by bisphenol A type polycarbonate resin or the like.

The thermoplastic resin according to the present invention has high moisture and heat resistance. The moisture and heat resistance may be evaluated by performing a “PCT test” (pressure cooker test) on a molded body such as an optical element produced by use of the thermoplastic resin and then measuring the total light transmittance of the molded body after the PCT test. In the PCT test, first, an injection molded body having a diameter of 50 mm and a thickness of 3 mm is kept for 20 hours with PC305S III made by HIRAYAMA Corporation under the conditions of 120° C., 0.2 MPa, 100% RH for 20 hours. Then, the sample of the injection molded body is removed from the device and the total light transmittance is measured using the SE2000 type spectroscopic parallax measuring instrument made by Nippon Denshoku Industries Co., Ltd in accordance with the method of JIS-K-7361-1.

The thermoplastic resin according to the present invention has a post-PCT test total light transmittance of 60% or higher, preferably 70% or higher, more preferably 75% or higher, still more preferably 80% or higher, and especially preferably 85% or higher. As long as the total light transmittance is 60% or higher, the thermoplastic resin is considered to have a higher moisture and heat resistance than that of the conventional thermoplastic resin.

The thermoplastic resin according to the present invention has a b value, which represents the hue, of preferably 5 or lower. As the b value is smaller, the color is less yellowish, which is good as a hue.

According to the invention, the diol component, which is used in the preparation of the polycarbonates or polyesters, may additionally comprise one or more diol monomers, which are different from the monomer compound of the formula (I), such as one or more monomers of the formula (IV).

Suitable diol monomers, which are different from the monomer compound of the formula (I), are those, which are conventionally used in the preparation of polycarbonates, e.g.
aliphatic diols such as ethylene glycol, propanediol, butanediol, pentanediol and hexanediol;
alicyclic diols such as tricyclo[5.2.1.02,6]decane dimethanol, cyclohexane-1,4-dimethanol, decalin-2,6-dimethanol, norbornane dimethanol, pentacyclopentadecane dimethanol, cyclopentane-1,3-dimethanol, spiroglycol, 1,4:3,6-dianhydro-D-sorbitol, 1,4:3,6-dianhydro-D-mannitol and 1,4:3,6-dianhydro-L-iditol are also included in examples of the diol; and
aromatic diols, in particular aromatic diols of the formula (IV) such as bis(4-hydroxyphenyl)methane, 1,1-bis(4-hydroxyphenyl)ethane, bis(4-hydroxyphenyl)ether, bis(4-hydroxyphenyl)sulfoxide, bis(4-hydroxyphenyl)sulfide, bis(4-hydroxyphenyl)sulfone, bis(4-hydroxyphenyl)ketone, 2,2-bis(4-hydroxyphenyl)propane, 2,2-bis(4-hydroxy-3-t-butylphenyl)propane, 2,2-bis(4-hydroxy-3-methylphenyl)propane, 1,1-bis(4-hydroxyphenyl)cyclopentane, 1,1-bis(4-hydroxyphenyl)cyclohexane, 2,2-bis(4-hydroxyphenyl)hexafluoropropane, bis(4-hydroxyphenyl)diphenylmethane, 1,1-bis(4-hydroxyphenyl)-1-phenylethane, α,ω-bis[2-(phydroxyphenyl)ethyl]polydimethylsiloxane, α,ω-bis[3-(o-hydroxyphenyl)propyl]polydimethylsiloxane, 4,4′-[1,3-phenylenebis(1-methylethylidene)hydroxyphenyl]-1-phenylethane, 9,9-bis(4-hydroxyphenyl)fluorene, 9,9-bis[4-(2-hydroxyethoxy)-3-methylphenyl]fluorene, 9,9-bis[4-(2-hydroxyethoxy)-3-tert-butylphenyl]fluorene, 9,9-bis[4-(2-hydroxyethoxy)-3-isopropylphenyl]fluorene, 9,9-bis[4-(2-hydroxyethoxy)-3-cyclohexylphenyl]fluorene, 9,9-bis(4-hydroxy-3-phenylphenyl)fluorene, 9,9-bis(4-(2-hydroxyethyl)phenyl)fluorene, 9,9-bis(4-(2-hydroxyethyl)-3-phenylphenyl)fluorene, 9,9-bis(6-hydroxy-2-naphthyl)fluorene, 9,9-bis(6-(2-hydroxyethyl)-2-naphthyl)fluorene, 10,10-bis(4-hydroxyphenyl)anthracen-9-on, 10,10-bis(4-(2-hydroxyethyl)phenyl)anthracen-9-on, 2-[4-[4-(2-hydroxyethoxy)-3,5-di(thianthrene-1-yl)phenyl]sulfonyl-2,6-di(thianthrene-1-yl)phenoxy]ethanol, 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol, 2-[4-[1-[4-(2-hydroxyethoxy)-3,5-di(phenanthren-9-yl)-phenyl]-1-methylethyl]-2,6-di(phenanthren-9-yl)-phenoxy]ethanol and 2,2′-[1,1′-binaphthalene-2,2′-diylbis(oxy)]diethanol, also termed 2,2′-bis(2-hydroxyethoxy)-1,1′-binaphthyl or 2,2′-bis(2-hydroxyethoxy)-1,1′-binaphthalene (BNE).

Preferably, the diol component comprises at least one monomer of the formula (IV) in addition to the monomer of formula (I). In particular, the total amount of monomers of formulae (I) and (IV) contribute to the diol component by at least 90% by weight, based on the total weight of the diol component or by at least 90 mol-%, based on the total molar amount of the diol monomers of the diol component. In particular, the diol component comprises at least one monomer selected from the monomers of formulae (IV-11) to (IV-22) in addition to the monomer of formula (I). More particularly, the diol component comprises at least one monomer selected from the monomers of formulae (IV-11), (IV-12), (IV-13), (IV-14), (IV-15), (IV-21) and (IV-22) in addition to the monomer of formula (I). Especially, the diol component comprises at least one monomer selected from 2,2′-bis(2-hydroxyethoxy)-1,1′-binaphtyl, 2,2′-bis(2-hydroxyethoxy)-6,6′-diphenyl-1,1′-binaphtyl, 9,9-bis(6-(2-hydroxyethoxy)-2-naphthyl)fluorene, 9,9-bis(4-(2-hydroxyethoxy)phenyl)fluorene, 2-[4-[4-(2-hydroxyethoxy)-3,5-di(thianthrene-1-yl)phenyl]sulfonyl-2,6-di(thianthrene-1-yl)phenoxy]ethanol, 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol, 2-[4-[1-[4-(2-hydroxyethoxy)-3,5-di(phenanthren-9-yl)-phenyl]-1-methylethyl]-2,6-di(phenanthren-9-yl)-phenoxy]ethanol and 9,9-bis(4-(2-hydroxyethoxy)-3-phenylphenyl)fluorene and combinations thereof in addition to the monomer of formula (I).

Frequently, the relative amount of monomer compound of formula (I), based on the total weight of the diol component, is at least 1% by weight, preferably at least 10% or at least 30% by weight, in particular at least 15% by weight or at least 20% by weight and especially at least 25% by weight or at least 30% by weight, preferably in the range of 1 to 99% by weight or in the range of 10 to 98% by weight, in particular in the range of 20 to 98% by weight or in the range of 25 to 98% by weight or in the range of 30 to 98% by weight or in the range 30 to 97% by weight, especially in the range of 15 to 96% by weight or in the range of 20 to 95% by weight or in the range 30 to 95% by weight or in the range of 30 to 93% by weight, but may also be as high as 100% by weight.

Frequently, the relative molar amount of monomer compound of formula (I), based on the total molar amount of the diol component, is at least 1 mol-%, preferably at least 10 mol-% or at least 30 mol-%, in particular at least 15 mol-% or at least 20 mol-% and especially at least 25 mol-% or at least 30 mol-%, preferably in the range of 1 to 99 mol-% or in the range of 10 to 98 mol-% or in the range of 20 to 98 mol-% or in the range of 25 to 98 mol-%, in particular in the range of 15 to 96 mol-% or in the range of 20 to 95 mol-% or in the range of 30 to 95 mol-% or in the range of 30 to 93 mol-%, especially in the range of 20 to 90 mol-% or in the range of 25 to 90 mol-% or in the range of 30 to 90 mol-% or in the range of 32 to 90 mol-% or in the range of 35 to 90 mol-%, but may also be as high as 100 mol-%.

Consequently, the relative molar amount of monomer compound of formula (IV), based on the total molar amount of the diol component, will not exceed 99 mol-% or 90 mol-% or 70 mol-%, in particular not exceed 85 mol-% or 80 mol-% and especially not exceed 75 mol-% or 70 mol-%, and is preferably in the range of 1 to 99 mol-% or in the range of 2 to 90 mol-% or in the range of 2 to 80 mol-% or in the range of 2 to 75 mol-%, in particular in the range of 4 to 85 mol-% or in the range of 5 to 80 mol-% or in the range of 5 to 70 mol-% or in the range of 7 to 70 mol-%, especially in the range of 10 to 80 mol-% or in the range of 10 to 75 mol-% or in the range of 10 to 70 mol-% or in the range of 10 to 68 mol-% or in the range of 10 to 65 mol-%, but may also be as high as 99.9 mol-%.

Frequently, the total molar amount of monomers of formula (I) and monomers of formula (IV) is at least 80 mol-%, in particular at least 90 mol-%, especially at least 95 mol-% or up to 100 mol-%, based on the total molar amount of the diol monomers in the diol component.

Examples of further preferred aromatic dihydroxy compound, which can be used in addition to the monomers of formula (I) and optionally monomers of formula (IV) include, but are not limited to bisphenol A, bisphenol AP, bisphenol AF, bisphenol B, bisphenol BP, bisphenol C, bisphenol E, bisphenol F, bisphenol G, bisphenol M, bisphenol S, bisphenol P, bisphenol PH, bisphenol TMC, bisphenol Z and the like.

In order to adjust the molecular weight and the melt viscosity, the monomers forming the thermoplastic polymer may also include a monofunctional compound, in case of polycarbonates a monofunctional alcohol and in case of polyesters a monofunctional alcohol or a monofunctional carboxylic acid. Suitable monoalcohols are butanol, hexanol and octanol. Suitable monocarboxylic acids include e.g. benzoic acid, propionic acid and butyric acid. In order to increase the molecular weight and the melt viscosity, the monomers forming the thermoplastic polymer may also include a polyfunctional compound, in case of polycarbonates a polyfunctional alcohol having three or more hydroxyl groups and in case of polyesters a polyfunctional alcohol having three or more hydroxyl groups or a polyfunctional carboxylic acid having three or more carboxyl groups. Suitable polyfunctional alcohols are e.g. glycerine, trimethylol propane, pentaerythrit and 1,3,5-trihydroxy pentane. Suitable polyfunctional carboxylic acids having three or more carboxyl groups are e.g. trimellitic acid and pyromellitic acid. The total amount of these compounds, will frequently not exceed 10 mol-%, based on the molar amount of the diol component.

Suitable carbonate forming monomers, are those, which are conventionally used as carbonate forming monomers in the preparation of polycarbonates, include, but are not limited to phosgene, diphosgene and diester carbonates such as diethyl carbonate, diphenyl carbonate, di-p-tolyl carbonate, phenyl-p-tolyl carbonate, di-p-chlorophenyl carbonate and dinaphthyl carbonate. Out of these, diphenyl carbonate is particularly preferred. The carbonate forming monomer is frequently used at a ratio of 0.97 to 1.20 mol, and more preferably 0.98 to 1.10 mol, with respect to 1 mol of the dihydroxy compound(s) in total.

Suitable dicarboxylic acids include, but are not limited to
aliphatic dicarboxylic acids such as oxalic acid, malonic acid, succinic acid, glutaric acid, adipic acid, pimelic acid, suberic acid, azelaic acid;
alicyclic dicarboxylic acids such as tricyclo[5.2.1.02,6]decane dicarboxylic acid, cyclohexane-1,4-dicarboxylic acid, decalin-2,6-dicarboxylic acid, and norbornandicarboxylic acid; and
aromatic dicarboxylic acids, such as benzene dicarboxylic acids, specifically phthalic acid, isophthalic acid, 2-methylterephthalic acid or terephthalic acid, and naphthalene dicarboxylic acids, specifically naphthalene-1,3-dicarboxylic acid, naphthalene-1,4-dicarboxylic acid, naphthalene-1,5-dicarboxylic acid, naphthalene-1,6-dicarboxylic acid, naphthalene-1,7-dicarboxylic acid, naphthalene-2,5-dicarboxylic acid, naphthalene-2,6-dicarboxylic acid, 2-[9-(carboxymethyl)fluoren-9-yl]acetic acid (formula DC1), 2-[9-(carboxymethyl)fluoren-9-yl]propionic acid (formula DC2), 2,2′-bis(carboxymethyloxy)-1,1′-binaphthyl (formula DC3) and naphthalene-2,7-dicarboxylic acid.

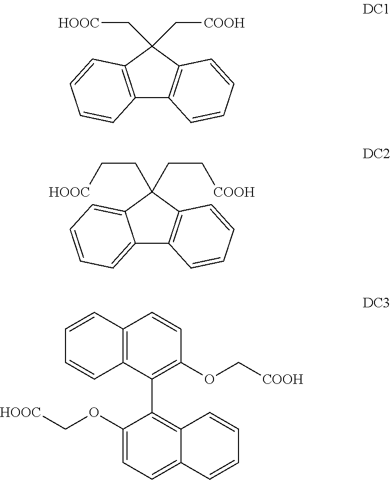

Suitable ester forming derivatives of dicarboxylic acids include, but are not limited to the dialkyl esters, the diphenyl esters and the ditolyl esters.

In case of polyesters, the ester forming monomer is frequently used at a ratio of 0.97 to 1.20 mol, and more preferably 0.98 to 1.10 mol, with respect to 1 mol of the dihydroxy compound(s) in total.

The polycarbonates of the present invention can be prepared by reacting a diol component comprising a monomer of formula (I) and optionally a further diol monomer such as a monomer of the formula (IV) and a carbonate forming monomer by analogy to the well known preparation of polycarbonates as described e.g. in U.S. Pat. No. 9,360,593, US 2016/0319069 and US 2017/0276837, to which full reference is made.

The polyesters of the present invention can be prepared by reacting a diol component comprising a monomer of formula (I) and optionally a further diol monomer such as a monomer of the formula (IV) and a dicarboxylic acid or its ester forming derivative by analogy to the well known preparation of polyesters as described e.g. in US 2017/044311 and the references cited therein, to which full reference is made.

The polyestercarbonates of the present invention can be prepared by reacting a diol component comprising a monomer of formula (I) and optionally a further diol monomer such as a monomer of the formula (IV), a carbonate forming monomer and a dicarboxylic acid or its ester forming derivative by analogy to the well known preparation of polyestercarbonates as described in the art.

The polycarbonates, polyesters and polyestercarbonates are usually prepared by reacting the monomers of the diol component with the carbonate forming monomers and/or the ester forming monomers, i.e. the dicarboxylic acids or the ester forming derivatives thereof, in the presence of an esterification catalyst, in particular a transesterification catalyst, in case a carbonate forming monomer or an ester forming derivative of a polycarboxylic acid is used.

Suitable transesterification catalysts are basic compounds, which specifically include but are not limited to alkaline metal compounds, alkaline earth metal compound, nitrogen-containing compounds, and the like. Likewise, suitable transesterification catalysts are acidic compounds, which specifically include but are not limited to Lewis acid compounds of polyvalent metals, including compounds such as zinc, tin, titanium, zirconium, lead, and the like.

Examples of suitable alkaline metal compound include alkaline metal salts of an organic acid such as acetic acid, stearic acid, benzoic acid, or phenylphorsphoric acid, alkaline metal phenolates, alkaline metal oxides, alkaline metal carbonates, alkaline metal borohydrides, alkaline metal hydrogen carbonates, alkaline metal phosphate, alkaline metal hydrogenphosphate, alkaline metal hydroxides, alkaline metal hydrides, alkaline metal alkoxides, and the like. Specific examples thereof include sodium hydroxide, potassium hydroxide, cesium hydroxide, lithium hydroxide, sodium hydrogen carbonate, sodium carbonate, potassium carbonate, cesium carbonate, lithium carbonate, sodium acetate, potassium acetate, cesium acetate, lithium acetate, sodium stearate, potassium stearate, cesium stearate, lithium stearate, sodium borohydride, sodium borophenoxide, sodium benzoate, potassium benzoate, cesium benzoate, lithium benzoate, disodium hydrogen phosphate, dipotassium hydrogen phosphate, dilithium hydrogen phosphate, and disodium phenylphosphate; and also include disodium salt, dipotassium salt, dicesium salt, dilithium salt of bisphenol A, sodium salt, potassium salt, cesium salt and lithium salt of phenol; and the like.

Examples of the alkaline earth metal compound include alkaline earth metal salts of an organic acid such as acetic acid, stearic acid, benzoic acid, or phenylphorsphoric acid, alkaline earth metal phenolates, alkaline earth metal earth oxides, alkaline earth metal carbonates, alkaline metal borohydrides, alkaline earth metal hydrogen carbonates, alkaline earth metal hydroxides, alkaline earth metal hydrides, alkaline earth metal alkoxides, and the like. Specific examples thereof include magnesium hydroxide, calcium hydroxide, strontium hydroxide, barium hydroxide, magnesium hydrogen carbonate, calcium hydrogen carbonate, strontium hydrogen carbonate, barium hydrogen carbonate, magnesium carbonate, calcium carbonate, strontium carbonate, barium carbonate, magnesium acetate, calcium acetate, strontium acetate, barium acetate, magnesium stearate, calcium stearate, calcium benzoate, magnesium phenylphosphate, and the like.

Examples of the nitrogen-containing compound include quaternary ammoniumhydroxide, salt thereof, amines, and the like. Specific examples thereof include quaternary ammoniumhydroxides including an alkyl group, an aryl group or the like, such as tetramethylammoniumhydroxide, tetraethylammoniumhydroxide, tetrapropylammoniumhydroxide, tetrabutylammoniumhydroxide, trimethylbenzylammoniumhydroxide, and the like; tertiary amines such as triphenylamine, dimethylbenzylamine, triphenylamine, and the like; secondary amines such as diethylamine, dibutylamine, and the like; primary amines such as propylamine, butylamine, and the like; imidazoles such as 2-methylimidazole, 2-phenylimidazole, benzoimidazole, and the like; bases or basic salts such as ammonia, tetramethylammoniumborohydride, tetrabutylammoniumborohydride, tetrabutylammoniumtetraphenylborate, tetraphenylammoniumtetraphenylborate, and the like.

Preferred examples of the transesterification catalyst include salts of polyvalent metals such as zinc, tin, titanium, zirconium, lead, and the like, in particular the chlorides, alkoxyides, alkanoates, benzoates, acetylacetonates and the like. They may be used independently or in a combination of two or more. Specific examples of such transesterification catalyst include zinc acetate, zinc benzoate, zinc 2-ethylhexanoate, tin chloride (II), tin chloride (IV), tin acetate (II), tin acetate (IV), dibutyltinlaurate, dibutyltinoxide, dibutyltinmethoxide, zirconiumacetylacetonate, zirconium oxyacetate, zirconiumtetrabutoxide, lead acetate (II), lead acetate (IV), and the like.

The transesterification catalyst are frequently used at a ratio of 10⁻⁹ to 10⁻³ mol, preferably 10⁻⁷ to 10⁻⁴ mol, with respect to 1 mol of the dihydroxy compound(s) in total.

Frequently, the polycarbonates, polyesters and polyestercarbonates are prepared by a melt polycondensation method. In the melt polycondensation the monomers are reacted in the absence of an additional inert solvent. While the reaction is performed any byproduct formed in the transesterification reaction is removed by heating the reaction mixture at ambient pressure or reduced pressure.

The melt polycondensation reaction preferably comprises charging the monomers and catalyst into a reactor and subjecting the reaction mixture to conditions, where the reaction between the monomers and the formation of the byproduct takes place. It has been found advantageous, if the byproduct resides for at least a while in the polycondensation reaction. However, in order to drive the polycondensation reaction to the product side, it is beneficial to remove at least a portion of the formed byproduct during or preferably at the end of the polycondensation reaction. In order to allow the byproduct in the reaction mixture, the pressure may be controlled by closing the reactor, or by increasing or decreasing the pressure. The reaction time for this step is 20 minutes or longer and 240 minutes or shorter, preferably 40 minutes or longer and 180 minutes or shorter, and especially preferably 60 minutes or longer and 150 minutes or shorter. In this step, in the case where the byproduct is removed by distillation soon after being generated, the finally obtained thermoplastic resin has a low content of high molecular-weight resin molecules. By contrast, in the case where the byproduct is allowed to reside in the reactor for a certain time, the finally obtained thermoplastic resin has a high content of high molecular-weight resin molecules.

The melt polycondensation reaction may be performed in a continuous system or in a batch system. The reactor usable for the reaction may be of a vertical type including an anchor-type stirring blade, a Maxblend® stirring blade, a helical ribbon-type stirring blade or the like; of a horizontal type including a paddle blade, a lattice blade, an eye glass-type blade or the like; or an extruder type including a screw. A reactor including a combination of such reactors is preferably usable in consideration of the viscosity of the polymerization product.

According to the method for producing the thermoplastic resin, such as a polycarbonate resin, after the polymerization reaction is finished, the catalyst may be removed or deactivated in order to maintain the thermal stability and the hydrolysis stability. A preferred method for deactivating the catalyst is the addition of an acidic substance. Specific examples of the acidic substance include esters such as butyl benzoate and the like; aromatic sulfonates such as p-toluenesulfonic acid and the like; aromatic sulfonic acid esters such as butyl p-toluenesulfonate, hexyl p-toluenesulfonate, and the like; phosphoric acids such as phosphorous acid, phosphoric acid, phosphonic acid, and the like; phosphorous acid esters such as triphenyl phosphite, monophenyl phosphite, diphenyl phosphite, diethyl phosphite, di-n-propyl phosphite, di-n-butyl phosphite, di-n-hexyl phosphite, dioctyl phosphite, monooctyl phosphite, and the like; phosphoric acid esters such as triphenyl phosphate, diphenyl phosphate, monophenyl phosphate, dibutyl phosphate, dioctyl phosphate, monooctyl phosphate, and the like; phosphonic acids such as diphenyl phosphonic acid, dioctyl phosphonic acid, dibutyl phosphonic acid, and the like; phosphonic acid esters such as diethyl phenylphosphonate, and the like; phosphines such as triphenylphosphine, bis(diphenylphosphino)ethane, and the like; boric acids such as boric acid, phenylboric acid, and the like; aromatic sulfonic acid salts such as tetarabutylphosphonium dodecylbenzensulfonate salt, and the like; organic halides such as chloride stearate, benzoyl chloride, chloride p-toluenesulfonate, and the like; alkylsulfonic acids such as dimethylsulfonic acid, and the like; organic halides such as benzyl chloride, and the like. These deactivators are frequently used at 0.01 to 50 mol, preferably 0.3 to 20 mol, with respect to the catalyst. After the catalyst has been deactivated, there may be a step of removing low boiling point compounds from the polymer by distillation. The distillation is preferably performed at reduced pressure, e.g. at a pressure of 0.1 to 1 mm Hg at a temperature of 200 to 350° C. For this step, a horizontal device including a stirring blade having a high surface renewal capability such as a paddle blade, a lattice blade, an eye glass-type blade or the like, or a thin film evaporator is preferably used.

It is desirable that the thermoplastic resin such as a polycarbonate resin has a very small amount of foreign objects. Therefore, the molten product is preferably filtered to remove any solids from the melt. The mesh of the filter is preferably 5 μm or less, and more preferably 1 μm or less. It is preferred that the generated polymer is filtrated by a polymer filter. The mesh of the polymer filter is preferably 100 μm or less, and more preferably 30 μm or less. A step of sampling a resin pellet needs to be performed in a low dust environment, needless to say. The dust environment is preferably of class 6 or lower, and more preferably of class 5 or lower.

The thermoplastic resin may be molded by any conventional molding procedure for producing optical elements. Suitable molding procedures include but are not limited to injection molding, compression molding, casting, roll processing, extrusion molding, extension and the like.

While it is possible to mold the thermoplastic resin of the invention as such, it is also possible to mold a resin composition, which contains at least one thermoplastic resin of the invention and which further contains at least one additive and/or further resin. Suitable additives include antioxidants, processing stabilizers, photostabilizers, polymerization metal deactivators, flame retardants, lubricants, antistatic agents, surfactants, antibacterial agents, releasing agents, ultraviolet absorbers, plasticizers, compatibilizers, and the like. Suitable further resins are e.g. another polycarbonate resin, polyester carbonate resin, polyester resin, polyamide, polyacetal and the like, which does not contain repeating units of the formula (I).

Examples of the antioxidant include but are not limited to triethyleneglycol-bis[3-(3-tertbutyl-5-methyl-4-hydroxyphenyl)propionate], 1,6-hexanediol-bis[3-(3,5-di-tert-butyl-4-hydroxyphenyl)propionate], pentaerythritol-tetrakis[3-(3,5-di-tert-butyl-4-hydroxyphenyl)propionate], octadecyl-3-(3,5-di-tert-butyl-4-hydroxyphenyl)propionate, 3,9-bis(2,6-di-tert-butyl-4-methylphenoxy)-2,4,8,10-tetraoxa-3,9-diphosphaspiro[5.5]undecane, 5,7-Di-tert-butyl-3-(3,4-dimethylphenyl)benzofuran-2(3H)-one, 5,7-Di-tert-butyl-3-(1,2dimethylphenyl)benzofuran-2(3H)-one, 1,3,5-trimethyl-2,4,6-tris(3,5-di-tert-butyl-4-hydroxybenzyl)benzene, N,N-hexamethylenebis(3,5-di-tert-butyl-4-hydroxy-hydrocinnamide, 3,5-di-tert-butyl-4-hydroxy-benzylphosphonate-diethylester, tris(3,5-di-tert-butyl-4-hydroxybenzyl)isocyanurate, and 3,9-bis{1,1-dimethyl-2-[β-(3-tert-butyl-4-hydroxy-5-methylphenyl)propionyloxy]ethyl}-2,4,8,10-tetraoxaspiro(5,5)undecane, and the like. Among these examples, 3,9-bis(2,6-di-tert-butyl-4-methylphenoxy)-2,4,8,10-tetraoxa-3,9-diphosphaspiro[5.5]undecane, 5,7-Di-tert-butyl-3-(3,4-dimethylphenyl)benzofuran-2(3H)-one, and 5,7-Di-tert-butyl-3-(1,2dimethylphenyl)benzofuran-2(3H)-one are more preferred. The content of the antioxidant in the thermoplastic resin is preferably 0.001 to 0.3 parts by weight with respect to 100 parts by weight of the thermoplastic resin.

Examples of the processing stabilizer include but are not limited to phosphorus-based processing stabilizers, sulfur-based processing stabilizers, and the like. Examples of the phosphorus-based processing stabilizer include phosphorous acid, phosphoric acid, phosphonous acid, phosphonic acid, esters thereof, and the like. Specific examples thereof include triphenylphosphite, tris(nonylphenyl)phosphite, tris(2,4-di-tert-butylphenyl)phosphite, tris(2,6-di-tert-butylphenyl)phosphite, tridecylphosphite, trioctylphosphite, trioctadecylphosphite, didecylmonophenylphosphite, dioctylmonophenylphosphite, diisopropylmonophenylphosphite, monobutyldiphenylphosphite, monodecyldiphenylphosphite, monooctyldiphenylphosphite, bis(2,6-di-tert-butyl-4-methylphenyl)pentaerythritoldiphosphite, 2,2-methylenebis(4,6-di-tert-butylphenyl)octylphosphite, bis(nonylphenyl)pentaerythritoldiphosphite, bis(2,4-dicumylphenyl)pentaerythritoldiphosphite, bis(2,4-di-tert-butylphenyl)pentaerythritoldiphosphite, distearylpentaerythritoldiphosphite, tributylphosphate, triethylphosphate, trimethylphosphate, triphenylphosphate, diphenylmonoorthoxenylphosphate, dibutylphosphate, dioctylphosphate, diisopropylphosphate, dimethyl benzenephosphonate, diethyl benzenephosphonate, dipropyl benzenephosphonate, tetrakis(2,4-di-t-butylphenyl)-4,4′-biphenylenediphosphonite, tetrakis(2,4-di-t-butylphenyl)-4,3′-biphenylenediphosphonite, tetrakis(2,4-di-t-butylphenyl)-3,3′-biphenylenediphosphonite, bis(2,4-di-tert-butylphenyl)-4-phenyl-phenylphosphonite, bis(2,4-di-tert-butylphenyl)-3-phenyl-phenylphosphonite, and the like. The content of the phosphorus-based processing stabilizer in the thermoplastic resin composition is preferably 0.001 to 0.2 parts by weight with respect to 100 parts by weight of the thermoplastic resin.

Examples of the sulfur-based processing stabilizer include but are not limited to pentaerythritol-tetrakis(3-laurylthiopropionate), pentaerythritol-tetrakis(3-myristylthiopropionate), pentaerythritol-tetrakis(3-stearylthiopropionate), dilauryl-3,3′-thiodipropionate, dimyristyl-3,3′-thiodipropionate, distearyl-3,3′-thiodipropionate, and the like. The content of the sulfur-based processing stabilizer in the thermoplastic resin composition is preferably 0.001 to 0.2 parts by weight with respect to 100 parts by weight of the thermoplastic resin.

Preferred releasing agents contain at least 90% by weight of an ester of an alcohol and a fatty acid. Specific examples of the ester of an alcohol and a fatty acid include an ester of a monovalent alcohol and a fatty acid, and a partial ester or a total ester of a polyvalent alcohol and a fatty acid. Preferred examples of the above-described ester of an alcohol and a fatty acid include the esters of a monovalent alcohol having a carbon number of 1 to 20 and a saturated fatty acid having a carbon number of 10 to 30. Preferred examples of partial or total esters of a polyvalent alcohol and a fatty acid include the partial or total ester of a polyvalent alcohol having a carbon number of 2 to 25 and a saturated fatty acid having a carbon number of 10 to 30. Specific examples of the ester of a monovalent alcohol and a fatty acid include stearyl stearate, palmityl palmitate, butyl stearate, methyl laurate, isopropyl palmitate, and the like. Specific examples of the partial or total ester of a polyvalent alcohol and a fatty acid include monoglyceride stearate, monoglyceride stearate, diglyceride stearate, triglyceride stearate, monosorbitate stearate, monoglyceride behenate, monoglyceride caprylate, monoglyceride laurate, pentaerythritol monostearate, pentaerythritol tetrastearate, pentaerythritol tetrapelargonate, propyleneglycol monostearate, biphenyl biphenate, sorbitan monostearate, 2-ethylhexylstearate, total or partial esters of dipentaerythritol such as dipentaerythritol hexastearate and the like, etc. The content of the releasing agent in the resin composition is preferably 0.005 to 2.0 parts by weight, more preferably 0.01 to 0.6 parts by weight, and still more preferably 0.02 to 0.5 parts by weight, with respect to 100 parts by weight of the thermoplastic resin.

Preferred ultraviolet absorbers are selected from the group consisting of benzotriazole-based ultraviolet absorbers, benzophenone-based ultraviolet absorbers, triazine-based ultraviolet absorbers, cyclic iminoester-based ultraviolet absorbers, and cyanoacrylate-based ultraviolet absorbers. Namely, the following ultraviolet absorbers may be used independently or in a combination of two or more.

Examples of benzotriazole-based ultraviolet absorbers include 2-(2-hydroxy-5-methylphenyl)benzotriazole, 2-(2-hydroxy-5-tert-octylphenyl)benzotriazole, 2-(2-hydroxy-3,5-dicumylphenyl)phenylbenzotriazole, 2-(2-hydroxy-3-tert-butyl-5-methylphenyl)-5-chlorobenzotriazole, 2,2′-methylenebis[4-(1,1,3,3-tetramethylbutyl)-6-(2N-benzotriazole-2-yl)phenol)], 2-(2-hydroxy-3,5-di-tert-butylphenyl)benzotriazole, 2-(2-hydroxy-3,5-di-tert-butylphenyl)-5-chlorobenzotriazole, 2-(2-hydroxy-3,5-di-tert-amylphenyl)benzotriazole, 2-(2-hydroxy-5-tert-octylphenyl)benzotriazole, 2-(2-hydroxy-5-tert-butylphenyl)benzotriazole, 2-(2-hydroxy-4-octoxyphenyl)benzotriazole, 2,2′-methylenebis(4-cumyl-6-benzotriazolephenyl), 2,2′-p-phenylenebis(1,3-benzoxazine-4-one), 2-[2-hydroxy-3-(3,4,5,6-tetrahydrophthalimidemethyl)-5-methylphenyl]benzotriazole, and the like.

Examples of benzophenone-based ultraviolet absorbers include 2,4-dihydroxybenzophenone, 2-hydroxy-4-methoxybenzophenone, 2-hydroxy-4-octoxybenzophenone, 2-hydroxy-4-benzyloxybenzophenone, 2-hydroxy-4-methoxy-5-sulfoxybenzophenone, 2-hydroxy-4-methoxybenzophenone-5-sulfonic acid hydrate, 2,2′-dihydroxy-4-methoxybenzophenone, 2,2′,4,4′-tetrahydroxybenzophenone, 2,2′-dihydroxy-4,4′-dimethoxybenzophenone, 2,2′-dihydroxy-4,4′-dimethoxy-5-sodiumsulfoxybenzophenone, bis(5-benzoyl-4-hydroxy-2-methoxyphenyl)methane, 2-hydroxy-4-n-dodecyloxybenzophenone, 2-hydroxy-4-methoxy-2′-carboxybenzophenone, and the like.

Examples of triazine-based ultraviolet absorbers include 2-(4,6-diphenyl-1,3,5-triazine-2-yl)-5-([(hexyl)oxy]-phenol, 2-(4,6-bis(2,4-dimethylphenyl)-1,3,5-triazine-2-yl)-5-([(octyl)oxy]-phenol, and the like.

Examples of cyclic iminoester-based ultraviolet absorbers include 2,2′-bis(3,1-benzoxazine-4-one), 2,2′-p-phenylenebis(3,1-benzoxazine-4-one), 2,2′-m-phenylenebis(3,1-benzoxazine-4-one), 2,2′-(4,4′diphenylene)bis(3,1-benzoxazine-4-one), 2,2′-(2,6-naphthalene)bis(3,1-benzoxazine-4-one), 2,2′-(1,5-naphthalene)bis(3,1-benzoxazine-4-one), 2,2′-(2-methyl-p-phenylene)bis(3,1-benzoxazine-4-one), 2,2′-(2-nitro-p-phenylene)bis(3,1-benzoxazine-4-one), 2,2′-(2-chloro-p-phenylene)bis(3,1-benzoxazine-4-one), and the like.

Examples of cyanoacrylate-based ultraviolet absorbers include 1,3-bis-[(2′-cyano-3′,3′-diphenylacryloyl)oxy]-2,2-bis(((2-cyano-3,3-diphenylacryloyl)oxy)methyl)propane, 1,3-bis-[(2-cyano-3,3-diphenylacryloyl)oxy]benzene, and the like.

The content of the ultraviolet absorber in the resin composition is preferably 0.01 to 3.0 parts by weight, more preferably 0.02 to 1.0 parts by weight, and still more preferably 0.05 to 0.8 parts by weight, with respect to 100 parts by weight of the thermoplastic resin. The ultraviolet absorber contained in such a range of content in accordance with the use may provide a sufficient climate resistance to the thermoplastic resin.

As mentioned above, the thermoplastic polymer resins, in particular the polycarbonate resins, comprising repeating units of formulae (II), (IIa) and (IIb), respectively, as described herein, provide high transparency and high refractive index to thermoplastic resins, which therefore are suitable for preparing optical devices, where high transparency and high refractive index is required. More precisely, the thermoplastic polycarbonates having structural units of formulae (II), (IIa) and (IIb), respectively, are characterized by having a high refractive index, which is preferably at least 1.640, more preferably at least 1.660, in particular at least 1.670.

The contribution of the monomer of the formulae (I), (Ia) and (Ib), respectively, to the refractive index of the thermoplastic resin, in particular a polycarbonate resin, will depend from the refractive index of said monomer and the relative amount of said monomer in the thermoplastic resin. In general, a higher refractive index of the monomer contained in the thermoplastic resin will result in a higher refractive index of the resulting thermoplastic resin. Apart from that, the refractive index of a thermoplastic resin comprising structural units of the formula (11) can be calculated from the refractive indices of the monomers used for preparing the thermoplastic resin, either from the refractive index of the monomers or ab initio, e.g. by using the computer software ACD/ChemSketch 2012 (Advanced Chemistry Development, Inc.).

In case of thermoplastic copolymer resins, the refractive index of the thermoplastic resin, in particular a polycarbonate resin, can be calculated from the refractive indices of the homopolymers of the respective monomers, which form the copolymer resin, by the following so called “Fox equation”:

$$1 / {n}_{D} = {x}_{1} / {n}_{D 1} + {x}_{2} / {n}_{D 2} + … . {x}_{n} / {n}_{Dn} ,$$

where n_{D} is the refractive index of the copolymer, x₁, x₂, . . . xₙ are the mass fractions of the monomers 1, 2, . . . n in the copolymer and n_{D1}, n_{D2}, . . . n_{Dn} are the refractive indices of the homopolymers synthesized from only one of the monomers 1, 2, . . . n at a time. In case of polycarbonates, x₁, x₂, . . . xₙ are the mass fractions of the OH monomers 1, 2, . . . n, based on the total amount of OH monomer. It is apparent that a higher refractive index of a homopolymer will result in a higher refractive index of the copolymer.

The refractive indices of the thermoplastic resins can be determined directly or indirectly. For direct determination, the refractive indices n_{D} of the thermoplastic resins are measured at wavelength of 589 nm in accordance with the protocol JIS-K-7142 using an Abbe refractometer and applying a 0.1 mm film of the thermoplastic resin. In case of the refractive indices of the homopolycarbonates of the compounds of formula (I), the refractive indices can also be determined indirectly. For this, a co-polycarbonate of the respective monomer of formula (I) with 9,9-bis(4-(2-hydroxyethoxy)phenyl)fluorene and diphenyl carbonate is prepared according to the protocol of example 1 in column 48 of U.S. Pat. No. 9,360,593 and the refractive indices n_{D} of the co-polycarbonate is measured at wavelength of 589 nm in accordance with the protocol JIS-K-7142 using an Abbe refractometer and applying a 0.1 mm film of the co-polycarbonate. From the thus measured refractive indices no, the refractive index of the homopolycarbonate of the respective monomer can be calculated by applying the Fox equation and the known refractive index of 9,9-bis(4-(2-hydroxyethoxy)phenyl)fluorene (n_{D}(589 nm)=1.639).

The compounds of formula (I) can be obtained in a purity, which provides for a low yellowness index Y.I., as determined in accordance with ASTM E313, which may also be important for the use in the preparation of optical resins.

More precisely, the yellowness index Y.I., as determined in accordance with ASTM E313, of the compounds of formula (I) preferably does not exceed 100, more preferably 50, even more preferably 20, in particular 10 or 5.

The thermoplastic resin according to the present invention has a high refractive index and a low Abbe number. The thermoplastic resin of the present invention can be used for producing a transparent conductive substrate usable for a liquid crystal display, an organic EL display, a solar cell and the like. Also, the thermoplastic resin of the present invention can be used as a structural material for optical parts, such as, optical disks, liquid crystal panels, optical cards, optical sheets, optical fibers, connectors, evaporated plastic reflecting mirrors, displays, and the like; or used as optical devices suitable for functional material purpose.

Accordingly, molded articles, such as optical devices can be formed using the thermoplastic resins of the present invention. The optical devices include optical lenses, and optical films. The specific examples of the optical devices include lenses, films, mirrors, filters, prisms, and so on. These optical devices can be formed by arbitrary production process, for example, by injection molding, compression molding, injection compression molding, extrusion molding, or solution casting.

Because of an excellent moldability and a high heat resistance, the thermoplastic resins of the present invention are very suitable for production of optical lenses which requires injection molding. For molding, the thermoplastic resins of the present invention, such as the polycarbonate resin, can be used with other thermoplastic resins, for example, different polycarbonate resin, polyestercarbonate resin, polyester resin, and other resins, as a mixture.

In addition, the thermoplastic resins of the present invention can be mixed with additives for forming the optical devices. As the additives for forming the optical devices, above-mentioned ones can be used. The additives may include antioxidants, processing stabilizers, photostabilizers, polymerization metal deactivators, flame retardants, lubricants, antistatic agents, surfactants, antibacterial agents, releasing agents, ultraviolet absorbers, plasticizers, compatibilizers, and the like.

As is clear from the above, another aspect of the present invention relates to an optical device made of a thermoplastic resin as defined above, where the thermoplastic resin comprising a structural unit represented by the formula (II) and optionally of formula (V). As regards to the preferred meanings and preferred embodiments of the structural units of the formulae (II) and (V), reference is made to the statements given above.

An optical device made of an optical resin comprising the repeating units of the formula (II) and optionally repeating units of the formula (V) as defined herein are usually optical molded articles such as optical lenses, for example car head lamp lenses, Fresnel lenses, fθ lenses for laser printers, camera lenses, lenses for glasses and projection lenses for rear projection TV's, CD-ROM pick-up lenses, but also optical disks, optical elements for image display media, optical films, film substrates, optical filters or prisms, liquid crystal panels, optical cards, optical sheets, optical fibers, optical connectors, eposition plastic reflective mirrors, and the like. Here particular preference is given to optical lenses and optical films. Optical resins comprising repeating units of the formula (II) and optionally repeating units of the formula (V) are also useful for producing a transparent conductive substrate usable for an optical device suitable as a structural member or a functional member of a transparent conductive substrate for a liquid crystal display, an organic EL display, a solar cell and the like.

The optical lens produced from the thermoplastic resin according to the present invention has a high refractive index, a low Abbe number and a low degree of birefringence, and is highly moisture and heat resistant. Therefore, the optical lens can be used in the field in which a costly glass lens having a high refractive index is conventionally used, such as for a telescope, binoculars, a TV projector and the like. It is preferred that the optical lens is used in the form of an aspherical lens. Merely one aspherical lens may make the spherical aberration substantially zero. Therefore, it is not necessary to use a plurality of spherical lenses to remove the spherical aberration. Thereby the weight and the production cost of a device including the spherical aberration is decreased. An aspherical lens is useful especially as a camera lens among various types of optical lenses. The present invention easily provides an aspherical lens having a high refractive index and a low level of birefringence, which is technologically difficult to produce by processing glass.

An optical lens of the present invention may be formed, for example, by injection molding, compression molding, injection compression molding or casting the resin the repeating units of the formula (II) and optionally repeating units of the formula (V) as defined herein.

The optical lens of the present invention is characterized by a small optical distortion. An optical lens comprising a conventional optical resin has a large optical distortion. Although it is not impossible to reduce the value of an optical distortion by molding conditions, the condition widths are very small, thereby making molding extremely difficult. Since the resin having repeating units of the formula (II) and optionally repeating units of the formula (V) as defined herein has an extremely small optical distortion caused by the orientation of the resin and a small molding distortion, an excellent optical element can be obtained without setting molding conditions strictly.

To manufacture the optical lens of the present invention by injection molding, it is preferred that the lens should be molded at a cylinder temperature of 260° C. to 320° C. and a mold temperature of 100° C. to 140° C.

The optical lens of the present invention is advantageously used as an aspherical lens as required. Since spherical aberration can be substantially nullified with a single aspherical lens, spherical aberration does not need to be removed with a combination of spherical lenses, thereby making it possible to reduce the weight and the production cost. Therefore, out of optical lenses, the aspherical lens is particularly useful as a camera lens.

Since resins having repeating units of the formula (II) and optionally repeating units of the formula (V) as defined herein have a high moldability, they are particularly useful as the material of an optical lens, which is thin and small in size and has a complex shape. As a lens size, the thickness of the center part of the lens is 0.05 to 3.0 mm, preferably 0.05 to 2.0 mm, more preferably 0.1 to 2.0 mm. The diameter of the lens is 1.0 to 20.0 mm, preferably 1.0 to 10.0 mm, more preferably 3.0 to 10.0 mm. It is preferably a meniscus lens, which is convex on one side and concave on the other side.

The surface of the optical lens of the present invention may have a coating layer such as an antireflection layer or a hard coat layer as required. The antireflection layer may be a single layer or a multi-layer and composed of an organic material or inorganic material but preferably an inorganic material. Examples of the inorganic material include oxides and fluorides such as silicon oxide, aluminum oxide, zirconium oxide, titanium oxide, cerium oxide, magnesium oxide and magnesium fluoride.

The optical lens of the present invention may be formed by an arbitrary method such as metal molding, cutting, polishing, laser machining, discharge machining or edging. Metal molding is preferred.

An optical film produced by the use of the thermoplastic resin according to the present invention is high in transparency and heat resistance, and therefore is preferably usable for a liquid crystal substrate film, an optical memory card or the like. In order to avoid foreign objects from being incorporated into the optical film as much as possible, the molding needs to be performed in a low dust environment, needless to say. The dust environment is preferably of class 6 or lower, and more preferably of class 5 or lower.

The following examples serve as further illustration of the invention.

### 1. Abbreviations

m.p.: melting point
eq.: molar equivalent(s)
DMF: dimethylformamide
HCl: hydrochloric acid
K₂CO₃: potassium carbonate
KI: potassium iodide
KOH: potassium hydroxide
MTBE: methyl tert-butyl ether
NaCl: sodium chloride
Na₂SO₄: sodium sulfate
NaHCO₃: sodium hydrogencarbonate
NH₄Cl: ammonium chloride
THF: tetrahydrofuran
TLC: thin layer chromatography
n_{D}: Refractive index
ν: Abbe number
Mw: Molecular weight
Tg: Glass transition temperature
CLWC: Contents of low molecular weight compounds
GPC: Gel permeation chromatography
BPEF: 9,9-bis[4-(2-hydroxyethoxy)phenyl]fluorine
BNEF: 9,9-bis[6-(2-hydroxyethoxy)naphthalene-2-yl]fluorene
BNE: 2,2′-bis(2-hydroxyethoxy)-1,1′-binaphtyl
DPC: diphenylcarbonate

### 2. Preparation of Monomers of Formula (I)

### 2.1 Analytics Relating to Monomers of Formula (I)

¹H-NMR spectra were determined at 23° C. using a 80 MHz NMR-spectrometer (Magritek Spinsolve 80). If not stated otherwise the solvent was CDCl₃.

Melting points of the compounds were determined by Buchi Melting Point B-545.

### 2.2 Preparation Examples

### Example 1a: 2′,2″-[1,4-phenylenebis(methyleneoxy)]di([1,1′-binaphthalen]-2-ol) (DBNABHP) (compound of formula (Ia), with X=hydrogen and A¹=1,4-phenylene; compound 1 of table A)

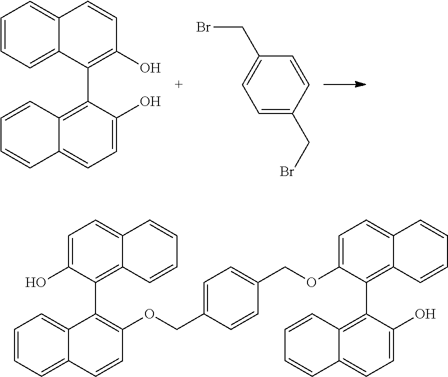 [SMILES: Oc1ccc2ccccc2c1-c1c(O)ccc2ccccc12 ; BrCc1ccc(CBr)cc1 ; Oc1ccc2ccccc2c1-c1c(OCc2ccc(COc3ccc4ccccc4c3-c3c(O)ccc4ccccc34)cc2)ccc2ccccc12]

To a mixture of rac. 1,1′-bi-2-naphthol (111 g, 388 mmol, 2.05 eq.) and K₂CO₃ (152 g, 1.10 mol, 5.8 eq.) in acetone (1.20 kg) was added dropwise a solution of 1,4-bis(bromomethyl)benzene (50.0 g, 189 mmol, 1.0 eq.) in acetone (1.00 kg) at 60° C. The reaction mixture was stirred at 60° C. until TLC control (cyclohexane/ethyl acetate 2:1) showed complete conversion.

The reaction was quenched by addition of water (1.00 kg) and the acetone removed under reduced pressure. Dichloromethane (500 g) was added to the residue and the aqueous phase acidified by addition of 10% hydrochloric acid (pH=4-5). The phases were separated and the organic phase was washed subsequently with water (200 g), then with saturated aqueous NaCl solution (200 g), dried over Na₂SO₄ and the solvent completely removed under reduced pressure.

The crude product was recrystallized twice from toluene and washed with pentane to give the title compound as a white solid (52.0 g, 77.1 mmol, yield: 41%, chemical purity: 99.3%).

m.p.=170-200° C.

¹H NMR (80 MHz, CDCl₃): δ=8.11-7.71 (m, 8H), 7.53-6.93 (m, 16H), 6.83 (s, 4H), 4.98 (s, 4H), 4.87 (s, 2H) ppm.

### Example 1b: 2′,2″-[1,4-phenylenebis(methyleneoxy)]di([1,1′-binaphthalen]-2-ol) (DBNABHP) (compound of formula (Ia), with X=hydrogen and A, =1,4-phenylene; compound 1 of table A)

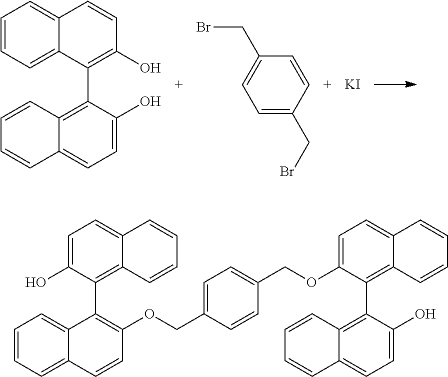

To a mixture of rac. 1,1′-bi-2-naphthol (288 g, 1.01 mol, 2.2 eq.), K₂CO₃ (139 g, 1.01 mol, 2.2 eq.) and KI (15.2 g, 91.4 mmol, 0.2 eq.) in acetone (1.6 kg) was added dropwise a solution of para-α,α′-Dichloro xylene (80.0 g, 457 mmol, 1.0 eq.) in acetone (400 g) at 60° C. over a period of 8 hours. The reaction mixture was stirred at 60° C. until TLC control (cyclohexane/ethyl acetate 2:1) showed complete conversion (approx. 18 hours).

After complete conversion, 2 L of acetone were removed under reduced pressure. Then, water (1 kg) and toluene (1 kg) were added to the reaction mixture and the remaining acetone removed under reduced pressure. The residue was acidified by addition of 10% hydrochloric acid (pH=4-5). The phases were separated at 60° C. and the aqueous phase extracted with toluene (500 g). The combined organic phases were washed with water (500 g), dried over Na₂SO₄ and the solution concentrated to a residual mass of 1 kg. The mixture was cooled to room temperature and stirred for approx. 2 hours. The suspension was diluted with toluene (300 g), the formed crystals were filtered off and washed with pentane to give the crude product as a slightly yellow solid. The crude product was recrystallized twice from toluene to give the title compound as a white solid (184 g, 273 mmol, yield: 60%, chemical purity: 99.3%).

m.p.=170-200° C.

¹H NMR (80 MHz, CDCl₃): δ=8.11-7.71 (m, 8H), 7.53-6.93 (m, 16H), 6.83 (s, 4H), 4.98 (s, 4H), 4.87 (s, 2H) ppm.

### Example 2a: 2,2′-[1,4-phenylenebis(methyleneoxy[1,1′-binaphthalene]-2′,2-diyloxy)]di(ethan-1-ol) (DBHBNABHP) (compound of formula (Ia), with X=2-hydroxyethyl and A¹=1,4-phenylene; compound 25 of Table A)

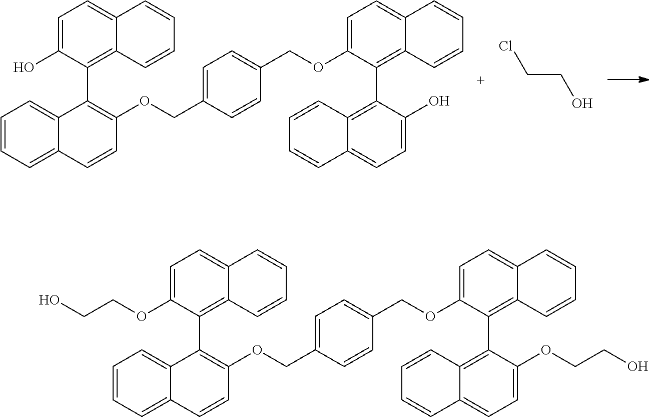

To a solution of di(2′-hydroxy-1,1′-binaphthyl) bishydroxymethylbenzene (34.0 g, 50.4 mmol, 1.0 eq.) in DMF (280 g) was added K₂CO₃ (27.9 g, 202 mol, 4.0 eq.) and the reaction was heated to 50° C. for 30 minutes. 2-Chloroethanol (16.2 g, 202 mmol, 4.0 eq.) was added at 50° C. and the reaction was heated to 130° C. until TLC (cyclohexane/ethyl acetate 1:1) showed complete conversion.

The reaction was cooled below 100° C., then water (750 g) was added and the mixture extracted at room temperature with dichloromethane. The organic phase was dried over Na₂SO₄ and the solvent completely removed under reduced pressure to give the title compound as a white solid (35.0 g, 45.9 mmol, yield: 91%, chemical purity: 97.8%).

¹H NMR (80 MHz, CDCl₃): δ=8.07-7.72 (m, 8H), 7.49-6.99 (m, 16H), 6.62 (s, 4H), 4.89 (m, 4H), 4.16-3.91 (m, 4H), 3.62-3.32 (m, 4H), 1.95 (app. dt, J=6.6, 2.1 Hz, 2H) ppm.

### Example 2b: 2,2′-[1,4-phenylenebis(methyleneoxy[1,1′-binaphthalene]-2′,2-diyloxy)]di(ethan-1-ol) (DBHBNABHP) (compound of formula (Ia), with X=2-hydroxyethyl and A¹=1,4-phenylene; compound 25 of Table A)

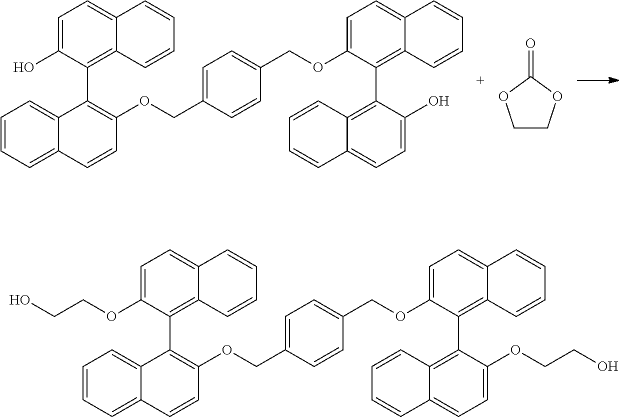 [SMILES: Oc1ccc2ccccc2c1-c1c(OCc2ccc(COc3ccc4ccccc4c3-c3c(O)ccc4ccccc34)cc2)ccc2ccccc12 ; O=C1OCCO1 ; OCCOc1ccc2ccccc2c1-c1c(OCc2ccc(COc3ccc4ccccc4c3-c3c(OCCO)ccc4ccccc34)cc2)ccc2ccccc12]

A mixture of di(2′-hydroxy-1,1′-binaphthyl) bishydroxymethylbenzene (5.0 g, 7.4 mmol, 1.0 eq.), K₂CO₃ (1.0 g, 7.4 mmol, 1.0 eq.) and KI (0.25 g, 1.5 mmol, 0.2 eq.) in toluene (30 g) was heated to 100° C. A solution of ethylene carbonate (16.2 g, 202 mmol, 4.0 eq.) in toluene (10 g) was added dropwise and the reaction heated to reflux until TLC (cyclohexane/ethyl acetate 1:1) showed complete conversion.

The reaction was cooled to 70° C., then water (10 g) was added and the mixture stirred for 30 min. The aqueous phase was removed, then an 10% aqueous citric acid solution (10 g) added. The mixture was stirred again for 30 min, the aqueous phase removed and 15% aqueous NaOH (10 g) added. The mixture was stirred again for 1 hour, the aqueous phase removed. Water (10 g) was added, the mixture stirred once more for 30 min, the phases separated. The organic phase was dried over Na₂SO₄ and the solvent completely removed under reduced pressure to give the title compound as a slightly yellow solid (5.1 g, 6.7 mmol, yield: 90%, chemical purity: 93.1%). The compound can be further purified by repeated crystallization from toluene or isobutanol to give the product as a white solid with a chemical purity of >95%.

¹H NMR (80 MHz, CDCl₃): δ=8.07-7.72 (m, 8H), 7.49-6.99 (m, 16H), 6.62 (s, 4H), 4.89 (m, 4H), 4.16-3.91 (m, 4H), 3.62-3.32 (m, 4H), 1.95 (app. dt, J=6.6, 2.1 Hz, 2H) ppm.

### Example 2c: 2,2′-[1,4-phenylenebis(methyleneoxy[1,1′-binaphthalene]-2′,2-diyloxy)]di(ethan-1-ol) (DBHBNABHP) (compound of formula (Ia), with X=2-hydroxyethyl and A¹=1,4-phenylene; compound 25 of Table A)

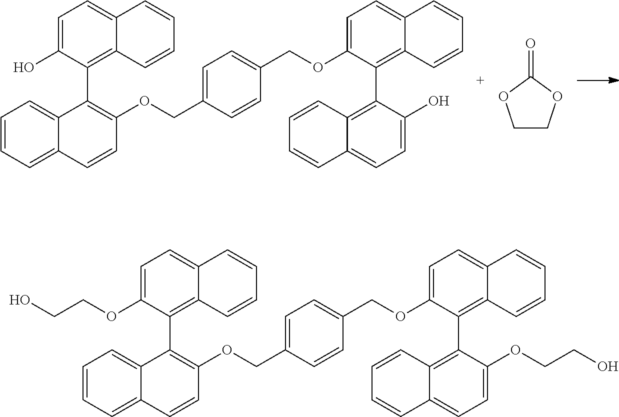 [SMILES: Oc1ccc2ccccc2c1-c1c(OCc2ccc(COc3ccc4ccccc4c3-c3c(O)ccc4ccccc34)cc2)ccc2ccccc12 ; O=C1OCCO1 ; OCCOc1ccc2ccccc2c1-c1c(OCc2ccc(COc3ccc4ccccc4c3-c3c(OCCO)ccc4ccccc34)cc2)ccc2ccccc12]

A mixture of di(2′-hydroxy-1,1′-binaphthyl) bishydroxymethylbenzene (10 g, 14.8 mmol, 1.0 eq.), K₂CO₃ (1.0 g, 7.4 mmol, 0.5 eq.) in toluene (30 g) and DMF (2.5 g) was heated to 100° C. A solution of ethylene carbonate (2.74 g, 31.1 mmol, 2.1 eq.) in toluene (10 g) was added dropwise and the reaction mixture was heated to reflux until TLC (cyclohexane/ethyl acetate 1:1) showed complete conversion.

The reaction was cooled to 70° C., then, methanol (5 g) was added and the mixture was stirred under reflux for 2 h. Then, water (20 g) was added and the mixture was stirred for 30 min. The aqueous phase was removed. To the organic phase was added activated charcoal (200 mg) and the mixture was stirred for 30 min. The mixture was filtered over celite at 60° C. to remove the charcoal. The filtrate was concentrated under reduced pressure to remove methanol and then cooled to room temperature. The crystallized product was isolated by filtration, washed with n-pentane and dried to yield the title compound DBHBNABHP as white crystals (9.2 g; 12 mmol) with a chemical purity of 98.1%.

m.p.=120-150° C.

¹H NMR (80 MHz, CDCl₃): δ=8.07-7.72 (m, 8H), 7.49-6.99 (m, 16H), 6.62 (s, 4H), 4.89 (m_{c}, 4H), 4.16-3.91 (m, 4H), 3.62-3.32 (m, 4H), 1.95 (app. dt, J=6.6, 2.1 Hz, 2H) ppm.

### Example 3: [1,4-Phenylenebis(methyleneoxy[1,1′-binaphthalene]-2′,2-diyloxymethylene-4,1-phenylene)]dimethanol (DMOBBNABHP) (compound of formula (Ia), with X=(4-(hydroxymethyl)phenyl)methyl and A¹=1,4-phenylene; compound 73 of Table A)

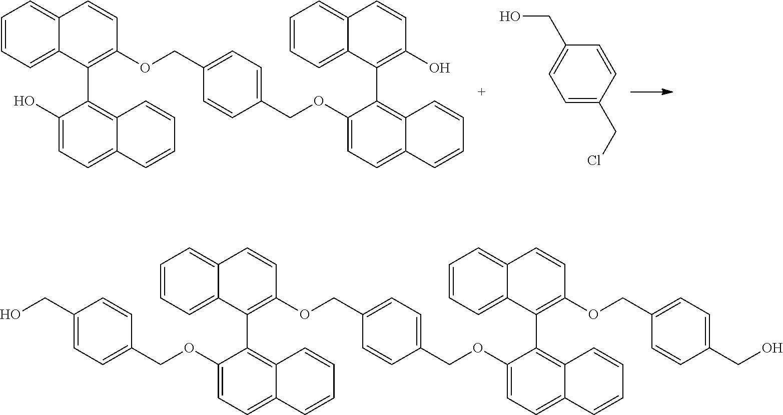 [SMILES: Oc1ccc2ccccc2c1-c1c(OCc2ccc(COc3ccc4ccccc4c3-c3c(O)ccc4ccccc34)cc2)ccc2ccccc12 ; OCc1ccc(CCl)cc1 ; OCc1ccc(COc2ccc3ccccc3c2-c2c(OCc3ccc(COc4ccc5ccccc5c4-c4c(OCc5ccc(CO)cc5)ccc5ccccc45)cc3)ccc3ccccc23)cc1]

To a mixture of DBNABHP (70.0 g, 104 mmol, 1.0 eq.), K₂CO₃ (29.4 g, 213 mmol, 2.05 eq.) and KI (1.72 g, 10.4 mmol, 0.1 eq.) in acetone (450 g) was added 4-chlormethylbenzylalcohol (33.3 g, 213 mmol, 2.05 eq.). The mixture was heated to reflux until TLC control (cyclohexane/ethyl acetate 1:1) showed complete conversion. The reaction mixture was filtered at 40° C. over celite to remove inorganic salts and the solvent was subsequently completely removed under reduced pressure.

The crude product was recrystallized from a mixture of toluene (200 g) and ethyl acetate (10 g) with activated charcoal (2.5 g, Norit DX Ultra) followed by slurry wash in MTBE to give the title compound as a white solid (84.4 g, 92.2 mmol, yield: 89%, chemical purity: 96.9%).

m.p.=92-98° C.

¹H NMR (80 MHz, CDCl₃, ppm): δ=8.02-7.77 (m, 8H), 7.48-7.11 (m, 16H), 7.08-6.75 (m, 8H), 6.65 (s, 4H), 4.95 (s, 4H), 4.90 (s, 4H), 4.48 (s, 4H) ppm.

¹H NMR (80 MHz, DMSO-d₆, ppm): δ=8.18-7.81 (m, 8H), 7.74-6.82 (m, 24H), 6.74 (s, 4H), 5.05 (s, 4H), 5.04 (s, 4H), 5.04 (t, J=4.7 Hz, 2H), 4.37 (d, J=4.7 Hz, 4H) ppm.

### Example 4: Di(2′-hydroxy-1,1′-binaphthyl) bishydroxymethyl-2,6-naphthalene (DBNABHN) (compound of formula (Ia), with X=hydrogen and A¹=2,6-naphthylene; compound 6 of table A)

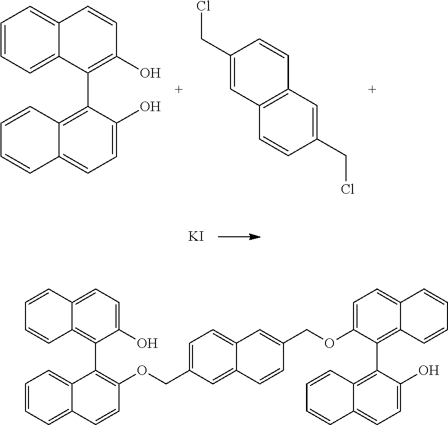 [SMILES: Oc1ccc2ccccc2c1-c1c(O)ccc2ccccc12 ; ClCc1ccc2cc(CCl)ccc2c1 ; "KI" ; Oc1ccc2ccccc2c1-c1c(OCc2ccc3cc(COc4ccc5ccccc5c4-c4c(O)ccc5ccccc45)ccc3c2)ccc2ccccc12]

To a mixture of rac. 1,1′-bi-2-naphthol (39.2 g, 137 mmol, 2.2 eq.), K₂CO₃ (18.9 g, 137 mmol, 2.2 eq.) and KI (2.06 g, 12.4 mmol, 0.2 eq.) in acetone (330 g) was added dropwise a solution of 2,6-di(chloromethyl) naphthalene (14.0 g, 62.2 mmol, 1.0 eq.) in acetone (160 g) at 60° C. The reaction mixture was stirred at 60° C. until TLC control (cyclohexane/ethyl acetate 2:1) showed complete conversion.

Water (140 g) and toluene (140 g) were added to the reaction mixture and the acetone removed under reduced pressure. The residue was acidified by addition of 10% hydrochloric acid (pH=4-5). The phases were separated at 60° C. and the aqueous phase extracted with toluene (40 g). The combined organic phases were washed with water (70 g), dried over Na₂SO₄ and the solution concentrated to a residual mass of 90 g. The mixture was cooled to room temperature and stirred for approx. 2 hours. The formed crystals were filtered off and washed with pentane to give the title compound as a white solid (30.0 g, 41.4 mmol, yield: 67%, chemical purity: 91.5%). The compound can be further purified by repeated crystallization from toluene to give the product with a chemical purity of >95%.

¹H NMR (80 MHz, CDCl₃): δ=8.07-7.71 (m, 8H), 7.55-6.89 (m, 22H), 5.26-4.89 (m, 6H) ppm.

### Example 5: 2,2′-[1,4-Naphthylenebis(methyleneoxy[1,1′-binaphthalene]-2′,2-diyloxy)]di(ethan-1-ol) (DBHBNA14BHN) (compound of formula (Ia), with X=2-hydroxyethyl and A¹=1,4-naphthylene; compound 31 of table A)

### Example 5a: 2-[2-Hydroxy-(1,1′-binaphthalene)-2′-oxy]-ethan-1-ol (building block 1)

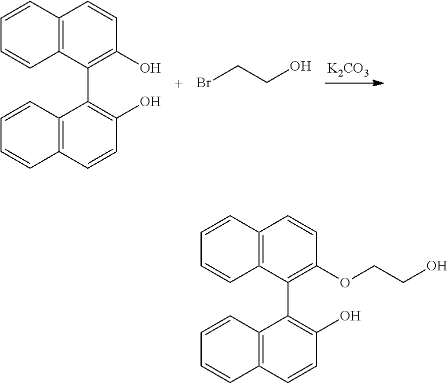

A mixture of rac. 1,1′-bi-2-naphthol (200 g, 692 mmol, 1.0 eq.) and K₂CO₃ (110 g, 795 mmol, 1.15 eq.) in methylethylketone (MEK) (930 g) was heated to reflux for 60 minutes. Then, a solution of 2-bromoethanol (112 g, 899 mmol, 1.3 eq.) in MEK (200 mL) was slowly added and the reaction mixture was stirred at reflux for approximately 7 hours.

The reaction mixture was cooled to room temperature, then toluene (1000 ml) and water (400 ml) were added. The mixture was acidified with aqueous HCl. The phases were separated and the aqueous phase was extracted with toluene. The combined corganic phases were washed with water and brine. The organic phase was concentrated under reduced pressure (150 mbar) until all MEK was removed by distillation. The mixture was cooled to room temperature. The formed crystals were filtered off to give the title compound as a white solid (80.0 g, 242 mmol, yield: 35%, chemical purity: 83.75%). Repeated slurry wash in THE gave the desired product with a chemical purity of >99%.

¹H NMR (80 MHz, CDCl₃): δ=8.16-7.75 (m, 4H), 7.56-6.96 (m, 8H), 5.05 (br s, 1H), 4.23-4.03 (m, 2H), 3.71-3.44 (m, 2H), 1.52 (br s, 1H) ppm.

### Example 5b: 1,4-Di(bromomethyl)naphthaline (building block 2)

### Example 5b.1: 1,4-Naphthalene-dimethanol

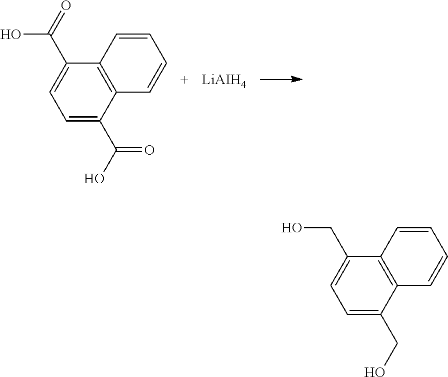

To a mixture of lithium aluminium hydride (12 g, 316 mmol, 3.4 eq.) in THE (350 g) was added portionwise 1,4-naphthalenedicarboxylic acid (20 g, 92.5 mmol, 1 eq.) and the mixture was heated to reflux for 16 hours until TLC (cyclohexane/ethyl acetate 1:1) showed complete conversion.

The mixture was cooled to room temperature and 25 g of water were carefully added. The pH was adjusted to 1.5 by addition of 10% (w/w) aqueous HCl. Ethyl acetate (200 g) and brine (50 g) were added. The phases were separated and the water phase was extracted with ethyl acetate (100 g). The combined organic phases were washed with water (50 mL), saturated aqueous solution of NaHCO₃ (2×50 mL) and saturated aqueous solution of NH₄Cl (50 mL). The organic phase was dried over Na₂SO₄ and concentrated under reduced pressure to yield the titel compound (14.3 g, 76.0 mmol, yield: 82%) as a white solid.

¹H NMR (80 MHz, DMSO-d₆): 5=8.25-7.94 (m, 2H), 7.69-7.36 (m, 4H), 5.36-5.16 (m, 2H), 5.03-4.85 (m, 4H) ppm.

### Example 5b.2:1,4-Di(bromomethyl)naphthalene

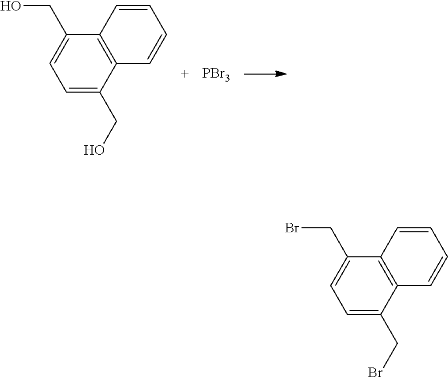

1,4-Naphthalene-dimethanol (14 g, 74 mmol, 1 eq.) obtained in Example 5b.1 above, was dissolved in THE (250 g) at 0° C. Then, phosphortribromide (44.3 g; 163 mmol; 2.2 eq.) was added and the mixture was stirred at room temperature for 24 hours until TLC (cyclohexane/ethyl acetate 1:1) showed complete conversion.

To the mixture was slowly added a saturated aqueous solution of NaHSO₃ (100 mL). To the mixture dichloromethane (400 mL) and water (220 mL) were added. The phases were separated and the water phase was extracted with dichloromethane (100 mL). The combined organic phase was washed with brine and water. The organic phase was concentrated under reduced pressure. To the remaining residue was added methanol (100 g) and the mixture was stirred at room temperature for 2 hours. The title compound 1,4-di(bromomethyl)naphthalene was collected by filtration as a white solid (19.5 g, 62.1 mmol, yield: 83%).

¹H NMR (80 MHz, DMSO-d₆): δ=8.45-8.07 (m, 2H), 7.90-7.48 (m, 4H), 5.21 (s, 4H) ppm.

### Example 5c: 2,2′-[1,4-Naphthylenebis(methyleneoxy[1,1′-binaphthalene]-2′,2-diyloxy)]di(ethan-1-ol) (DBHBNA14 BHN) (compound of formula (Ia), with X=2-hydroxyethyl and A¹=1,4-naphthylene; compound 31 of table A)

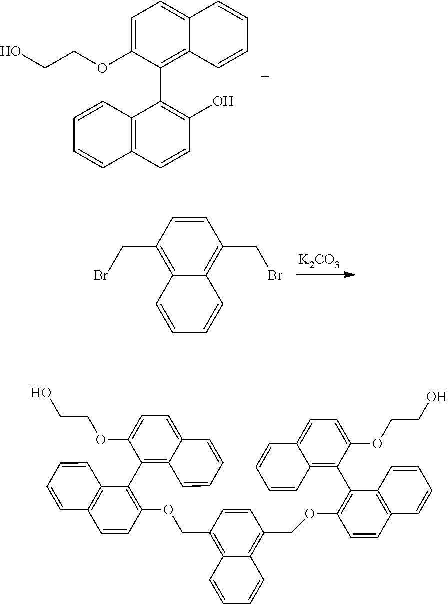

2-[2-Hydroxy-(1,1′-binaphthalene)-2′-oxy]-ethan-1-ol (36.3 g, 110 mmol, 2.1 eq., obtained in Example 5a) was dissolved in acetone (300 g). K₂CO₃ (15.2 g, 110 mmol, 2.1 eq.) was added and the mixture was heated to 60-70° C. Then a solution of 1,4-di(bromomethyl)naphthaline (16.4 g, 52.3 mmol, 1 eq., obtained in Example 5b) in acetone (120 g) was added dropwise. The mixture was stirred under reflux for 1 day until TLC (cyclohexane/ethyl acetate 1:1) showed complete conversion.

To the mixture were added water (300 g) and toluene (300 g). The resulting mixture was concentrated under reduced pressure to remove the acetone and was then acidified by the addition of an aqueous solution of HCl (16% w/w). The aqueous phase was separated and extracted with dichloromethane. The combined organic phases were concentrated under reduced pressure to a thick suspension, which was then stirred for 18 hours at room temperature. The solid product was collected by filtration. The crude product was dissolved in dichloromethane. The resulting solution was washed with water, dried over Na₂SO₄ and concentrated to dryness under reduced pressure to yield the title compound as a white solid (30.0 g, 36.9 mmol, yield: 67%) with a chemical purity of 99.7%.

¹H NMR (80 MHz, CDCl₃): δ=8.05-7.67 (m, 8H), 7.62-6.74 (m, 22H), 5.28 (m_{c}, 4H), 4.07-3.77 (m, 4H), 3.59-3.21 (m, 4H), 2.05 (t, J=5.9 Hz, 2H) ppm.

### Example 6: 2,2′-[1,2-phenylenebis(methyleneoxy[1,1′-binaphthalene]-2′,2-diyloxy)]di(ethan-1-ol) (DBHBNAOBHP) (compound of formula (Ia), with X=2-hydroxyethyl and A¹=1,2-phenylene; compound 26 of Table A)

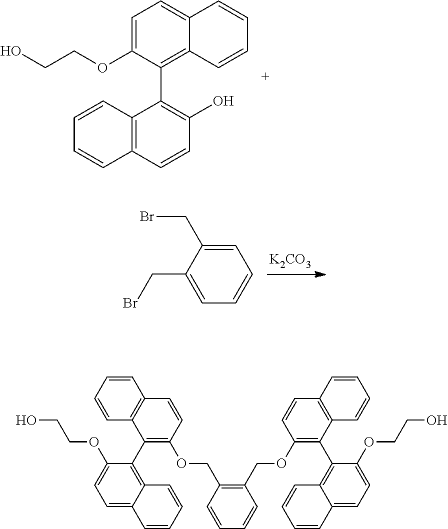

2-[2-Hydroxy-(1,1′-binaphthalene)-2′-oxy]-ethan-1-ol (50 g, 151.3 mmol, 2.1 eq., obtained in Example 5a) was dissolved in acetone (450 g). K₂CO₃ (20.9 g, 151.3 mmol, 2.1 eq.) was added and the mixture was heated to 60-70° C. Then, a solution of 1,2-bis(bromomethyl)benzene (19 g, 72 mmol, 1 eq.) in acetone (180 g) was added dropwise. The mixture was stirred under reflux for 18 hours until TLC (cyclohexane/ethyl acetate 2:1) showed complete conversion.

To the mixture were added water (450 g) and toluene (450 g). The mixture was concentrated under reduced pressure to remove the acetone and was then acidified by the addition of an aqueous solution of HCl (16% w/w). The aqeuous phase was separated and extracted with toluene (225 g). The combined organic phases were washed with water (225 g), dried over Na₂SO₄ and concentrated to dryness under reduced pressure. To the residue was added n-pentane and the mixture was stirred at room temperature for 1 hour. The solid was collected by filtration and dried at 40° C. under vacuum to yield the title compound (42.5 g, 55.7 mmol, yield: 74%) with a chemical purity of 96.7%.

¹H NMR (80 MHz, CDCl₃): δ=8.06-7.65 (m, 8H), 7.53-6.52 (m, 20H), 4.88-4.34 (m, 4H), 4.17-3.79 (m, 4H), 3.62-3.24 (m, 4H), 2.16-1.49 (m, 2H) ppm.

### Example 7: 2,2′-[1,3-phenylenebis(methyleneoxy[1,1′-binaphthalene]-2′,2-diyloxy)]di(ethan-1-ol) (DBHBNAMBHP) (compound of formula (Ia), with X=2-hydroxyethyl and A¹=1,3-phenylene; compound 27 of Table A)

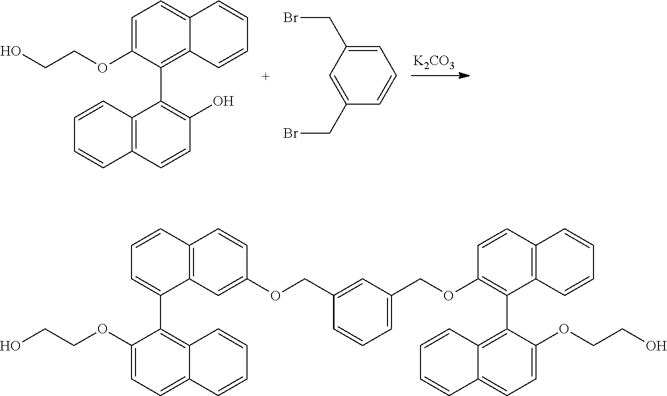

2-[2-Hydroxy-(1,1′-binaphthalene)-2′-oxy]-ethan-1-ol (17.4 g, 52.7 mmol, 2.1 eq., obtained in Example 5a) was dissolved in acetone (150 g). K₂CO₃ (7.3 g, 52.7 mmol, 2.1 eq.) was added and the mixture was heated to 60-70° C. Then a solution of 1,3-bis(bromomethyl)benzene (6.6 g, 25 mmol, 1 eq.) in acetone (60 g) was added dropwise. The mixture was stirred under reflux for 18 hour until TLC (cyclohexane/ethyl acetate 2:1) showed complete conversion.

To the mixture were added water (150 g) and toluene (150 g). The mixture was concentrated under reduced pressure to remove the acetone and was then acidified by the addition of an aqueous solution of HCl (16% w/w). The aqueous phase was separated and extracted with toluene (75 g) at 60° C. The combined organic phases were washed with water (75 g), dried over Na₂SO₄ and concentrated to dryness under reduced pressure. The residue was recrystallized twice from MTBE and dried at 40° C. under vacuum to give the title compound (13.4 g, 17.5 mmol, yield: 66%, chemical purity: 97.8%) as a white solid.

¹H NMR (80 MHz, CDCl₃): δ=8.05-7.69 (m, 8H), 7.52-6.96 (m, 16H), 6.94-6.22 (m, 4H), 4.76 (m_{c}, 4H), 4.19-3.83 (m, 4H), 3.64-3.27 (m, 4H), 1.95 (app. dt, J=6.5, 3.0 Hz, 2H) ppm.

### Example 8a: Dimethyl-2,2′-[1,4-phenylenebis(methyleneoxy[1,1′-binaphthalene]-2′,2-diyloxy)]di(acetic acid ester) (compound of formula (Ia), with X=methoxycarbonylmethyl and A¹=1,4-phenylene; compound 49 of Table A)

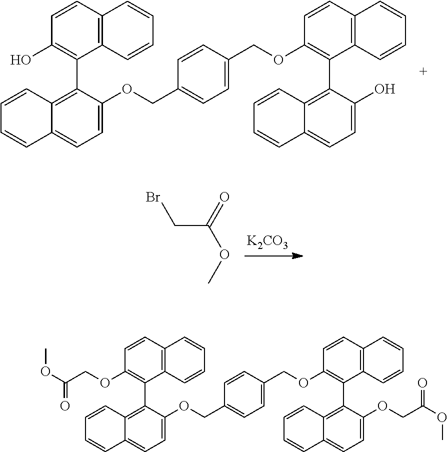

A mixture of 2,2′-[1,4-phenylenebis(methyleneoxy)]di([1,1′-binaphthalen]-2-ol (147 g, 218 mmol, 1.0 eq., obtained in Example 1), K₂CO₃ (90 g, 654 mmol, 3 eq.) and acetone (1500 g) was heated to 50° C. Bromoacetic acid methyl ester (100 g, 654 mmol, 3 eq.) was added dropwise and the reaction mixture was heated under reflux for approximately 12 hours until TLC (cyclohexane/ethyl acetate 2:1) showed complete conversion.

The reaction mixture was concentrated under reduced pressure to remove most of the acetone. Then, water (500 g) and ethyl acetate (1000 g) were added and the mixture was stirred for 30 min. The water phase was separated and extracted with ethyl acetate (250 g). The combined organic phases were washed with brine (500 g), dried over Na₂SO₄ and concentrated to dryness under reduced pressure to give the crude product. n-Pentane was added and the slurry was stirred at room temperature for 1 h. The formed crystals were filtered off to give the desired product as an off-white solid in quantitative yield.

¹H NMR (80 MHz, CDCl₃): δ=8.03-7.72 (m, 8H), 7.48-7.02 (m, 16H), 6.75 (s, 4H), 4.97 (s, 4H), 4.44 (s, 4H), 3.52 (s, 6H) ppm.

### Example 8b: 2,2′-[1,4-phenylenebis(methyleneoxy[1,1′-binaphthalene]-2′,2-diyloxy)]di(acetic acid)

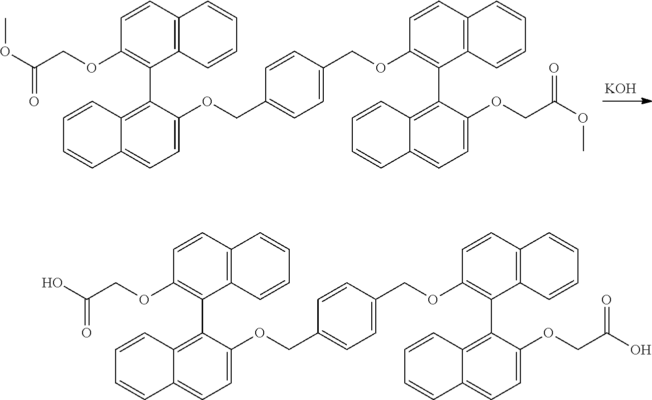

A mixture of dimethyl-2,2′-[1,4-phenylenebis(methyleneoxy[1,1′-binaphthalene]-2′,2-diyloxy)]di(acetic acid ester) (178.4 g, 218 mmol, 1.0 eq., obtained in Example 8a), KOH (133.3 g; 871.6 mmol, 4 eq.), ethanol (424 g) and water (106 g) was heated under reflux for 2 hours. After complete conversion, the mixture was cooled to room temperature and acidified by the addition of diluted aqueous HCl. The water was decanted from the formed solid. Toluene (400 g) was added to the solid and the mixture was heated to reflux for 30 min. The solid was filtered off and the filtrate was concentrated under reduced pressure to give the crude product. This crude product was purified first by stirring in a pentane/MTBE mixture (9:1) for 2 hour and then stirring in MTBE under reflux to give the title compound (84.2 g, 106 mmol, yield: 49%, chemical purity: 99.1%) as a white solid.

¹H NMR (80 MHz, CDCl₃): δ=9.88 (br s, 2H, OH), 8.07-7.53 (m, 8H), 7.48-6.89 (m, 16H), 6.59 (d, J=3.9 Hz, 4H), 5.03-4.49 (m, 4H), 4.45-4.02 (m, 4H) ppm.

### 2.3 Refractive Indices n_{D} of Monomers of Formula (I)

The following table B lists refractive indices of some monomers of formula (I) that were calculated using the software ACD/ChemSketch 2012 (Advanced Chemistry Development, Inc.). The individual monomers are identified in table B by their names and their entry numbers in table A. In addition, it has been verified by quantum chemical calculations for all monomers included in table B that they do not, or only to a negligible extent, absorb in the visible light range and are therefore basically colorless.

TABLE B

| Entry # in | | n_{D} (calc.) |
|---|---|---|
| Table A | monomer name | monomer |
| 1 | 2′,2″-[1,4-phenylenebis(methyleneoxy)]di([1,1′-binaphthalen]- | 1.75 |
| | 2-ol) | |
| 2 | 2′,2″-[1,2-phenylenebis(methyleneoxy)]di([1,1′-binaphthalen]- | 1.75 |
| | 2-ol) | |
| 3 | 2′,2″-[1,3-phenylenebis(methyleneoxy)]di([1,1′-binaphthalen]- | 1.75 |
| | 2-ol) | |
| 14 | 2′,2″-[[1,1′-biphenyl]-4,4′-diylbis(methyleneoxy)]di([1,1′- | 1.74 |
| | binaphthalen]-2-ol) | |
| 13 | 2′,2″-[[1,1′-biphenyl]-3,3′-diylbis(methyleneoxy)]di([1,1′- | 1.74 |
| | binaphthalen]-2-ol) | |
| 4 | 2′,2″-[naphthalene-2,3-diylbis(methyleneoxy)]di([1,1′- | 1.77 |
| | binaphthalen]-2-ol) | |
| 5 | 2′,2″-[naphthalene-2,7-diylbis(methyleneoxy)]di([1,1′- | 1.77 |
| | binaphthalen]-2-ol) | |
| 6 | 2′,2″-[naphthalene-2,6-diylbis(methyleneoxy)]di([1,1′- | 1.77 |
| | binaphthalen]-2-ol) | |
| 7 | 2′,2″-[naphthalene-1,4-diylbis(methyleneoxy)]di([1,1′- | 1.77 |
| | binaphthalen]-2-ol) | |
| 8 | 2′,2″-[naphthalene-1,5-diylbis(methyleneoxy)]di([1,1′- | 1.77 |
| | binaphthalen]-2-ol) | |
| 9 | 2′,2″-[naphthalene-1,8-diylbis(methyleneoxy)]di([1,1′- | 1.77 |
| | binaphthalen]-2-ol) | |
| 10 | 2′,2″-[dibenzo[b,d]thiene-4,6-diylbis(methyleneoxy)]di([1,1′- | 1.79 |
| | binaphthalen]-2-ol) | |
| 11 | 2′,2″-[dibenzo[b,d]thiene-2,8-diylbis(methyleneoxy)]di([1,1′- | 1.79 |
| | binaphthalen]-2-ol) | |
| 17 | 2′,2″-[thianthrene-2,7-diylbis(methyleneoxy)]di([1,1′- | 1.78 |
| | binaphthalen]-2-ol) | |
| 18 | 2′,2″-[thianthrene-2,8-diylbis(methyleneoxy)]di([1,1′- | 1.78 |
| | binaphthalen]-2-ol) | |
| 19 | 2′,2″-[thianthrene-1,4-diylbis(methyleneoxy)]di([1,1′- | 1.78 |
| | binaphthalen]-2-ol) | |
| 20 | 2′,2″-[thianthrene-2,3-diylbis(methyleneoxy)]di([1,1′- | 1.78 |
| | binaphthalen]-2-ol) | |
| 21 | 2′,2″-[thianthrene-1,6-diylbis(methyleneoxy)]di([1,1′- | 1.78 |
| | binaphthalen]-2-ol) | |
| 22 | 2,2′-[thianthrene-1,9-diylbis(methyleneoxy[1,1′-binaphthalene]- | 1.78 |
| | 2′,2-diyloxy)]di(ethan-1-ol) | |
| 15 | 2′,2″-[9H-fluorene-9,9-diylbis(methyleneoxy)]di([1,1′- | 1.75 |
| | binaphthalen]-2-ol) | |
| 23 | 2′,2″-[9H-xanthene-9,9-diylbis(methyleneoxy)]di([1,1′- | 1.74 |
| | binaphthalen]-2-ol) | |
| 24 | 2′,2″-[9H-thioxanthene-9,9-diylbis(methyleneoxy)]di([1,1′- | 1.75 |
| | binaphthalen]-2-ol) | |
| 16 | 2′,2″-[9H-fluorene-2,7-diylbis(methyleneoxy)]di([1,1′- | 1.76 |
| | binaphthalen]-2-ol) | |
| 25 | 2,2′-[1,4-phenylenebis(methyleneoxy[1,1′-binaphthalene]- | 1.71 |
| | 2′,2-diyloxy)]di(ethan-1-ol) | |
| 26 | 2,2′-[1,2-phenylenebis(methyleneoxy[1,1′-binaphthalene]- | 1.71 |
| | 2′,2-diyloxy)]di(ethan-1-ol) | |
| 27 | 2,2′-[1,3-phenylenebis(methyleneoxy[1,1′-binaphthalene]- | 1.71 |
| | 2′,2-diyloxy)]di(ethan-1-ol) | |
| 38 | 2,2′-[[1,1′-biphenyl]-4,4′-diylbis(methyleneoxy[1,1′- | 1.71 |
| | binaphthalene]-2′,2-diyloxy)]di(ethan-1-ol) | |
| 37 | 2,2′-[[1,1′-biphenyl]-3,3′-diylbis(methyleneoxy[1,1′- | 1.71 |
| | binaphthalene]-2′,2-diyloxy)]di(ethan-1-ol) | |
| 28 | 2,2′-[naphthalene-2,3-diylbis(methyleneoxy[1,1′-binaphthalene]- | 1.73 |
| | 2′,2-diyloxy)]di(ethan-1-ol) | |
| 29 | 2,2′-[naphthalene-2,7-diylbis(methyleneoxy[1,1′-binaphthalene]- | 1.73 |
| | 2′,2-diyloxy)]di(ethan-1-ol) | |
| 30 | 2,2′-[naphthalene-2,6-diylbis(methyleneoxy[1,1′-binaphthalene]- | 1.73 |
| | 2′,2-diyloxy)]di(ethan-1-ol) | |
| 31 | 2,2′-[naphthalene-1,4-diylbis(methyleneoxy[1,1′-binaphthalene]- | 1.73 |
| | 2′,2-diyloxy)]di(ethan-1-ol) | |
| 32 | 2,2′-[naphthalene-1,5-diylbis(methyleneoxy[1,1′-binaphthalene]- | 1.73 |
| | 2′,2-diyloxy)]di(ethan-1-ol) | |
| 33 | 2,2′-[naphthalene-1,8-diylbis(methyleneoxy[1,1′-binaphthalene]- | 1.73 |
| | 2′,2-diyloxy)]di(ethan-1-ol) | |
| 34 | 2,2′-[dibenzo[b,d]thiene-4,6-diylbis(methyleneoxy[1,1′- | 1.75 |
| | binaphthalene]-2′,2-diyloxy)]di(ethan-1-ol) | |
| 35 | 2,2′-[dibenzo[b,d]thiene-2,8-diylbis(methyleneoxy[1,1′- | 1.75 |
| | binaphthalene]-2′,2-diyloxy)]di(ethan-1-ol | |
| 41 | 2,2′-[thianthrene-2,7-diylbis(methyleneoxy[1,1′-binaphthalene]- | 1.74 |
| | 2′,2-diyloxy)]di(ethan-1-ol) | |
| 42 | 2,2′-[thianthrene-2,8-diylbis(methyleneoxy[1,1′-binaphthalene]- | 1.74 |
| | 2′,2-diyloxy)]di(ethan-1-ol) | |
| 43 | 2,2′-[thianthrene-1,4-diylbis(methyleneoxy[1,1′-binaphthalene]- | 1.74 |
| | 2′,2-diyloxy)]di(ethan-1-ol) | |
| 44 | 2,2′-[thianthrene-2,3-diylbis(methyleneoxy[1,1′-binaphthalene]- | 1.74 |
| | 2′,2-diyloxy)]di(ethan-1-ol) | |
| 45 | 2,2′-[thianthrene-1,6-diylbis(methyleneoxy[1,1′-binaphthalene]- | 1.74 |
| | 2′,2-diyloxy)]di(ethan-1-ol) | |
| 46 | 2,2′-[thianthrene-1,9-diylbis(methyleneoxy[1,1′-binaphthalene]- | 1.74 |
| | 2′,2-diyloxy)]di(ethan-1-ol) | |
| 39 | 2,2′-[9H-fluorene-9,9-diylbis(methyleneoxy[1,1′-binaphthalene]- | 1.71 |
| | 2′,2-diyloxy)]di(ethan-1-ol) | |
| 47 | 2,2′-[9H-xanthene-9,9-diylbis(methyleneoxy[1,1′-binaphthalene]- | 1.71 |
| | 2′,2-diyloxy)]di(ethan-1-ol) | |
| 48 | 2,2′-[9H-thioxanthene-9,9-diylbis(methyleneoxy[1,1′- | 1.72 |
| | binaphthalene]-2′,2-diyloxy)]di(ethan-1-ol) | |
| 40 | 2,2′-[9H-fluorene-2,7-diylbis(methyleneoxy[1,1′-binaphthalene]- | 1.72 |
| | 2′,2-diyloxy)]di(ethan-1-ol) | |
| 73 | [1,4-phenylenebis(methyleneoxy[1,1′-binaphthalene]-2′,2- | 1.72 |
| | diyloxymethylene-4,1-phenylene)]dimethanol | |
| 97 | [1,4-phenylenebis(methyleneoxy[1,1′-binaphthalene]-2′,2- | 1.72 |
| | diyloxymethylene-3,1-phenylene)]dimethanol | |
| 75 | [1,3-phenylenebis(methyleneoxy[1,1′-binaphthalene]-2′,2- | 1.72 |
| | diyloxymethylene-4,1-phenylene)]dimethanol | |
| 99 | [1,3-phenylenebis(methyleneoxy[1,1′-binaphthalene]-2′,2- | 1.72 |
| | diyloxymethylene-3,1-phenylene)]dimethanol | |
| 86 | [[1,1′-biphenyl]-4,4′-diylbis(methyleneoxy[1,1′-binaphthalene]- | 1.71 |
| | 2′,2-diyloxymethylene-4,1-phenylene)]dimethanol | |
| 110 | [[1,1′-biphenyl]-4,4′-diylbis(methyleneoxy[1,1′-binaphthalene]- | 1.71 |
| | 2′,2-diyloxymethylene-3,1-phenylene)]dimethanol | |
| 85 | [[1,1′-biphenyl]-3,3′-diylbis(methyleneoxy[1,1′-binaphthalene]- | 1.71 |
| | 2′,2-diyloxymethylene-4,1-phenylene)]dimethanol | |
| 109 | [[1,1′-biphenyl]-3,3′-diylbis(methyleneoxy[1,1′-binaphthalene]- | 1.71 |
| | 2′,2-diyloxymethylene-3,1-phenylene)]dimethanol | |
| 77 | [naphthalene-2,7-diylbis(methyleneoxy[1,1′-binaphthalene]- | 1.73 |
| | 2′,2-diyloxymethylene-4,1-phenylene)]dimethanol | |
| 101 | [naphthalene-2,7-diylbis(methyleneoxy[1,1′-binaphthalene]- | 1.73 |
| | 2′,2-diyloxymethylene-3,1-phenylene)]dimethanol | |
| 78 | [naphthalene-2,6-diylbis(methyleneoxy[1,1′-binaphthalene]- | 1.73 |
| | 2′,2-diyloxymethylene-4,1-phenylene)]dimethanol | |
| 102 | [naphthalene-2,6-diylbis(methyleneoxy[1,1′-binaphthalene]- | 1.73 |
| | 2′,2-diyloxymethylene-3,1-phenylene)]dimethanol | |
| 79 | [naphthalene-1,4-diylbis(methyleneoxy[1,1′-binaphthalene]- | 1.73 |
| | 2′,2-diyloxymethylene-4,1-phenylene)]dimethanol | |
| 103 | [naphthalene-1,4-diylbis(methyleneoxy[1,1′-binaphthalene]- | 1.73 |
| | 2′,2-diyloxymethylene-3,1-phenylene)]dimethanol | |
| 80 | [naphthalene-1,5-diylbis(methyleneoxy[1,1′-binaphthalene]- | 1.73 |
| | 2′,2-diyloxymethylene-4,1-phenylene)]dimethanol | |
| 104 | [naphthalene-1,5-diylbis(methyleneoxy[1,1′-binaphthalene]- | 1.73 |
| | 2′,2-diyloxymethylene-3,1-phenylene)]dimethanol | |
| 81 | [naphthalene-1,8-diylbis(methyleneoxy[1,1′-binaphthalene]- | 1.73 |
| | 2′,2-diyloxymethylene-4,1-phenylene)]dimethanol | |
| 105 | [naphthalene-1,8-diylbis(methyleneoxy[1,1′-binaphthalene]- | 1.73 |
| | 2′,2-diyloxymethylene-3,1-phenylene)]dimethanol | |
| 82 | [dibenzo[b,d]thiene-4,6-diylbis(methyleneoxy[1,1′-binaphthalene]- | 1.75 |
| | 2′,2-diyloxymethylene-4,1-phenylene)]dimethanol | |
| 106 | [dibenzo[b,d]thiene-4,6-diylbis(methyleneoxy[1,1′-binaphthalene]- | 1.75 |
| | 2′,2-diyloxymethylene-3,1-phenylene)]dimethanol | |
| 83 | [dibenzo[b,d]thiene-2,8-diylbis(methyleneoxy[1,1′-binaphthalene]- | 1.75 |
| | 2′,2-diyloxymethylene-4,1-phenylene)]dimethanol | |
| 107 | [dibenzo[b,d]thiene-2,8-diylbis(methyleneoxy[1,1′-binaphthalene]- | 1.75 |
| | 2′,2-diyloxymethylene-3,1-phenylene)]dimethanol | |
| 89 | [thianthrene-2,7-diylbis(methyleneoxy[1,1′-binaphthalene]- | 1.74 |
| | 2′,2-diyloxymethylene-4,1-phenylene)]dimethanol | |
| 113 | [thianthrene-2,7-diylbis(methyleneoxy[1,1′-binaphthalene]- | 1.74 |
| | 2′,2-diyloxymethylene-3,1-phenylene)]dimethanol | |
| 90 | [thianthrene-2,8-diylbis(methyleneoxy[1,1′-binaphthalene]- | 1.74 |
| | 2′,2-diyloxymethylene-4,1-phenylene)]dimethanol | |
| 114 | [thianthrene-2,8-diylbis(methyleneoxy[1,1′-binaphthalene]- | 1.74 |
| | 2′,2-diyloxymethylene-3,1-phenylene)]dimethanol | |
| 91 | [thianthrene-1,4-diylbis(methyleneoxy[1,1′-binaphthalene]- | 1.74 |
| | 2′,2-diyloxymethylene-4,1-phenylene)]dimethanol | |
| 115 | [thianthrene-1,4-diylbis(methyleneoxy[1,1′-binaphthalene]- | 1.74 |
| | 2′,2-diyloxymethylene-3,1-phenylene)]dimethanol | |
| 94 | [thianthrene-1,9-diylbis(methyleneoxy[1,1′-binaphthalene]- | 1.74 |
| | 2′,2-diyloxymethylene-4,1-phenylene)]dimethanol | |
| 118 | [thianthrene-1,9-diylbis(methyleneoxy[1,1′-binaphthalene]- | 1.74 |
| | 2′,2-diyloxymethylene-3,1-phenylene)]dimethanol | |
| 87 | [9H-fluorene-9,9-diylbis(methyleneoxy[1,1′-binaphthalene]- | 1.72 |
| | 2′,2-diyloxymethylene-4,1-phenylene)]dimethanol | |
| 111 | [9H-fluorene-9,9-diylbis(methyleneoxy[1,1′-binaphthalene]- | 1.72 |
| | 2′,2-diyloxymethylene-3,1-phenylene)]dimethanol | |
| 95 | [9H-xanthene-9,9-diylbis(methyleneoxy[1,1′-binaphthalene]- | 1.71 |
| | 2′,2-diyloxymethylene-4,1-phenylene)]dimethanol | |
| 119 | [9H-xanthene-9,9-diylbis(methyleneoxy[1,1′-binaphthalene]- | 1.71 |
| | 2′,2-diyloxymethylene-3,1-phenylene)]dimethanol | |
| 96 | [9H-thioxanthene-9,9-diylbis(methyleneoxy[1,1′-binaphthalene]- | 1.72 |
| | 2′,2-diyloxymethylene-4,1-phenylene)]dimethanol | |
| 120 | [9H-thioxanthene-9,9-diylbis(methyleneoxy[1,1′-binaphthalene]- | 1.72 |
| | 2′,2-diyloxymethylene-3,1-phenylene)]dimethanol | |
| 88 | [9H-fluorene-2,7-diylbis(methyleneoxy[1,1′-binaphthalene]- | 1.73 |
| | 2′,2-diyloxymethylene-4,1-phenylene)]dimethanol | |
| 112 | [9H-fluorene-2,7-diylbis(methyleneoxy[1,1′-binaphthalene]- | 1.73 |
| | 2′,2-diyloxymethylene-3,1-phenylene)]dimethanol | |
| 265 | [1,4-phenylenebis(methyleneoxy[1,1′-binaphthalene]-2′,2- | 1.71 |
| | diyloxymethylene[1,1′-biphenyl]-4′,4-diyl)]dimethanol | |
| 266 | 2,2′-[{2,2-bis[(naphthalen-1-yl)methyl]propane-1,3- | 1.72 |
| | diyl}bis(oxy[1,1′-binaphthalene]-2′,2-diyloxy)]di(ethan-1-ol) | |
| — | 2,2′-[methylenebis(oxy[1,1′-binaphthalene]-2′,2- | 1.71 |
| | diyloxy)]di(ethan-1-ol) | |

### 3. Preparation of Thermoplastic Resins from Monomers of Formula (I)

### 3.1 Analytics Relating to Resins Prepared from Monomers of Formula (I)

### Refractive Index (n_{D}):

The refractive index was measured using a disk shaped test piece with a thickness of 3 mm made by polycarbonate resin as a test piece according to JIS B 7071-2:2018. The measurement was conducted at 23° using the refractive index measurement device below.
Refractive index measurement device:
KPR-3000 manufactured by Shimadzu Corporation

### Abbe Number (ν)

A disk shaped test piece with a thickness of 3 mm which is same as the test piece used in the refractive index measurement was used. The refractive index values were measured using the refractive index measurement device below at 23° C. and at wavelengths of 486 nm, 589 nm and 656 nm. Then, the Abbe number was calculated using the below-described formula.
Refractive index measurement device:
KPR-3000 manufactured by Shimadzu Corporation

$$v = ( {n}_{D} - 1 ) / ( {n}_{F} - {n}_{C} )$$

n_{D}: refractive index at a wavelength of 589 nm
n_{c}: refractive index at a wavelength of 656 nm
n_{F}: refractive index at a wavelength of 486 nm

### Glass Transition Temperature (Tg):

The glass transition temperature was measured by differential scanning calorimetry (DSC) using a 10° C./minute heating program according to JIS K7121-1987.
Differential scanning calorimetry device:
X-DSC7000 manufactured by Hitachi High-Tech Science Corporation

### Molecular Weight

The molecular weight distribution of the resin molecules, in particular the values of the weight average molecular weight (Mw) of the resins were measured by the gel permeation chromatography (GPC) method and calculated by the standard polystyrene conversion approach. The following devices, columns and measurement conditions were used:
GPC device: HLC-8420GPC (from Tosoh Corporation);
Columns: three TSKgel SuperHM-M (from Tosoh Corporation),
   one guard column SuperHM-M (from Tosoh Corporation),
   one TSKgel SuperH-RC (from Tosoh Corporation);
Detection Device: RI detection
Standard polystyrene: PstQuick C as standard polystyrene kit (from Tosoh Corporation);
Eluent: tetrahydrofuran;
Flow rate of eluent: 0.6 ml/min;
Column temperature: 40° C.

The number average molecular weight (Mn) values were calculated using similar methods to those used for measuring the Mw values described above. The polystyrene converted weight average molecular weights (Mw) and number average molecular weights (Mn) were calculated using a previously prepared standard curve of polystyrene. Specifically, the standard curve was prepared using a standard polystyrene for which the molecular weight was known (“PStQuick C” from Tosoh Corporation). Further, a calibration curve was obtained by plotting the elution time and molecular weight value of each of the peaks based on the measured data of the standard polystyrene, and conducting three-dimensional approximation. The values for Mw and Mn were calculated based on the following calculation formulae:

$$\begin{matrix}Mw = \sum ( Wi \times Mi ) \div \sum ( W i ) \\ Mn = \sum ( Ni \times Mi ) \div \sum ( W i )\end{matrix}$$

In the calculation formulae, “i” represents the “i”th dividing point, “Wi” represents the molecular weight (g) of the polymer at the “i”th dividing point, “Ni” represents the number of the molecules of the polymer at the “i”th dividing point, and “Mi” represents the molecular mass at the “i”th dividing point. The molecular mass (M) represents the value of the molecular mass of polystyrene at the corresponding elution time in the calibration curve.

### Contents of Low Molecular Weight Compounds (CLWC)

The contents of low molecular weight compounds represent area ratios of compounds with the Mw values lower than 1.000 on GPC analysis. Therefore, contents of low molecular weight compounds were determined according to the following formula:

$$CLWC ( \% ) = \frac{the total area of peaks of compounds with Mw lower than 1. on GPC analysis}{the total area of peaks of compounds on GPC analysis} \times 100$$

The GPC analysis of the low molecular weight compounds is carried out as described above for measuring the molecular weight of the thermoplastic resins.

### Birefringence

The values of Δn (birefringence) were determined as follows: a cast film of 0.1 mm thickness was cut out into a square of 5.0 cm per side, and the film was then clamped at the both ends with chucks (distance between the chucks: 3.0 cm), stretched 1.5-fold at a temperature of the polycarbonate resin's Tg+20° C. and measured for phase difference (Re) at 589 nm using an ellipsometer M-220 (JASCO Corporation, Japan), followed by calculation according to the following equation:

$$Δ n = R e / d$$

Δn: orientation birefringence
Re: phase difference
d: thickness

The criteria of birefringence (Δn) are as shown in the table below.

| Orientation birefringence Δn (×10⁻³) | Evaluation |
|---|---|
| 0 to 1.0 | L (Low) |
| Greater than 1.0 to 10 | M (Medium) |
| Greater than 10 | H (High) |

### 3.2 Examples for the Preparation of Thermoplastic Resins

### 3.2.1 Polycarbonate Resins

### Example 9 (E9)

As materials, 21.6375 g (0.0493 mol) of 9,9-bis[4-(2-hydroxyethoxy)phenyl]fluorene (BPEF), 4.4587 g (0.0055 mol) of 2,2′-[1,4-phenylenebis(methyleneoxy[1,1′-binaphthalene]-2′,2-diyloxy)]di(ethan-1-ol) (DBHBNABHP) that was obtained in Example 2, 12.0983 g (0.0565 mol) of diphenylcarbonate (DPC) and 0.4606×10⁴ g (0.5483×₁₀⁻⁶ mol) of sodium hydrogen carbonate were put into a 300 milliliter reactor with a stirrer and a distillation device. The reactor was flushed with nitrogen and the inside pressure was set to 101.3 kPa.

The reactor was immersed in an oil bath that had been heated to 200° C. and then the ester exchange reaction started. Stirring of the reaction mixture started 5 minutes after the start time of the reaction. 20 minutes later, the pressure of the reaction mixture was reduced from 101.3 kPa to 26.66 kPa over 10 minutes. While the pressure was reducing, the reaction mixture was heated to 210° C. It was then, further heated to 220° C. at the time of 60 minutes after the start time of the reaction. From the time of 80 minutes after the start time of the reaction, the pressure of the reaction mixture was reduced to 20.00 kPa and the reaction mixture was heated to 240° C. in 10 minutes. The pressure of the reaction mixture was then reduced to 0 kPa and maintained at this level for 30 minutes.

Nitrogen gas was introduced into the reactor and, the pressure of the reaction mixture was recovered to 101.3 kPa to finally obtain the polycarbonate resin.

The obtained polycarbonate resin had a refractive index of 1.647, an Abbe number of 22.31, and a Tg of 144° C.; and the polystyrene conversion weight-average molecular weight was 34,459. The molar ratios of the diol monomers used are listed in Table D and the characteristics of the obtained resin are summarized in Table E.

### Example 10 (E10)

The polycarbonate resin was generated according to the same method as used in Example 9 with the only difference that 10.4972 g of DBHBNABHP (0.0138 mol), 14.0818 g of BPEF (0.0321 mol), 10.0725 g of DPC (0.047 mol), and 0.7707×10⁻⁴ g of NaHCO₃ (0.9175×10⁻⁶ mol) were used. The molar ratios of the monomers used are listed in Table D and the characteristics of the obtained resin are summarized in Table E.

### Example 11 (E11)

The polycarbonate resin was generated according to the same method as used in Example 9 with the only difference that 16.7174 g of DBHBNABHP (0.0219 mol), 6.4222 g of BPEF (0.0146 mol), 7.9897 g of DPC (0.0373 mol), and 0.6143×10⁻⁴ g of NaHCO₃ (0.7312×10⁻⁶ mol) were used. The molar ratios of the monomers used are listed in Table D and the characteristics of the obtained resin are summarized in Table E.

Examples 12 to 20, 23 to 27 and 29 to 33 (E12 to E20, E23 to E27 and E29 to E33) The polycarbonate resins of these examples were generated according to the same method as used in Example 9 with the only differences that the amounts and types of the monomers and the catalysts given in Tables C1-C4 were used. The molar ratios of the monomers used in each Example are listed in Table D and the characteristics of the obtained resins are summarized in Table E.

### 3.2.2 Polyester Carbonate Resins

### Example 21 (E21)

As materials, 13.7869 g (0.0181 mol) of 2,2′-[1,4-phenylenebis(methyleneoxy[1,1′-binaphthalene]-2′,2-diyloxy)]di(ethan-1-ol) (DBHBNABHP), 16.2236 g (0.301 mol) of 9,9-bis[6-(2-hydroxyethoxy)2-naphtyl]fluorene (NOLE), 9.5057 g (0.0181 mol) of 6,6′-diphenyl-2,2′bis(2-hydroxyethoxy)-1,1′-binaphtyl (DPBN), 21.8162 g (0.0542 mol) of 2,2′-[(1,1′-binaphthalene-2,2′-diyl)bis(oxy)]diacetic acid (BINOL-DC), 3.0558 g (0.0143 mol) of diphenylcarbonate (DPC) and the catalysts tris(2,4-pentanedionato)aluminium (III) (0.4701×10⁻² g, 0.1449×10⁻³ mol, also termed herein Al(acac)₃) and (4-methylbenzyl)phosphonic acid diethyl ester (0.5882×10⁻² g (0.2428×10⁻³ mol) were put into a 300 milliliter reactor with a stirrer and a distillation device. The reactor was flushed with nitrogen and the inside pressure was set to 101.3 kPa.

The reactor was immersed in an oil bath that had been heated to 200° C. and then the ester exchange reaction started. Stirring of the reaction mixture started 5 minutes after the start time of the reaction. 20 minutes later, the pressure of the reaction mixture was reduced from 101.3 kPa to 93.33 kPa over 10 minutes. From the time of 10 minutes after the pressure reduction was completed, the reaction mixture was heated to 240° C. for 40 minutes. Then, the pressure of the reaction mixture was further reduced to 40.00 kPa over 20 minutes.

Nitrogen gas was introduced into the reactor and the pressure of the reaction mixture was recovered to the normal pressure. Then, the trap was replaced with new one and the reaction conditions were set as 240° C. and 300 Torr. The reaction mixture was heated to 250° C. and the pressure of the reaction mixture was then reduced to 0 kPa in 60 minutes, and maintained at this level for 30 minutes.

Nitrogen gas was introduced into the reactor and, the pressure of the reaction mixture was recovered to 101.3 kPa to finally obtain the polyester carbonate resin.

The obtained polyester carbonate resin had a refractive index of 1.690, an Abbe number of 17.80 and a Tg of 160° C.; and the polystyrene conversion weight-average molecular weight was 36,654. The molar ratios of the monomers used are listed in Table D and the characteristics of the obtained resin are summarized in Table E.

### Example 28 (E28)

The polyester carbonate resin was generated according to the same method as used in Example 21 with the only difference that the amounts and types of the monomers and the catalysts given in Tables C₁-C₄ were used. The molar ratios of the monomers used are listed in Table D and the characteristics of the obtained resin are summarized in Table E.

### 3.2.3 Polyester Resins

### Example 22 (E22)

As materials, 3.5226 g (0.0046 mol) of 2,2′-[1,4-phenylenebis(methyleneoxy[1,1′-binaphthalene]-2′,2-diyloxy)]di(ethan-1-ol) (DBHBNABHP), 2.4287 g (0.0046 mol) of 6,6′-diphenyl-2,2′bis(2-hydroxyethoxy)-1,1′-binaphtyl (DPBN), 1.0031 g (0.0162 mol) of ethylene glycol (EG), 4.6451 g (0.0115 mol) of 2,2′-[(1,1′-binaphthalene-2,2′-diyl)bis(oxy)]diacetic acid (BINOL-DC), 0.1267×10⁻² g (0.5170×10⁻⁵ mol) of manganese(II) acetate tetrahydrate and 0.1251×10⁻² g (0.7100×10⁻⁵ mol) of calcium acetate monohydrate were put into a 50 milliliter reactor with a stirrer and a distillation device. The reactor was flushed with nitrogen.

The reactor was immersed in an oil bath that had been heated to 100° C. and then the ester exchange reaction started. Stirring of the reaction mixture started 5 minutes after the start time of the reaction. By the time of 120 minutes after the start time of the reaction, the reaction mixture was heated to 230° C. and maintained at this level for 290 minutes.

Then, 0.1498×10⁻² g (1.5283×10⁻⁵ mol) of phosphoric acid and 0.4092×10⁻² g (3.9144×10⁻⁵ mol) of germanium dioxide were added to the reaction mixture to start the polycondensation reaction. Then, the reaction mixture was heated to 270° C. and the pressure of the reaction mixture was reduced to 0 kPa in 90 minutes, and maintained at this level for 120 minutes.

Nitrogen gas was introduced into the reactor and the pressure of the reaction mixture was recovered to 101.3 kPa to obtain polyester resin.

The obtained polyester resin had a refractive index of 1.690, an Abbe number of 17.60 and a Tg of 148° C.; and the polystyrene conversion weight-average molecular weight was 34,544. The molar ratios of the monomers used are listed in Table D and the characteristics of the obtained resin are summarized in Table E.

### 3.2.4 Comparative Examples of Polycarbonate Resins

### Comparative Example 1 (CE1)

The polycarbonate resin was generated according to the same method as used in Example 9 with the only difference that 10.2444 g of BNEF (0,019 mol), 19.4593 g of BPEF (0.0444 mol), 13.9877 g of DPC (0.0653 mol), and 0.3838×10⁻⁴ g of NaHCO₃ (0.4568×10⁻⁶ mol) were used. The molar ratios of the monomers used are listed in Table D and the characteristics of the obtained resin are summarized in Table E.

### Comparative Example 2 (CE2)

The polycarbonate resin was generated according to the same method as used in Example 9 with the only difference that 15.2611 g of BNE (0.0408 mol), 11.9149 g of BPEF (0.0272 mol), 14.9882 g of DPC (0.07 mol), and 0.4981×10⁻⁴ g of NaHCO₃ (0.593×10⁻⁶ mol) were used. The molar ratios of the monomers used are listed in Table D and the characteristics of the obtained resin are summarized in Table E.

### Comparative Example 3 (CE3)

The polycarbonate resin was generated according to the same method as used in Example 9 with the only difference that 16.2901 g of BNE (0.0435 mol), 4.7694 g of BPEF (0.0109 mol), 11.8826 g of DPC (0.0555 mol), and 0.4568×10⁻⁴ g of NaHCO₃ (0.5438×10⁻⁶ mol) were used. The molar ratios of the monomers used are listed in Table D and the characteristics of the obtained resin are summarized in Table E.

The following tables C1 to C4 summarize the amounts of the monomers and the catalysts used for preparing the thermoplastic resins of Examples E9 to E33 and CE1 to CE3. The molar ratios of the monomers used in each Example are listed in Table D and the characteristics of the obtained resins are summarized in Table E.
Table C1: Amounts of monomers used in Examples 9 to 33 and CE1 to CE3 (gram base)
Table C2: Amounts of monomers used in Examples 9 to 33 and CE1 to CE3 (mol base)
Table C3: Amounts of catalysts used in Examples 9 to 33 and CE1 to CE3 (gram base)
Table C4: Amounts of catalysts used in Examples 9 to 33 and CE1 to CE3 (mol base)
Table D: Molar ratios of monomers used in Examples 9 to 33 and CE1 to CE3
Table E: Properties of the thermoplastic resins of E9 to E33 and CE1 to CE3

TABLE C1

| Example/ | Monomers | | | | | | |
|---|---|---|---|---|---|---|---|
| Com- | DBHBNA | DMOBBNA | DBNA | DBHBNA14 | DBHBNAM | BNEF | BNE |
| parative | BHP | BHP | BHP | BHN | BHP | (NOLE) | (BHBNA) |
| Example | [g] | [g] | [g] | [g] | [g] | [g] | [g] |
| E9 | 4.4587 | 0.0000 | 0.0000 | 0.0000 | 0.0000 | 0.0000 | 0.0000 |
| E10 | 10.4972 | 0.0000 | 0.0000 | 0.0000 | 0.0000 | 0.0000 | 0.0000 |
| E11 | 16.7174 | 0.0000 | 0.0000 | 0.0000 | 0.0000 | 0.0000 | 0.0000 |
| E12 | 15.4428 | 0.0000 | 0.0000 | 0.0000 | 0.0000 | 0.0000 | 0.0000 |
| E13 | 23.2016 | 0.0000 | 0.0000 | 0.0000 | 0.0000 | 0.0000 | 0.0000 |
| E14 | 28.3503 | 0.0000 | 0.0000 | 0.0000 | 0.0000 | 2.8971 | 0.0000 |
| E15 | 18.5502 | 0.0000 | 0.0000 | 0.0000 | 0.0000 | 18.7101 | 0.0000 |
| E16 | 26.4568 | 0.0000 | 0.0000 | 0.0000 | 0.0000 | 10.0875 | 0.0000 |
| E17 | 21.0494 | 0.0000 | 0.0000 | 0.0000 | 0.0000 | 0.0000 | 0.0000 |
| E18 | 15.5464 | 0.0000 | 0.0000 | 0.0000 | 0.0000 | 5.8541 | 0.0000 |
| E19 | 21.0349 | 0.0000 | 0.0000 | 0.0000 | 0.0000 | 13.2014 | 0.0000 |
| E20 | 5.1868 | 0.0000 | 0.0000 | 0.0000 | 0.0000 | 0.0000 | 0.0000 |
| E21 | 13.7869 | 0.0000 | 0.0000 | 0.0000 | 0.0000 | 16.2236 | 0.0000 |
| E22 | 3.5226 | 0.0000 | 0.0000 | 0.0000 | 0.0000 | 0.0000 | 0.0000 |
| E23 | 0.0000 | 18.3712 | 0.0000 | 0.0000 | 0.0000 | 0.0000 | 0.0000 |
| E24 | 0.0000 | 19.9102 | 0.0000 | 0.0000 | 0.0000 | 0.0000 | 0.0000 |
| E25 | 0.0000 | 29.8562 | 0.0000 | 0.0000 | 0.0000 | 0.0000 | 0.0000 |
| E26 | 0.0000 | 31.1054 | 0.0000 | 0.0000 | 0.0000 | 12.8842 | 0.0000 |
| E27 | 0.0000 | 19.9877 | 0.0000 | 0.0000 | 0.0000 | 0.0000 | 0.0000 |
| E28 | 0.0000 | 15.2398 | 0.0000 | 0.0000 | 0.0000 | 11.9605 | 0.0000 |
| E29 | 0.0000 | 0.0000 | 6.8655 | 0.0000 | 0.0000 | 0.0000 | 0.0000 |
| E30 | 22.0322 | 0.0000 | 12.0244 | 0.0000 | 0.0000 | 0.0000 | 0.0000 |
| E31 | 18.0343 | 0.0000 | 16.8901 | 0.0000 | 0.0000 | 0.0000 | 0.0000 |
| E32 | 0.0000 | 0.0000 | 0.0000 | 11.1521 | 0.0000 | 0.0000 | 0.0000 |
| E33 | 0.0000 | 0.0000 | 0.0000 | 0.0000 | 12.6169 | 0.0000 | 0.0000 |
| CE1 | 0.0000 | 0.0000 | 0.0000 | 0.0000 | 0.0000 | 10.2444 | 0.0000 |
| CE2 | 0.0000 | 0.0000 | 0.0000 | 0.0000 | 0.0000 | 0.0000 | 15.2611 |
| CE3 | 0.0000 | 0.0000 | 0.0000 | 0.0000 | 0.0000 | 0.0000 | 4.7694 |

| Example/ | Monomers | | | | | |
|---|---|---|---|---|---|---|
| Com- | DPBHBNA | BPPEF | | | | |
| parative | (DPBN) | (PG) | BPEF | EG | BINOL-DC | DPC |
| Example | [g] | [g] | [g] | [g] | [g] | [g] |
| E9 | 0.0000 | 0.0000 | 21.6375 | 0.0000 | 0.0000 | 12.0983 |
| E10 | 0.0000 | 0.0000 | 14.0818 | 0.0000 | 0.0000 | 10.0725 |
| E11 | 0.0000 | 0.0000 | 6.4222 | 0.0000 | 0.0000 | 7.9897 |
| E12 | 0.0000 | 9.5658 | 13.6105 | 0.0000 | 0.0000 | 14.8879 |
| E13 | 0.0000 | 19.4620 | 0.0000 | 0.0000 | 0.0000 | 13.9800 |
| E14 | 0.0000 | 3.7549 | 0.0000 | 0.0000 | 0.0000 | 10.7888 |
| E15 | 0.0000 | 6.1552 | 0.0000 | 0.0000 | 0.0000 | 15.0310 |
| E16 | 0.0000 | 9.4227 | 0.0000 | 0.0000 | 0.0000 | 15.0067 |
| E17 | 12.3361 | 10.5940 | 0.0000 | 0.0000 | 0.0000 | 15.2198 |
| E18 | 19.2939 | 0.0000 | 0.0000 | 0.0000 | 0.0000 | 14.9878 |
| E19 | 4.8343 | 0.0000 | 0.0000 | 0.0000 | 0.0000 | 13.1918 |
| E20 | 27.8939 | 4.8193 | 0.0000 | 0.0000 | 0.0000 | 15.0012 |
| E21 | 9.5057 | 0.0000 | 0.0000 | 0.0000 | 21.8162 | 3.0558 |
| E22 | 2.4287 | 0.0000 | 0.0000 | 1.0031 | 4.6451 | 0.0000 |
| E23 | 0.0000 | 0.0000 | 20.5412 | 0.0000 | 0.0000 | 14.7654 |
| E24 | 0.0000 | 7.2830 | 16.8555 | 0.0000 | 0.0000 | 16.0023 |
| E25 | 0.0000 | 20.8786 | 0.0000 | 0.0000 | 0.0000 | 14.9976 |
| E26 | 0.0000 | 2.9747 | 0.0000 | 0.0000 | 0.0000 | 13.8890 |
| E27 | 24.4140 | 0.0000 | 0.0000 | 0.0000 | 0.0000 | 15.0606 |
| E28 | 11.6798 | 0.0000 | 0.0000 | 0.0000 | 20.1043 | 2.8160 |
| E29 | 0.0000 | 0.0000 | 17.8458 | 0.0000 | 0.0000 | 11.2244 |
| E30 | 0.0000 | 8.7112 | 0.0000 | 0.0000 | 0.0000 | 13.5578 |
| E31 | 10.9714 | 0.0000 | 0.0000 | 0.0000 | 0.0000 | 15.3409 |
| E32 | 0.0000 | 0.0000 | 24.0622 | 0.0000 | 0.0000 | 14.9874 |
| E33 | 0.0000 | 0.0000 | 16.9213 | 0.0000 | 0.0000 | 12.0451 |
| CE1 | 0.0000 | 0.0000 | 19.4593 | 0.0000 | 0.0000 | 13.9877 |
| CE2 | 0.0000 | 0.0000 | 11.9149 | 0.0000 | 0.0000 | 14.9882 |
| CE3 | 0.0000 | 0.0000 | 16.2901 | 0.0000 | 0.0000 | 11.8826 |

TABLE C2

| Exam- | | | | | | | | | | | | | |
|---|---|---|---|---|---|---|---|---|---|---|---|---|---|
| ple/ | | | | | | | | | | | | | |
| Coma- | Monomers | | | | | | | | | | | | |
| partive | DBHBNA | DMOBBNA | DBNA | DBHBNA14 | DBHBNAM | | | | | | | BINOL- | |
| Exam- | BHP | BHP | BHP | BHN | BHP | NOLE | BNE | DPBN | PG | BPEF | EG | DC | DPC |
| ple | [mol] | [mol] | [mol] | 0.0000 | 0.0000 | [mol] | [mol] | [mol] | [mol] | [mol] | [mol] | [mol] | [mol] |
| E9 | 0.0055 | 0.0000 | 0.0000 | 0.0000 | 0.0000 | 0.0000 | 0.0000 | 0.0000 | 0.0000 | 0.0493 | 0.0000 | 0.0000 | 0.0565 |
| E10 | 0.0138 | 0.0000 | 0.0000 | 0.0000 | 0.0000 | 0.0000 | 0.0000 | 0.0000 | 0.0000 | 0.0321 | 0.0000 | 0.0000 | 0.0470 |
| E11 | 0.0219 | 0.0000 | 0.0000 | 0.0000 | 0.0000 | 0.0000 | 0.0000 | 0.0000 | 0.0000 | 0.0146 | 0.0000 | 0.0000 | 0.0373 |
| E12 | 0.0202 | 0.0000 | 0.0000 | 0.0000 | 0.0000 | 0.0000 | 0.0000 | 0.0000 | 0.0162 | 0.0310 | 0.0000 | 0.0000 | 0.6747 |
| E13 | 0.0304 | 0.0000 | 0.0000 | 0.0000 | 0.0000 | 0.0000 | 0.0000 | 0.0000 | 0.0329 | 0.0000 | 0.0000 | 0.0000 | 0.0695 |
| E14 | 0.0372 | 0.0000 | 0.0000 | 0.0000 | 0.0000 | 0.0054 | 0.0000 | 0.0000 | 0.0064 | 0.0000 | 0.0000 | 0.0000 | 0.0504 |
| E15 | 0.0243 | 0.0000 | 0.0000 | 0.0000 | 0.0000 | 0.0349 | 0.0000 | 0.0000 | 0.0105 | 0.0000 | 0.0000 | 0.0000 | 0.0702 |
| E16 | 0.0347 | 0.0000 | 0.0000 | 0.0000 | 0.0000 | 0.0188 | 0.0000 | 0.0000 | 0.0161 | 0.0000 | 0.0000 | 0.0000 | 0.0701 |
| E17 | 0.0276 | 0.0000 | 0.0000 | 0.0000 | 0.0000 | 0.0000 | 0.0000 | 0.0235 | 0.0179 | 0.0000 | 0.0000 | 0.0000 | 0.0710 |
| E18 | 0.0204 | 0.0000 | 0.0000 | 0.0000 | 0.0000 | 0.0109 | 0.0000 | 0.0367 | 0.0000 | 0.0000 | 0.0000 | 0.0000 | 0.0700 |
| E19 | 0.0276 | 0.0000 | 0.0000 | 0.0000 | 0.0000 | 0.8795 | 0.0000 | 0.0092 | 0.0000 | 0.0000 | 0.0000 | 0.0000 | 0.0000 |
| E20 | 0.0068 | 0.0000 | 0.0000 | 0.0000 | 0.0000 | 0.0000 | 0.0000 | 0.0530 | 0.0082 | 0.0000 | 0.0000 | 0.0000 | 0.0700 |
| E21 | 0.0181 | 0.0000 | 0.0000 | 0.0000 | 0.0000 | 0.0301 | 0.0000 | 0.0181 | 0.0000 | 0.0000 | 0.0000 | 0.0542 | 0.0143 |
| E22 | 0.0046 | 0.0000 | 0.0000 | 0.0000 | 0.0000 | 0.0000 | 0.0000 | 0.0046 | 0.0000 | 0.0000 | 0.0162 | 0.0115 | 0.0000 |
| E23 | 0.0000 | 0.0201 | 0.0000 | 0.0000 | 0.0000 | 0.0000 | 0.0000 | 0.0000 | 0.0000 | 0.0468 | 0.0000 | 0.0000 | 0.0689 |
| E24 | 0.0000 | 0.0218 | 0.0000 | 0.0000 | 0.0000 | 0.0000 | 0.0000 | 0.0000 | 0.0123 | 0.0384 | 0.0000 | 0.0000 | 0.0747 |
| E25 | 0.0000 | 0.0326 | 0.0000 | 0.0000 | 0.0000 | 0.0000 | 0.0000 | 0.0000 | 0.0353 | 0.0000 | 0.0000 | 0.0000 | 0.0700 |
| E26 | 0.0000 | 0.0340 | 0.0000 | 0.0000 | 0.0000 | 0.0239 | 0.0000 | 0.0000 | 0.0050 | 0.0000 | 0.0000 | 0.0000 | 0.0648 |
| E27 | 0.0000 | 0.0218 | 0.0000 | 0.0000 | 0.0000 | 0.0000 | 0.0000 | 0.0464 | 0.0000 | 0.0000 | 0.0000 | 0.0000 | 0.0703 |
| E28 | 0.0000 | 0.0167 | 0.0000 | 0.0000 | 0.0000 | 0.0222 | 0.0000 | 0.0222 | 0.0000 | 0.0000 | 0.0000 | 0.0500 | 0.0131 |
| E29 | 0.0000 | 0.0000 | 0.0102 | 0.0000 | 0.0000 | 0.0000 | 0.0000 | 0.0000 | 0.0000 | 0.0407 | 0.0000 | 0.0000 | 0.0524 |
| E30 | 0.0289 | 0.0000 | 0.0178 | 0.0000 | 0.0000 | 0.0000 | 0.0000 | 0.0000 | 0.0147 | 0.0000 | 0.0000 | 0.0000 | 0.0633 |
| E31 | 0.0236 | 0.0000 | 0.0250 | 0.0000 | 0.0000 | 0.0000 | 0.0000 | 0.0209 | 0.0000 | 0.0000 | 0.0000 | 0.0000 | 0.0716 |
| E32 | 0.0000 | 0.0000 | 0.0000 | 0.0137 | 0.0000 | 0.0000 | 0.0000 | 0.0000 | 0.0000 | 0.0549 | 0.0000 | 0.0000 | 0.0700 |
| E33 | 0.0000 | 0.0000 | 0.0000 | 0.0000 | 0.0165 | 0.0000 | 0.0000 | 0.0000 | 0.0000 | 0.0386 | 0.0000 | 0.0000 | 0.0562 |
| CE1 | 0.0000 | 0.0000 | 0.0000 | 0.0000 | 0.0000 | 0.0190 | 0.0000 | 0.0000 | 0.0000 | 0.0444 | 0.0000 | 0.0000 | 0.0653 |
| CE2 | 0.0000 | 0.0000 | 0.0000 | 0.0000 | 0.0000 | 0.0000 | 0.0408 | 0.0000 | 0.0000 | 0.0272 | 0.0000 | 0.0000 | 0.0700 |
| CE3 | 0.0000 | 0.0000 | 0.0000 | 0.0000 | 0.0000 | 0.0000 | 0.0435 | 0.0000 | 0.0000 | 0.0109 | 0.0000 | 0.0000 | 0.0555 |

TABLE C3

| | Catalysts | | | | | | |
|---|---|---|---|---|---|---|---|
| Example/ | | | Diethyl (4- | Mn(OAc)₂• | Ca(OAc)₂• | | |
| Comparative | NaHCO₃ | Al(acac)₃ | Methylbenzyl)phosphonate | 4H₂O | H₂O | H₃PO₄ | GeO₂ |
| Example | [× 10⁻⁴ g] | [× 10⁻² g] | [× 10⁻² g] | [× 10⁻² g] | [× 10⁻² g] | [× 10⁻² g] | [× 10⁻² g] |
| E9 | 0.9212 | 0.0000 | 0.0000 | 0.0000 | 0.0000 | 0.0000 | 0.0000 |
| E10 | 0.7707 | 0.0000 | 0.0000 | 0.0000 | 0.0000 | 0.0000 | 0.0000 |
| E11 | 0.6143 | 0.0000 | 0.0000 | 0.0000 | 0.0000 | 0.0000 | 0.0000 |
| E12 | 0.5668 | 0.0000 | 0.0000 | 0.0000 | 0.0000 | 0.0000 | 0.0000 |
| E13 | 0.5323 | 0.0000 | 0.0000 | 0.0000 | 0.0000 | 0.0000 | 0.0000 |
| E14 | 0.4108 | 0.0000 | 0.0000 | 0.0000 | 0.0000 | 0.0000 | 0.0000 |
| E15 | 0.5836 | 0.0000 | 0.0000 | 0.0000 | 0.0000 | 0.0000 | 0.0000 |
| E16 | 0.5827 | 0.0000 | 0.0000 | 0.0000 | 0.0000 | 0.0000 | 0.0000 |
| E17 | 0.5795 | 0.0000 | 0.0000 | 0.0000 | 0.0000 | 0.0000 | 0.0000 |
| E18 | 0.5707 | 0.0000 | 0.0000 | 0.0000 | 0.0000 | 0.0000 | 0.0000 |
| E19 | 0.5148 | 0.0000 | 0.0000 | 0.0000 | 0.0000 | 0.0000 | 0.0000 |
| E20 | 0.5712 | 0.0000 | 0.0000 | 0.0000 | 0.0000 | 0.0000 | 0.0000 |
| E21 | 0.0000 | 0.4701 | 0.5882 | 0.0000 | 0.0000 | 0.0000 | 0.0000 |
| E22 | 0.0000 | 0.0000 | 0.0000 | 0.1267 | 0.1251 | 0.1498 | 0.4092 |
| E23 | 0.5622 | 0.0000 | 0.0000 | 0.0000 | 0.0000 | 0.0000 | 0.0000 |
| E24 | 0.6093 | 0.0000 | 0.0000 | 0.0000 | 0.0000 | 0.0000 | 0.0000 |
| E25 | 0.5710 | 0.0000 | 0.0000 | 0.0000 | 0.0000 | 0.0000 | 0.0000 |
| E26 | 0.5288 | 0.0000 | 0.0000 | 0.0000 | 0.0000 | 0.0000 | 0.0000 |
| E27 | 0.5734 | 0.0000 | 0.0000 | 0.0000 | 0.0000 | 0.0000 | 0.0000 |
| E28 | 0.0000 | 0.4525 | 0.5662 | 0.0000 | 0.0000 | 0.0000 | 0.0000 |
| E29 | 0.4274 | 0.0000 | 0.0000 | 0.0000 | 0.0000 | 0.0000 | 0.0000 |
| E30 | 0.5162 | 0.0000 | 0.0000 | 0.0000 | 0.0000 | 0.0000 | 0.0000 |
| E31 | 0.5841 | 0.0000 | 0.0000 | 0.0000 | 0.0000 | 0.0000 | 0.0000 |
| E32 | 9.7959 | 0.0000 | 0.0000 | 0.0000 | 0.0000 | 0.0000 | 0.0000 |
| E33 | 3.7049 | 0.0000 | 0.0000 | 0.0000 | 0.0000 | 0.0000 | 0.0000 |
| CE1 | 0.3838 | 0.0000 | 0.0000 | 0.0000 | 0.0000 | 0.0000 | 0.0000 |
| CE2 | 0.4981 | 0.0000 | 0.0000 | 0.0000 | 0.0000 | 0.0000 | 0.0000 |
| CE3 | 0.4568 | 0.0000 | 0.0000 | 0.0000 | 0.0000 | 0.0000 | 0.0000 |

TABLE C4

| | Catalysts | | | | | | |
|---|---|---|---|---|---|---|---|
| Example/ | | | Diethyl (4- | Mn(OAc)₂• | Ca(OAc)₂• | | |
| Comparative | NaHCO₃ | Al(acac)₃ | Methylbenzyl)phosphonate | 4H₂O | H₂O | H₃PO₄ | GeO₂ |
| Example | [× 10⁻⁶ mol] | [× 10⁻³ mol] | [× 10⁻³ mol] | [× 10⁻⁵ mol] | [× 10⁻⁵ mol] | [× 10⁻⁵ mol] | [× 10⁻⁵ mol] |
| E9 | 0.5483 | 0.0000 | 0.0000 | 0.0000 | 0.0000 | 0.0000 | 0.0000 |
| E10 | 0.9175 | 0.0000 | 0.0000 | 0.0000 | 0.0000 | 0.0000 | 0.0000 |
| E11 | 0.7312 | 0.0000 | 0.0000 | 0.0000 | 0.0000 | 0.0000 | 0.0000 |
| E12 | 0.6747 | 0.0000 | 0.0000 | 0.0000 | 0.0000 | 0.0000 | 0.0000 |
| E13 | 0.6336 | 0.0000 | 0.0000 | 0.0000 | 0.0000 | 0.0000 | 0.0000 |
| E14 | 0.4890 | 0.0000 | 0.0000 | 0.0000 | 0.0000 | 0.0000 | 0.0000 |
| E15 | 0.6947 | 0.0000 | 0.0000 | 0.0000 | 0.0000 | 0.0000 | 0.0000 |
| E16 | 0.6936 | 0.0000 | 0.0000 | 0.0000 | 0.0000 | 0.0000 | 0.0000 |
| E17 | 0.6898 | 0.0000 | 0.0000 | 0.0000 | 0.0000 | 0.0000 | 0.0000 |
| E18 | 0.6793 | 0.0000 | 0.0000 | 0.0000 | 0.0000 | 0.0000 | 0.0000 |
| E19 | 0.6128 | 0.0000 | 0.0000 | 0.0000 | 0.0000 | 0.0000 | 0.0000 |
| E20 | 0.6799 | 0.0000 | 0.0000 | 0.0000 | 0.0000 | 0.0000 | 0.0000 |
| E21 | 0.0000 | 0.1449 | 0.2428 | 0.0000 | 0.0000 | 0.0000 | 0.0000 |
| E22 | 0.0000 | 0.0000 | 0.0000 | 0.5170 | 0.7100 | 1.5283 | 3.9114 |
| E23 | 0.6692 | 0.0000 | 0.0000 | 0.0000 | 0.0000 | 0.0000 | 0.0000 |
| E24 | 0.7252 | 0.0000 | 0.0000 | 0.0000 | 0.0000 | 0.0000 | 0.0000 |
| E25 | 0.6797 | 0.0000 | 0.0000 | 0.0000 | 0.0000 | 0.0000 | 0.0000 |
| E26 | 0.6295 | 0.0000 | 0.0000 | 0.0000 | 0.0000 | 0.0000 | 0.0000 |
| E27 | 0.6826 | 0.0000 | 0.0000 | 0.0000 | 0.0000 | 0.0000 | 0.0000 |
| E28 | 0.0000 | 0.1395 | 0.2337 | 0.0000 | 0.0000 | 0.0000 | 0.0000 |
| E29 | 0.5087 | 0.0000 | 0.0000 | 0.0000 | 0.0000 | 0.0000 | 0.0000 |
| E30 | 0.6145 | 0.0000 | 0.0000 | 0.0000 | 0.0000 | 0.0000 | 0.0000 |
| E31 | 0.6953 | 0.0000 | 0.0000 | 0.0000 | 0.0000 | 0.0000 | 0.0000 |
| E32 | 11.6604 | 0.0000 | 0.0000 | 0.0000 | 0.0000 | 0.0000 | 0.0000 |
| E33 | 4.4101 | 0.0000 | 0.0000 | 0.0000 | 0.0000 | 0.0000 | 0.0000 |
| CE1 | 0.4568 | 0.0000 | 0.0000 | 0.0000 | 0.0000 | 0.0000 | 0.0000 |
| CE2 | 0.5930 | 0.0000 | 0.0000 | 0.0000 | 0.0000 | 0.0000 | 0.0000 |
| CE3 | 0.5438 | 0.0000 | 0.0000 | 0.0000 | 0.0000 | 0.0000 | 0.0000 |

TABLE D

| Exam- | | | | | | | | | | | | | |
|---|---|---|---|---|---|---|---|---|---|---|---|---|---|
| ple/ | Monomers | | | | | | | | | | | | |
| Coma- | | | | | | | | | BPPEF | | | | Type |
| partive | DBHBNA | DMOBBNA | DBNA | DBHBNA14 | DBHBNAM | BNEF | BNE | DPBHBNA | (PG) | BPEF | EG | BINOL- | of |
| Exam- | BHP | BHP | BHP | BHN | BHP | (NOLE) | (BHBNA) | (DPBN) | [mol | [mol | [mol | DC | Resin |
| ple | [mol %] | [mol %] | [mol %] | [mol %] | [mol %] | [mol %] | [mol %] | [mol %] | %] | %] | %] | [mol %] | * |
| E9 | 10 | 0 | 0 | 0 | 0 | 0 | 0 | 0 | 0 | 90 | 0 | 0 | PC |
| E10 | 30 | 0 | 0 | 0 | 0 | 0 | 0 | 0 | 0 | 70 | 0 | 0 | PC |
| E11 | 60 | 0 | 0 | 0 | 0 | 0 | 0 | 0 | 0 | 40 | 0 | 0 | PC |
| E12 | 30 | 0 | 0 | 0 | 0 | 0 | 0 | 0 | 24 | 46 | 0 | 0 | PC |
| E13 | 48 | 0 | 0 | 0 | 0 | 0 | 0 | 0 | 52 | 0 | 0 | 0 | PC |
| E14 | 76 | 0 | 0 | 0 | 0 | 11 | 0 | 0 | 13 | 0 | 0 | 0 | PC |
| E15 | 35 | 0 | 0 | 0 | 0 | 50 | 0 | 0 | 15 | 0 | 0 | 0 | PC |
| E16 | 50 | 0 | 0 | 0 | 0 | 27 | 0 | 0 | 23 | 0 | 0 | 0 | PC |
| E17 | 40 | 0 | 0 | 0 | 0 | 0 | 0 | 34 | 26 | 0 | 0 | 0 | PC |
| E18 | 30 | 0 | 0 | 0 | 0 | 16 | 0 | 54 | 0 | 0 | 0 | 0 | PC |
| E19 | 45 | 0 | 0 | 0 | 0 | 40 | 0 | 15 | 0 | 0 | 0 | 0 | PC |
| E20 | 10 | 0 | 0 | 0 | 0 | 0 | 0 | 78 | 12 | 0 | 0 | 0 | PC |
| E21 | 15 | 0 | 0 | 0 | 0 | 25 | 0 | 15 | 0 | 0 | 0 | 45 | PEC |
| E22 | 20 | 0 | 0 | 0 | 0 | 0 | 0 | 20 | 0 | 0 | 10 | 50 | PE |
| E23 | 0 | 30 | 0 | 0 | 0 | 0 | 0 | 0 | 0 | 70 | 0 | 0 | PC |
| E24 | 0 | 30 | 0 | 0 | 0 | 0 | 0 | 0 | 17 | 53 | 0 | 0 | PC |
| E25 | 0 | 48 | 0 | 0 | 0 | 0 | 0 | 0 | 52 | 0 | 0 | 0 | PC |
| E26 | 0 | 54 | 0 | 0 | 0 | 38 | 0 | 0 | 8 | 0 | 0 | 0 | PC |
| E27 | 0 | 32 | 0 | 0 | 0 | 0 | 0 | 68 | 0 | 0 | 0 | 0 | PC |
| E28 | 0 | 15 | 0 | 0 | 0 | 20 | 0 | 20 | 0 | 0 | 0 | 45 | PEC |
| E29 | 0 | 0 | 20 | 0 | 0 | 0 | 0 | 0 | 0 | 80 | 0 | 0 | PC |
| E30 | 47 | 0 | 29 | 0 | 0 | 0 | 0 | 0 | 24 | 0 | 0 | 0 | PC |
| E31 | 34 | 0 | 36 | 0 | 0 | 0 | 0 | 30 | 0 | 0 | 0 | 0 | PC |
| E32 | 0 | 0 | 0 | 20 | 0 | 0 | 0 | 0 | 0 | 80 | 0 | 0 | PC |
| E33 | 0 | 0 | 0 | 0 | 30 | 0 | 0 | 0 | 0 | 70 | 0 | 0 | PC |
| CE1 | 0 | 0 | 0 | 0 | 0 | 30 | 0 | 0 | 0 | 70 | 0 | 0 | PC |
| CE2 | 0 | 0 | 0 | 0 | 0 | 0 | 60 | 0 | 0 | 40 | 0 | 0 | PC |
| CE3 | 0 | 0 | 0 | 0 | 0 | 0 | 80 | 0 | 0 | 20 | 0 | 0 | PC |

*) PC: polycarbonate; PEC: polyester carbonate; PE: polyester

TABLE E

| Example/ | Characteristics | | | | | | |
|---|---|---|---|---|---|---|---|
| Coma- | | | | | | | |
| partive | n_{D} | v | Tg | | | CLWC | Bire- |
| Example | [—] | [—] | [° C.] | Mw | Mn | [%] | fringence |
| E9 | 1.647 | 22.31 | 144 | 26999 | 13987 | 1.9 | L |
| E10 | 1.658 | 20.92 | 140 | 26643 | 13563 | 3.1 | L |
| E11 | 1.672 | 19.17 | 136 | 28883 | 15002 | 5.0 | M |
| E12 | 1.660 | 20.72 | 143 | 31292 | 16673 | 3.3 | L |
| E13 | 1.670 | 19.63 | 144 | 32209 | 16509 | 4.7 | L |
| E14 | 1.680 | 18.55 | 140 | 27236 | 14098 | 5.4 | H |
| E15 | 1.6801 | 18.80 | 150.2 | 19005 | 10204 | 3.4 | M |
| E16 | 1.6789 | 18.87 | 150.6 | 25710 | 13406 | 3.7 | M |
| E17 | 1.680 | 18.12 | 147 | 30981 | 15926 | 5.9 | L |
| E18 | 1.690 | 17.00 | 155 | 34088 | 18884 | 6.4 | H |
| E19 | 1.6854 | 18.05 | 148 | 22262 | 13056 | 4.8 | H |
| E20 | 1.690 | 16.60 | 158 | 28779 | 15494 | 6.6 | M |
| E21 | 1.690 | 17.80 | 160 | 36654 | 18898 | 5.1 | H |
| E22 | 1.690 | 17.60 | 148 | 34544 | 18041 | 4.8 | M |
| E23 | 1.658 | 21.08 | 142 | 36043 | 18560 | 1.7 | L |
| E24 | 1.660 | 20.97 | 145 | 32108 | 17221 | 1.1 | L |
| E25 | 1.670 | 19.95 | 148 | 29800 | 15980 | 1.6 | L |
| E26 | 1.680 | 19.00 | 156 | 28992 | 15348 | 2.1 | H |
| E27 | 1.690 | 17.03 | 153 | 27802 | 14908 | 5.4 | M |
| E28 | 1.690 | 17.81 | 160 | 34788 | 18121 | 4.2 | H |
| E29 | 1.654 | 21.48 | 155 | 27770 | 14560 | 1.6 | L |
| E30 | 1.680 | 18.60 | 150 | 32242 | 17943 | 3.9 | L |
| E31 | 1.690 | 17.30 | 157 | 28876 | 15015 | 5.8 | M |
| E32 | 1.6588 | 21.09 | 138.8 | 11727 | 8631 | 3.4 | L |
| E33 | 1.6590 | 20.90 | 137.2 | 66553 | 33602 | 3.5 | L |
| CE1 | 1.655 | 21.56 | 160 | 31367 | 16246 | 1.0 | H |
| CE2 | 1.655 | 20.66 | 128 | 25631 | 14306 | 3.6 | L |
| CE3 | 1.661 | 19.78 | 121 | 28457 | 15398 | 7.5 | L |

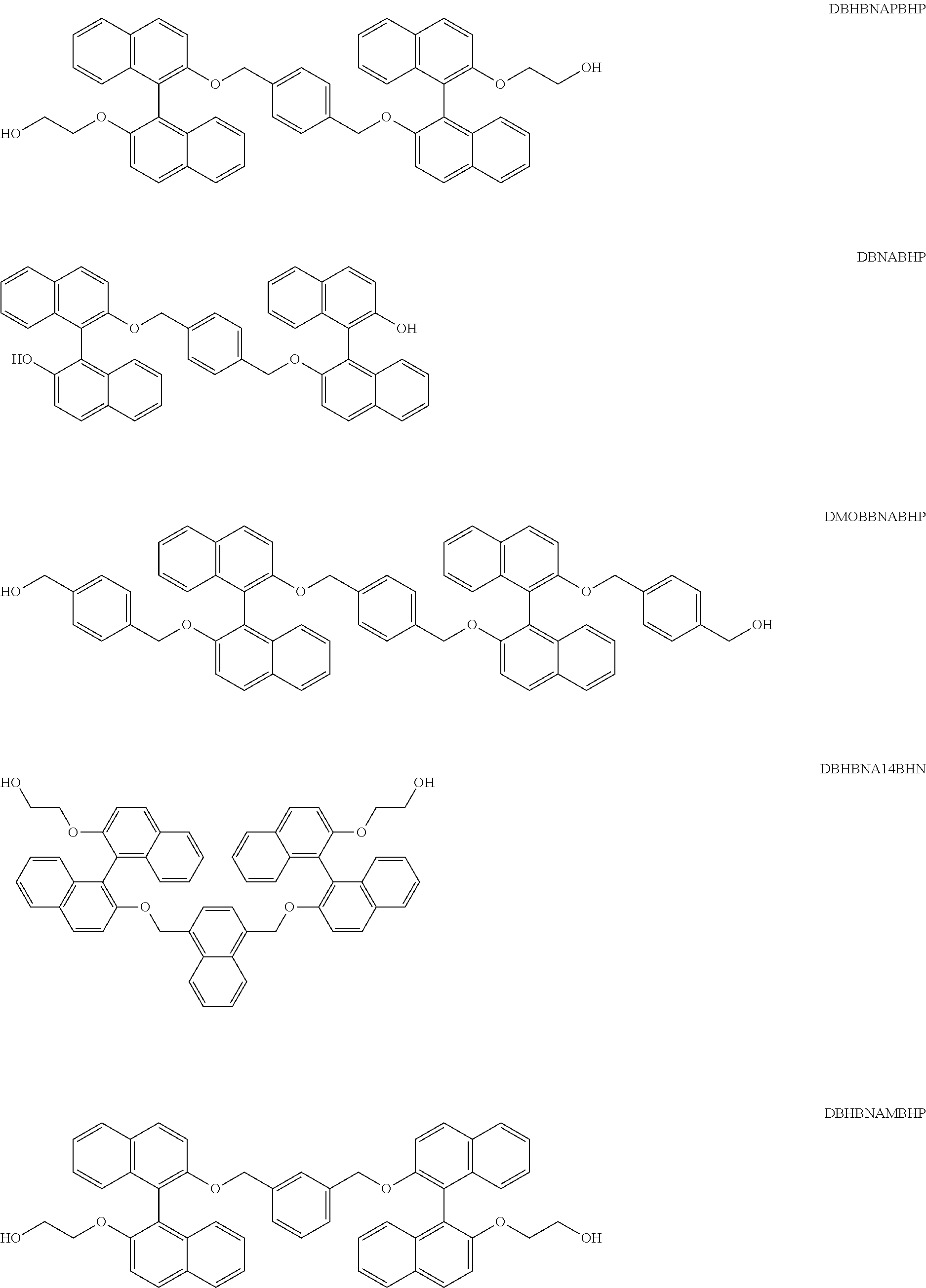 [SMILES: OCCOc1ccc2ccccc2c1-c1c(OCc2ccc(COc3ccc4ccccc4c3-c3c(OCCO)ccc4ccccc34)cc2)ccc2ccccc12 ; Oc1ccc2ccccc2c1-c1c(OCc2ccc(COc3ccc4ccccc4c3-c3c(O)ccc4ccccc34)cc2)ccc2ccccc12 ; OCc1ccc(COc2ccc3ccccc3c2-c2c(OCc3ccc(COc4ccc5ccccc5c4-c4c(OCc5ccc(CO)cc5)ccc5ccccc45)cc3)ccc3ccccc23)cc1 ; OCCOc1ccc2ccccc2c1-c1c(OCc2ccc(COc3ccc4ccccc4c3-c3c(OCCO)ccc4ccccc34)c3ccccc23)ccc2ccccc12 ; OCCOc1ccc2ccccc2c1-c1c(OCc2cccc(COc3ccc4ccccc4c3-c3c(OCCO)ccc4ccccc34)c2)ccc2ccccc12]

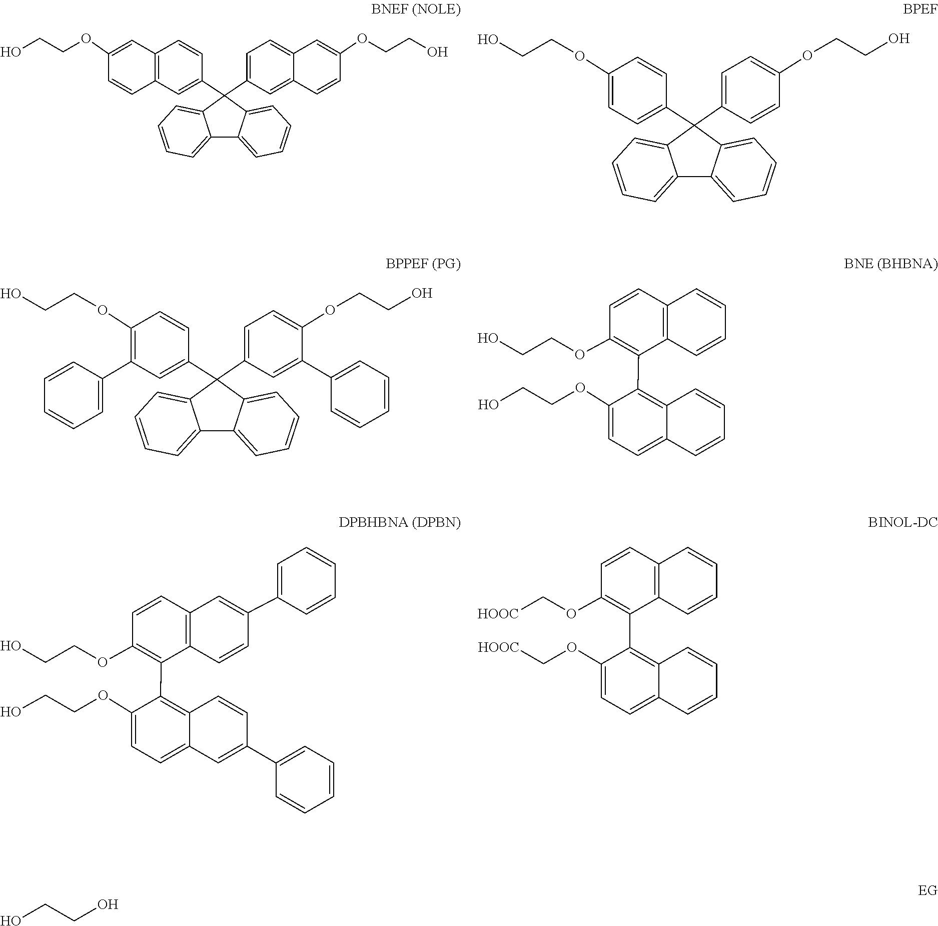

1. (canceled)

2. A compound of the formula (I) 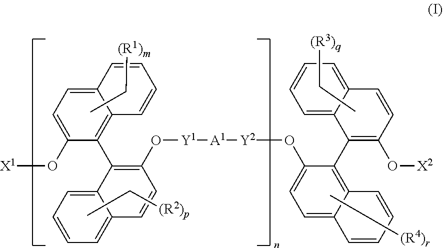
where
X¹ and X² are independently selected from hydrogen, -Alk¹-OH, —CH₂-A²-CH₂—OH, -Alk²-C(O)OR^{x}, —CH₂-A²-C(O)OR^{x} and —C(O)-A²-C(O)OR^{x}, where R^{x} is selected from the group consisting of hydrogen, phenyl, benzyl and C₁-C₄-alkyl;
Y¹ and Y² are independently selected from —CH₂—, —CHAr^{Y}- and CH(CH₂Ar^{Y})—;
A¹ is selected from the group consisting of a single bond, —CH₂—, —CHAr^{A}—, —CH(CH₂Ar^{A})-, —C(CH₂Ar^{A})₂-, a moiety of the formula (A), mono- or polycyclic arylene having from 6 to 26 carbon atoms as ring members and mono- or polycyclic hetarylene having a total of 5 to 26 atoms, which are ring members, where 1, 2, 3 or 4 of these ring member atoms of hetarylene are selected from nitrogen, sulfur and oxygen, while the remainder of these ring member atoms of hetarylene are carbon atoms, where mono- or polycyclic arylene and mono- or polycyclic hetarylene are unsubstituted or carry 1, 2, 3 or 4 radicals R^{Ar}, 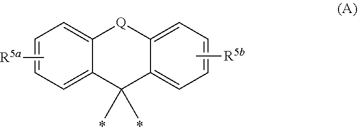
where
Q represents a single bond, O, C═O, CH₂, S or SO₂; and
R^{5a}, R^{5b}, independently of each other are selected from the group consisting of hydrogen, fluorine, CN, R, OR, CHₖR′₃₋ₖ, NR₂, C(O)R and C(O)NH₂, where k is 0, 1 or 2; and
* represents the connection point to Y¹ or Y²;
alternatively, the moiety —Y¹-A¹-Y²— in formula I may be —CH₂— or —CHAr^{Y}-,
n is 1, 2 or 3;
R¹, R², R³ and R⁴ are independently selected from the group consisting of hydrogen, halogen, C₂-C₃-alkynyl, CN, R, OR, CHₛR′₃₋ₛ, NR₂, C(O)R and CH═CHR″, it being possible that R¹, R², R³ or R⁴ is identical or different if more than 1 is present, where s on each occurrence is 0, 1 or 2;
m, p, q and r are independently 0, 1 or 2;
A² is selected from the group consisting of phenylene, naphthylene and biphenylylene;
Alk¹ is C₂-C₄-alkandiyl;
Alk² is C₁-C₄-alkandiyl;
Ar^{Y} and Ar^{A} are selected from the group consisting of mono- or polycyclic aryl having from 6 to 26 carbon atoms as ring atoms and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetaryl are carbon atoms, where Ar^{Y} and Ar^{A} are unsubstituted or substituted by 1, 2 or 3 radicals R^{Ar};
R^{Ar} is selected from the group consisting of R, OR, CHₜR′₃₋ₜ, NR₂ and CH═CHR″, where R^{Ar} may identical or different if more than one is present on the same (het)aryl or (het)arylene group, where t on each occurrence is 0, 1 or 2;
R is selected from the group consisting of C₁-C₄-alkyl, phenyl, naphthyl, phenanthrenyl and triphenylenyl, where phenyl, naphthyl, phenanthrenyl and triphenylenyl are unsubstituted or substituted by 1, 2, 3 or 4 identical or different radicals R′″;
R′ is selected from the group consisting of phenyl, naphthyl, phenanthrenyl and triphenylenyl, where phenyl, naphthyl, phenanthrenyl and triphenylenyl are unsubstituted or substituted by 1, 2, 3 or 4 identical or different radicals R′″;
R″ is selected from hydrogen, methyl, phenyl and naphthyl, where phenyl and naphthyl are unsubstituted or substituted by 1, 2, 3 or 4 identical or different radicals R′″;
R′″ is selected from the group consisting of phenyl, OCH₃, CH₃, N(CH₃)₂ and C(O)CH₃;
except for compounds of formula (I), where X¹ and X² are both hydrogen or —CH₂CH₂—OH, Y¹ and Y² are both CH₂, and A¹ is a single bond or CH₂; and except for compounds of formula (I), where n is 1, X¹ and X² are both hydrogen, Y¹ and Y² are both CH₂, m, p, q and r are all 0, and A¹ is 1,2-phenylene, 1,3-phenylene, 1,6-pyrenylene, 4,4′-biphenylylene, 2,6-pyridinylene, 4,4″-meta-terphenylylene, 2,5-[1,3,4]-thiadiazolylene, 2,5-[1,3,4]-oxadiazolylene, 2,5-thienediyl-bis(4,1-phenylenemethylene), 9,9-diethyl-2,7-9H-fluorenylene, 10-methyl-3,7-phenothiazinylene or 10-ethyl-3,7-phenothiazinylene.

3. The compound of claim 2, where X¹ and X² are selected from -Alk¹-OH and —CH₂-A²-CH₂—OH.

4. The compound of claim 2, where X¹ and X² are both hydrogen.

5. The compound of claim 2, where X¹ and X² are selected from -Alk²-C(O)OR^{x} and —CH₂-A²-C(O)OR^{x}.

6. (canceled)

7. (canceled)

8. The compound of claim 2, where Y¹ and Y² are both —CH₂—.

9. The compound of claim 2, where A¹ is selected from the group consisting of a moiety of the formula (A), mono- or polycyclic arylene and mono- or polycyclic hetarylene, where mono- or polycyclic arylene and mono- or polycyclic hetarylene are unsubstituted or carry 1, 2, 3 or 4 radicals R^{Ar}.

10. The compound of claim 2, where A¹ is selected from the group consisting of phenylene, naphthylene, 1,2-dihydroacenaphthylene, biphenylylene, 9H-fluorenylene, 11H-benzo[a]fluorenylene, 11H-benzo[b]fluorenylene, 7H-benzo[c]fluorenylene, anthracylene, phenanthrylene, benzo[c]phenanthrylene, pyrenylene, chrysenylene, picenylene, triphenylenylene, furanylene, benzo[b]furanylene, dibenzo[b,d]furanylene, naphtho[1,2-b]furanylene, naphtho[2,3-b]furanylene, naphtho[2,1-b]furanylene, benzo[b]naphtho[1,2-d]furanylene, benzo[b]naphtho[2,3-d]furanylene, benzo[b]naphtho[2,1-d]furanylene, benzo[1,2-b:4,3-b′]difuranylene, benzo[1,2-b:6,5-b′]difuranylene, benzo[1,2-b:5,4-b′ ]difuranylene, benzo[1,2-b:4,5-b′]difuranylene, 9H-xanthylene, tribenzo[b,d,f]oxepinylene, dibenzo[1,4]dioxinylene, 2H-naphtho[1,8-d,e][1,3]dioxinylene, phenoxathiinylene, dinaphtho[2,3-b:2′,3′-d]furanylene, oxanthrenylene, benzo[a]oxanthrenylene, benzo[b]oxanthrenylene, thienylene, benzo[b]thienylene, dibenzo[b,d]thienylene, naphtho[1,2-b]thienylene, naphtho[2,3-b]thienylene, naphtho[2,1-b]thienylene, benzo[b]naphtho[1,2-d]thienylene, benzo[b]naphtho[2,3-d]thienylene, benzo[b]naphtho[2,1-d]thienylene, benzo[1,2-b:4,3-b′ ]dithienylene, benzo[1,2-b:6,5-b′]dithienylene, benzo[1,2-b:5,4-b′]dithienylene, benzo[1,2-b:4,5-b′]dithienylene, 9H-thioxanthylene, 6H-dibenzo[b,d]thiopyranylene, 1,4-benzodithiinylene, naphtho[1,2-b][1,4]dithiinylene, naphtho[2,3-b][1,4]dithiinylene, 9H10-thia-anthracylene, thianthrenylene, benzo[a]thianthrenylene, benzo[b]thianthrenylene, dibenzo[a,c]thianthrenylene, dibenzo[a,h]thianthrenylene, dibenzo[a,i]thianthrenylene, dibenzo[a,j]thianthrenylene, dibenzo[b,i]thianthrenylene, 2H-naphtho[1,8-b,c]thienylene, dibenzo[b,d]thiepinylene, dibenzo[b,f]thiepinylene, 5H-phenanthro[4,5-b,c,d]thiopyranylene, tribenzo[b,d,f]thiepinylene, 2,5-dihydronaphtho[1,8-b,c:4,5-b′, c′]dithienylene, 2,6-dihydronaphtho[1,8-b,c:5,4-b′,c′]dithienylene, tribenzo[a,c,i]thianthrenylene, benzo[b]naphtho[1,8-e,f][1,4]dithiepinylene, dinaphtho[2,3-b:2′,3′-d]thienylene, 5H-phenanthro[1,10-b,c]thienylene, 7H-phenanthro[1,10-c,b]thienylene, dibenzo[d,d′]benzo[1,2-b:4,5-b′]dithienylene and dibenzo[d,d′]benzo[1,2-b:5,4-b′]dithienylene.

11. (canceled)

12. The compound of claim 2, where A¹ is selected from a single bond, —CH₂—, —CHAr^{A}—, —CH(CH₂Ar^{A})- and —C(CH₂Ar^{A})₂-, and in particular is —C(CH₂Ar^{A})₂-.

13. The compound of claim 2, where —Y¹-A¹-Y²— in formula (I) is —CH₂— or —CHAr^{Y}-, in particular is —CH₂—.

14. (canceled)

15. The compound of claim 2, where m, p, q and r are all 0.

16. The compound of claim 2, where formula (I) is represented by formula (Ia), where X is as defined as X¹ and X² in claim 2:

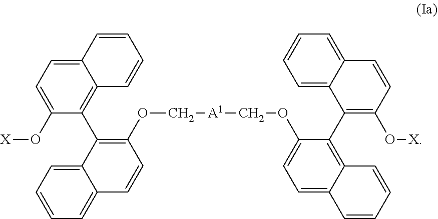

17. (canceled)

18. The compound of claim 13, where in formula (I) the moiety —Y¹-A¹-Y²— is —CH₂—, X¹ and X² are both 2-hydroxyethyl, m, p, q and r are all 0 and n is 1.

19. A thermoplastic resin comprising a structural unit represented by formulae (II) below 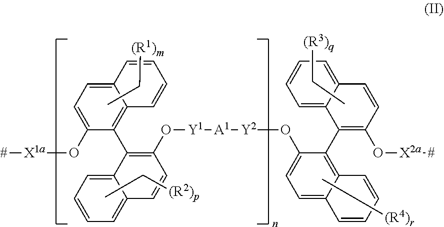
where
#represents a connection point to a neighboring structural unit;
and where X¹a and X^{2a}, respectively, is derived from X¹ or X² in formula (I), if X¹ or X² is hydrogen, by replacing hydrogen with a single bond, or, if X¹ or X² is not hydrogen, by replacing the —OH or —OR^{x} group of X¹ or X² with an oxo (—O—) unit, and where X¹, X², Y¹, Y², A¹, R¹, R², R³, R⁴, n, m, p, q and r are as defined in claim 2.

20. The thermoplastic resin of claim 19, which is of the formula (IIa), where X^{a} is as defined as X^{1a} and X^{2a} in claim 19:

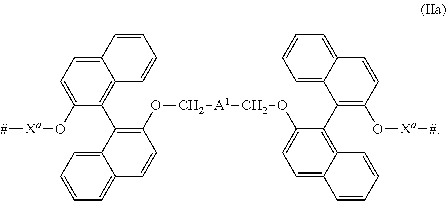

21. The thermoplastic resin of claim 19 where the structural unit of the formula (II), where X^{1a} and X^{2a} is selected from a single bond, -Alk¹-O— and —CH₂-A²-CH₂—O—, is connected to one of the structures represented by formulae (III-1) to (III-5) below, 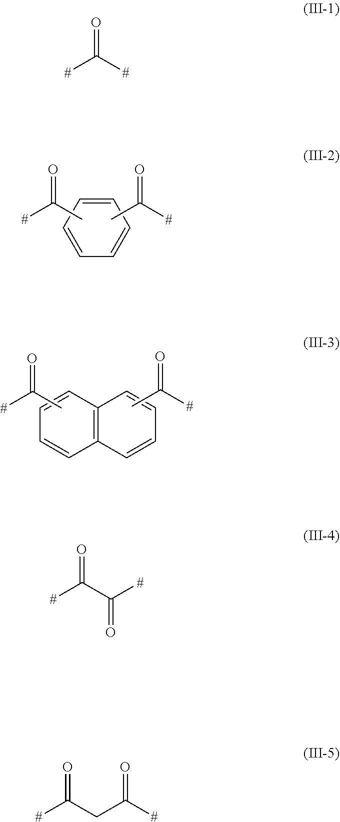
where
#represents a connection point to a neighboring structural unit.

22. The thermoplastic resin of claim 19, which is selected from copolycarbonate resins, copolyestercarbonate resins and copolyester resins, where the thermoplastic resin in addition to structural units represented by formula (II) comprises a structural unit of the formula (V),
#-O—R^{z}-A³-R^{z}—O-#- (V)
where
#represents a connection point to a neighboring structural unit;
A³ is a polycyclic radical bearing at least 2 benzene rings, wherein the benzene rings may be connected by W and/or directly fused to each other and/or fused by a nonbenzene carbocycle and/or fused by two non-benzene carbocycles that are linked via a linker L, where A³ is unsubstituted or substituted by 1, 2 or 3 radicals R^{aa}, which are selected from the group consisting of halogen, C₁-C₆-alkyl, C₅-C₆-cycloalkyl, phenyl, naphthyl, 1,2-dihydroacenaphthylenyl, phenanthrenyl, pyrenyl, triphenylenyl, benzo[b]furanyl, dibenzo[b,d]furanyl, benzo[b]thienyl, dibenzo[b,d]thienyl and thianthrenyl;
W is selected from the group consisting of a single bond, O, C═O, S, S(O), SO₂, CH₂, CH-Ar, CAr₂, CH(CH₃), C(CH₃)₂ and a radical of the formula (A′) 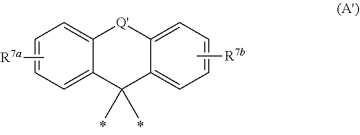
where
Q′ represents a single bond, O, C═O or CH₂;
R^{7a}, R^{7b}, independently of each other are selected from the group consisting of hydrogen, fluorine, CN, R, OR, CHᵥR′₃₋ᵥ, NR₂, C(O)R and C(O)NH₂, where R and R′ are as defined in claim 1 and v is 0, 1 or 2; and
* represents the connection point to a benzene ring;
L is selected from a single bond, C₁-C₄-alkylene, C₄-C₇-cycloalkylene, C₄-C₇-cycloalkylenedimethylene, phenylenedimethylene, where L is unsubstituted or substituted by 1 or 2 radicals R^{L}, which are selected from the group consisting of C₁-C₄-alkyl, halogen, C₁-C₄-haloalkyl, C₄-C₇-cycloalkyl and phenyl,
Ar is selected from the group consisting of mono- or polycyclic aryl having from 6 to 26 carbon atoms as ring atoms and mono- or polycyclic hetaryl having a total of 5 to 26 atoms, which are ring members, where 1, 2, 3 or 4 of these ring member atoms of hetaryl are selected from nitrogen, sulphur and oxygen, while the remainder of these ring member atoms of hetaryl are carbon atoms, where Ar is unsubstituted or substituted by 1, 2 or 3 radicals R^{ab}, which are selected from the group consisting of halogen, phenyl and C₁-C₄-alkyl;
R^{z} is a single bond, Alk³, O-Alk⁴-, O-Alk⁴-[O-Alk⁴-]W_{w} or O-Alk⁵-C(O)— where O is bound to A³, and where
w is an integer from 1 to 10;
Alk³ is C₁-C₄-alkandiyl;
Alk⁴ is C₂-C₄-alkandiyl; and
Alk⁵ is C₁-C₄-alkandiyl.

23. The thermoplastic resin of claim 22, where the structural unit of the formula V is represented by one of the following formulae V-1 to V-8: 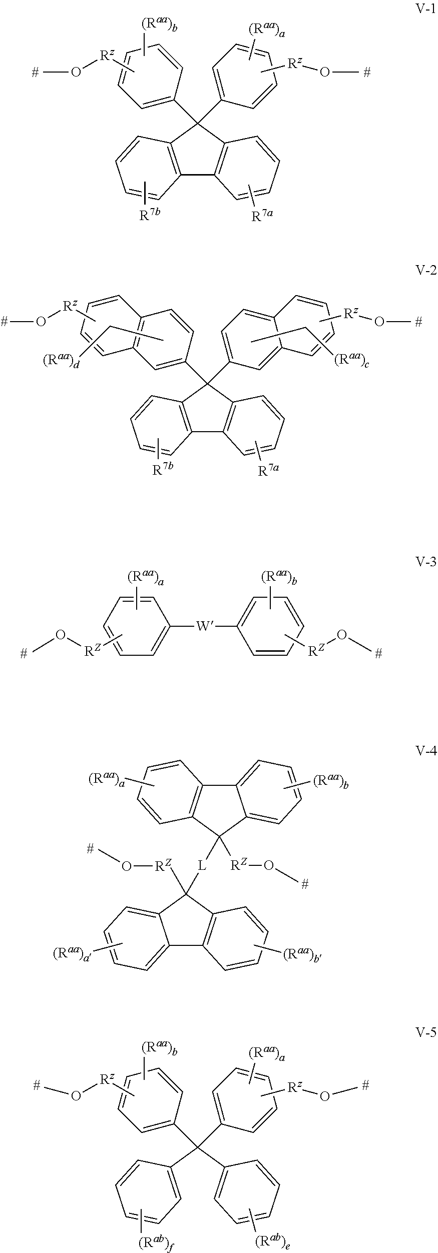 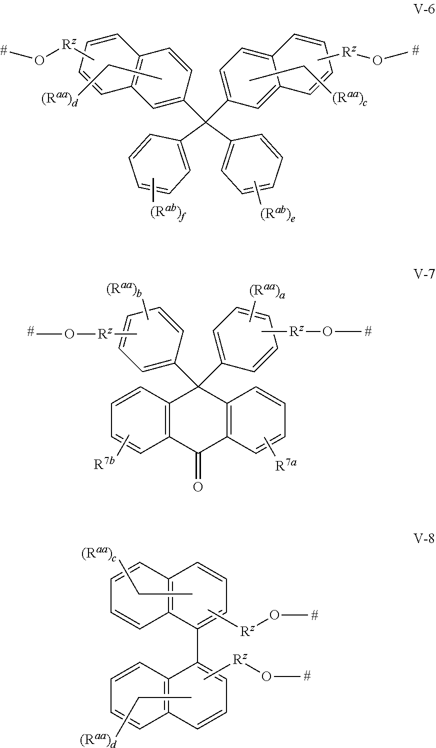
where
a and b are 0, 1, 2 or 3, in particular 0 or 1;
a′ and b′ are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1;
W′ is S, S(O), SO₂, O, single bond, CH₂, CH(CH₃), C(CH₃)₂, in particular S, S(O), SO₂ or C(CH₃)₂,
and where R^{z}, R^{aa}, R^{ab}, R^{7a}, R^{7b} and L are as defined for formula (V).

24. The thermoplastic resin of claim 22, where the molar ratio of the structural units of the formula (II) is from 1 to 99 mol-%, preferably 30 to 98 mol-%, based on the total molar amount of structural units of the formulae (II) and (V) and where the molar ratio of the structural units of the formula (V) is from 1 to 99 mol-%, preferably 2 to 70 mol-%, based on the total molar amount of structural units of the formulae (II) and (V).

25. The thermoplastic resin of claim 19, which has a refractive index of 1.630 or higher.

26. The thermoplastic resin of claim 19, which has an Abbe number of 24 or lower.

27. (canceled)

28. (canceled)

29. The thermoplastic resin of claim 19, which comprises 7% by weight or less of low molecular weight compounds having molecular weights of below 1000, based on the total weight of the thermoplastic resin.

30. The thermoplastic resin of claim 19, which comprises 1% by weight or more of low molecular weight compounds having molecular weights of below 1000, based on the total weight of the thermoplastic resin.

31. The thermoplastic resin of claim 19, where the thermoplastic resin is a polycarbonate, polyestercarbonate or polyester.

32. An optical device made of a thermoplastic resin as defined in claim 19.

33. The thermoplastic resin of claim 20, where the structural unit of the formula (II), where X^{1a} and X^{2a} is selected from a single bond, -Alk¹-O— and —CH₂-A²-CH₂—O—, is connected to one of the structures represented by formulae (III-1) to (III-5) below, 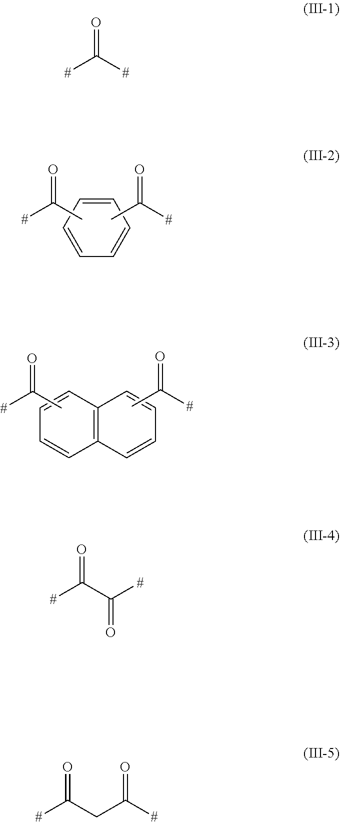
where
#represents a connection point to a neighboring structural unit.

34. The thermoplastic resin of claim 20, which is selected from copolycarbonate resins, copolyestercarbonate resins and copolyester resins, where the thermoplastic resin in addition to structural units represented by formula (II) comprises a structural unit of the formula (V),
#-O—R^{z}-A³-R^{z}—O-#- (V)
where
#represents a connection point to a neighboring structural unit;
A³ is a polycyclic radical bearing at least 2 benzene rings, wherein the benzene rings may be connected by W and/or directly fused to each other and/or fused by a nonbenzene carbocycle and/or fused by two non-benzene carbocycles that are linked via a linker L, where A³ is unsubstituted or substituted by 1, 2 or 3 radicals R^{aa}, which are selected from the group consisting of halogen, C₁-C₆-alkyl, C₅-C₆-cycloalkyl, phenyl, naphthyl, 1,2-dihydroacenaphthylenyl, phenanthrenyl, pyrenyl, triphenylenyl, benzo[b]furanyl, dibenzo[b,d]furanyl, benzo[b]thienyl, dibenzo[b,d]thienyl and thianthrenyl;
W is selected from the group consisting of a single bond, O, C═O, S, S(O), SO₂, CH₂, CH-Ar, CAr₂, CH(CH₃), C(CH₃)₂ and a radical of the formula (A′) 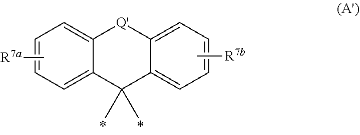
where
Q′ represents a single bond, O, C═O or CH₂;
R^{7a}, R^{7b}, independently of each other are selected from the group consisting of hydrogen, fluorine, CN, R, OR, CHᵥR′₃₋ᵥ, NR₂, C(O)R and C(O)NH₂, where R and R′ are as defined in claim 1 and v is 0, 1 or 2; and
* represents the connection point to a benzene ring;
L is selected from a single bond, C₁-C₄-alkylene, C₄-C₇-cycloalkylene, C₄-C₇-cycloalkylenedimethylene, phenylenedimethylene, where L is unsubstituted or substituted by 1 or 2 radicals R^{L}, which are selected from the group consisting of C₁-C₄-alkyl, halogen, C₁-C₄-haloalkyl, C₄-C₇-cycloalkyl and phenyl,
Ar is selected from the group consisting of mono- or polycyclic aryl having from 6 to 26 carbon atoms as ring atoms and mono- or polycyclic hetaryl having a total of 5 to 26 atoms, which are ring members, where 1, 2, 3 or 4 of these ring member atoms of hetaryl are selected from nitrogen, sulphur and oxygen, while the remainder of these ring member atoms of hetaryl are carbon atoms, where Ar is unsubstituted or substituted by 1, 2 or 3 radicals R^{ab}, which are selected from the group consisting of halogen, phenyl and C₁-C₄-alkyl;
R^{z} is a single bond, Alk³, O-Alk⁴-, O-Alk⁴-[O-Alk⁴-]_{w}- or O-Alk⁵-C(O)— where O is bound to A³, and where
w is an integer from 1 to 10;
Alk³ is C₁-C₄-alkandiyl;
Alk⁴ is C₂-C₄-alkandiyl; and
Alk⁵ is C₁-C₄-alkandiyl.
